(19) United States
(12) Patent Application Publication
(10) Pub. No.: US 2025/0316336 A1
(43) Pub. Date: Oct. 9, 2025

(54) SMALL CELL LUNG CANCER SUBTYPING USING PLASMA CELL-FREE NUCLEOSOMES

(71) Applicants: YISSUM RESEARCH DEVELOPMENT COMPANY OF THE HEBREW UNIVERSITY OF JERUSALEM LTD., Jerusalem (IL); UNITED STATES DEPARTMENT OF HEALTH AND HUMAN SERVICES, Bethesda, MD (US)

(72) Inventors: Ronen SADEH, Tel Aviv (IL); Nir FRIEDMAN, Mevaseret Zion (IL); Anish THOMAS, Bethesda, MD (US); Gavriel FIALKOFF, Jerusalem (IL); Sharkia ISRAA, Jatt (IL); Nobuyuki TAKAHASHI, Kashiwa-shi (JP)

(73) Assignees: YISSUM RESEARCH DEVELOPMENT COMPANY OF THE HEBREW UNIVERSITY OF JERUSALEM LTD., Jerusalem (IL); UNITED STATES DEPARTMENT OF HEALTH AND HUMAN SERVICES, Bethesda, MD (US)

(21) Appl. No.: 18/865,855

(22) PCT Filed: May 17, 2023
(86) PCT No.: PCT/IL2023/050509

§ 371 Date: Nov. 14, 2024

Related U.S. Application Data
(60) Provisional application No. 63/342,763, filed on May 17, 2022.

(51) Int. Cl. G16B 30/00; C12Q 1/6874; C12Q 1/6886; G16B 40/20

(52) U.S. Cl. CPC G16B 30/00; C12Q 1/6874; C12Q 1/6886; G16B 40/20

(57) ABSTRACT

Methods of determining disease load or type in a subject suffering from a disease associated with cell death of a specific tissue or cell type are provided. Methods of determining a cell free DNA chromatin immunoprecipitation and sequencing (cfChIP-Seq) marker and methods of classifying a subject suffering from a disease are also provided.

### CROSS REFERENCE TO RELATED APPLICATIONS

This application claims the benefit of priority of U.S. Provisional Patent Application No. 63/342,763 filed on May 17, 2022, the contents of which are all incorporated herein by reference in their entirety.

### FIELD OF INVENTION

The present invention is in the field of cancer and liver disease diagnostics.

### BACKGROUND OF THE INVENTION

Small cell lung cancer (SCLC) is a neuroendocrine lung cancer that is highly aggressive with dismal prognosis, accounting for approximately 15% of all lung cancers. SCLC sheds the largest amount of cfDNA of all solid tumors. Prior studies have identified cfDNA mutations in more than 80% of SCLC patients, but recurrent targetable mutations in known oncogenes, such as those seen in the kinases that comprise targetable drivers in lung adenocarcinoma, are rare in SCLC. Recurrent mutations also do not demonstrate consistent co-occurrence or mutual exclusivity, and thus do not define SCLC subtypes.

SCLCs exhibit high expression of neuronal and neuroendocrine transcription factors and MYC paralogs that drive a broad range of genes related to cell proliferation and growth signaling. Importantly, SCLC subtypes driven by distinct transcription factors have unique therapeutic vulnerabilities. However, identification of SCLC transcriptomic subtypes and their application in the context of subtype-specific therapies has proven challenging due to limited access to tumor specimens. The majority of SCLC patients do not undergo surgical resection as their disease is detected after it has spread beyond the primary site. Moreover, patients with relapsed disease generally deteriorate quickly, and recurrence suspected on imaging is typically followed by immediate treatment without biopsies. Highlighting this challenge, SCLC is represented in none of the large sequencing initiatives like The Cancer Genome Atlas and Pan-cancer Analysis of Whole Genomes.

Autoimmune hepatitis (AIH) is a rare chronic self-perpetuating inflammatory liver disease, characterized by immune-mediated damage to hepatocytes. The clinical presentation of AIH is heterogeneous and includes elevated serum transaminases and seropositivity of autoantibodies and immunoglobulin G, yet the final diagnosis requires histological evidence of hepatic inflammation and interface hepatitis with increased plasma cell which entails liver biopsy. Thus, like SCLC it requires an invasive diagnostic step in order to determine proper treatment.

Several lines of evidence suggest that hepatocyte damage in AIH is mediated by CD4+ T-cells, particularly the Th17 cells3, though the underlying mechanisms are not fully understood. Immunosuppression and liver transplantation in severe cases of liver failure or cirrhosis, are the sole therapeutic alternatives. Normalization of transaminase levels along with IgG levels and negative auto-antibodies define biochemical remission of the disease. Biochemical remission is usually a sufficient indication of successful response to treatment but does not always correlate with histological remission. Thus, in most cases liver biopsy is needed to confirm histological remission to allow stopping medications.

Identifying tumor-specific alterations in cell free DNA (cfDNA) presents a powerful opportunity to reduce cancer morbidity and mortality. Most of the current clinical applications of cfDNA are centered around interrogating the mutational landscape, and as such are of limited utility in defining transcriptomic subtypes. Recently, chromatin immunoprecipitation and sequencing of cell-free nucleosomes from human plasma (cfChIP-seq) was used to infer the transcriptional programs of the cells of origin. Specifically, tri-methylation of histone 3 lysine 4 (H3K4me3) is a well characterized histone modification, marking transcription start sites (TSS) of genes that are poised or actively transcribed, and is predictive of gene expression.

The translational potential of the newly described SCLC transcriptional phenotypes and their associated vulnerabilities is limited by access to tumor biopsies. Similarly, final diagnosis of AIH and monitoring its response to treatment cannot be determined without liver biopsies. This is true in many other cancers as well. where biopsies may not be available or are difficult to obtain. A new accurate non-invasive test for identifying transcriptional phenotypes in cancer and other diseases is therefore greatly needed.

### SUMMARY OF THE INVENTION

The present invention provides methods of determining disease load or type in a subject suffering from a disease associated with cell death of a specific tissue or cell type are provided. Methods of determining a cell free DNA chromatin immunoprecipitation and sequencing (cfChIP-Seq) marker and methods of classifying a subject suffering from a disease are also provided.

According to a first aspect, there is provided a method of determining disease load in a subject suffering from a disease associated with cell death of a specific tissue or cell type, the method comprising:
a. receiving chromatin immunoprecipitation-sequencing (ChIP-Seq) reads from a plurality of genomic locations from cell free DNA (cfDNA) from blood samples from
   i. a first population of control subjects;
   ii. a second population of subjects suffering from the disease; and
   iii. the subject; and
b. assigning a disease load score to the subject based on the similarity of the subject's reads to reads from the second population and dissimilarity to reads from the first population, wherein the score is proportional to the disease load in the subject;
thereby determining disease load in a subject.

According to some embodiments, the ChIP-Seq was performed with an antibody to a DNA associated protein that marks active transcription.

According to some embodiments, the DNA associated protein that marks active transcription is selected from: histone H3 lysine 4 trimethylation (H3K4me3), histone H3 lysine 27 acetylation (H3K27Ac), histone H3 lysine 36 trimethylation (H3K36me3), histone H3 lysine 4 monomethylation (H3K4me) and histone H3 lysine 4 dimethylation (H3K4me2).

According to some embodiments, the DNA associated protein that marks active transcription is H3K4me3.

According to some embodiments, the similarity is determined by a linear regression analysis.

According to some embodiments, the similarity is determined by a trained machine learning algorithm, wherein the machine learning algorithm is trained on ChIP-Seq reads from cfDNA from blood samples from the first population and the second population and labels identifying the ChIP-Seq reads as being from a subject of the first population or a subject of the second population.

According to some embodiments, the control subjects are healthy subjects.

According to some embodiments, the second population is a subset of the second population, wherein the subset comprises the top 10% of the second population with the most differentially expressed genes, based on ChIP-Seq reads, as compared to the first population.

According to some embodiments, the disease is a specific type of cancer and the disease load score is a cancer load score.

According to some embodiments, the control subjects are subjects that suffer from a cancer of a different type than the specific type of cancer.

According to some embodiments, the cancer is lung cancer.

According to some embodiments, the lung cancer is small cell lung cancer (SCLC) and wherein the score is specific to SCLC and not other cancers.

According to some embodiments, a disease score beyond a predetermined threshold indicates the subject suffers from SCLC.

According to some embodiments, the disease is a specific liver disease and the disease load score is a liver disease load score.

According to some embodiments, the control subject are subjects that suffer from a liver disease other than the specific liver disease.

According to some embodiments, the specific liver disease is autoimmune hepatitis (AIH), and wherein the liver disease load score is specific to AIH and not other liver diseases.

According to some embodiments, a disease score beyond a predetermined threshold indicates the subject suffers from AIH.

According to some embodiments, the receiving ChIP-Seq reads comprises:
a. receiving a blood sample from the subject, a subject of the first population, a subject from the second population or any combination thereof;
b. contacting the sample with at least one reagent that binds to a DNA-associated protein indicative of active transcription;
c. isolating the reagent and any thereto bound proteins and cfDNA; and
d. sequencing the cfDNA.

According to another aspect, there is provided a method of determining a ChIP-Seq marker that distinguishes cfDNA from a first disease from cfDNA from a second disease, the method comprising:
a. determining disease load in a plurality of subjects suffering from the first disease by a method of the invention wherein the control subjects are healthy subjects or subjects suffering from a different disease;
b. selecting a subset of the plurality of subjects with a disease load above a predetermined threshold; and
c. comparing ChIP-Seq reads from cfDNA from blood from the subset with ChIP-Seq reads from cfDNA from blood of a third population of subjects suffering from the second disease and selecting at least one genomic region with a differential signal between the subset and the third population;
thereby determining a ChIP-Seq marker.

According to some embodiments, the comparing is comparing ChIP-Seq reads from genomic regions with a differential signal between the first population and the second population.

According to some embodiments, the method is a method of determining markers for a cancer subtype, wherein the first disease and second disease are cancer of the same type, the same tissue or cell type and the first disease is a first subtype of the cancer and the second disease is a second subtype of the cancer.

According to some embodiments, the cancer is SCLC and the method is a method of determining a marker for a SCLC subtype.

According to some embodiments, the genomic regions are from within a gene body or regulatory element of a gene.

According to some embodiments, the regulatory element is a promoter.

According to some embodiments, the gene is a transcription factor or transcriptional coregulator.

According to another aspect, there is provided a method of classifying a subject as suffering from a first disease, the method comprising:
a. determining a ChIP-Seq marker for the first disease by a method of the invention;
b. receiving ChIP-Seq reads from cfDNA from a blood sample from the subject; and
c. identifying reads of the determined ChIP-Seq marker in the received ChIP-Seq reads, wherein reads above a predetermining threshold indicate the subject suffers from the first disease;

thereby classifying a subject as suffering from a first disease.

According to some embodiments, the method further comprises administering to the subject a therapeutic agent that treats the first disease.

According to another aspect, there is provided a method of assigning a subject suffering from SCLC to a SCLC subtype, the method comprising:
a. receiving ChIP-Seq reads from cfDNA from a blood sample from the subject; and
b. identifying reads from at least one informative genomic locus as being above or below a predetermined threshold, wherein the reads are from a genomic locus provided in any one of Tables 1-3 wherein the SCLC subtype is selected from: achaete-scute homolog 1 (ASCL1) subtype, neurogenic differentiation 1 (NEUROD1) subtype, POU domain class 2 transcription factor 3 (POU2F3) subtype, yes-associated protein 1 (YAP1) subtype and protein atonal homolog 1 (ATOH1) subtype;
thereby assigning a subject suffering from SCLC to a SCLC subtype.

According to some embodiments, the subtype is further selected from high neuroendocrine phenotype SCLC and non- or low-neuroendocrine phenotype SCLC.

According to some embodiments, the high neuroendocrine phenotype SCLC is a ASCL1 subtype, or a NEUROD1 subtype, and wherein the non- or low-neuroendocrine phenotype SCLC is a POU2F3 subtype, YAP1 subtype or an ATOH1 subject.

According to some embodiments, the subtype is selected from: ASCL1, NEUROD1, POU2F3 and ATOH1 subtypes.

According to some embodiments, the subtype is selected from: ASCL1, NEUROD1, and POU2F3 subtypes.

According to some embodiments, the reads are from a genomic locus provided in Table 1 and reads above a predetermined threshold indicate the SCLC is of the ASCL1 subtype.

According to some embodiments, the reads are from a genomic locus provided in Table 2 and reads above a predetermined threshold indicate the SCLC is of the NEUROD1 subtype.

According to some embodiments, the reads are from a genomic locus provided in Table 3 and reads above a predetermined threshold indicate the SCLC is of the POU2F3 subtype.

According to some embodiments, reads are from at least one genomic locus provided in each of Tables 1-3 and wherein reads from all genomic loci are below a predetermined threshold the SCLC is of the ATOH1 or YAP1 subtype.

According to some embodiments, the determined cancer subtype correlates with predicted subject survival time.

According to some embodiments, the method is a method of predicting survival time of the subject.

According to some embodiments, the method further comprises administering to the subject a therapeutic agent specific to the SCLC subtype.

According to some embodiments, the determined cancer subtype is high-neuroendocrine subtype and the therapeutic agent comprises chemotherapy.

According to some embodiments, the determined cancer subtype is non- or low-neuroendocrine subtype and the therapeutic treatment comprises immunotherapy.

According to some embodiments, the immunotherapy is immune checkpoint blockade, optionally wherein the immune checkpoint is PD-1/PD-L1.

According to another aspect, there is provided a method of diagnosing or prognosing AIH in a subject, the method comprising:
a. receiving ChIP-Seq reads from cfDNA from a blood sample from the subject; and
b. identifying reads from an informative genomic locus as being above a predetermined threshold, wherein the reads are from a genomic locus within a gene body or promoter of a gene selected from: BCL2L14, CXCL10, CXCL11, CXCL9, GBP1, GBP5, HAPLN3, HLA-DOB, IL32, KB-1615E4.2, MARVELD3, OAS2, TRIM31, UBD, UPP2;
thereby diagnosing or prognosing AIH in a subject.

According to some embodiments, the method is a method of detecting AIH in a subject.

According to some embodiments, the method further comprises administering an anti-AIH therapeutic agent to a subject diagnosed with AIH.

According to some embodiments, the method is a method of monitoring AIH in a subject being administered an anti-AIH therapeutic agent.

According to some embodiments, the method further comprises continuing to administer the anti-AIH therapeutic agent to a subject determined to have residual AIH.

According to some embodiments, the anti-AIH therapeutic agent is an immunosuppressant, optionally wherein the immunosuppressant is a steroid.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1G: SCLC plasma samples exhibit distinct cfChIP-seq signals that correlate with tumor burden and survival. (1A) Study outline. Plasma samples were collected from a cohort of healthy individuals and patients with SCLC at various time points during treatment. cfChIP-seq was performed on plasma samples and tumor biopsy samples—many of them time-matched with plasma draw—profiled using RNA-seq. Plasma cfChIP-seq-based gene expression profiles were benchmarked against tumor RNA-seq, and key SCLC transcription regulators examined. (1B) Genes with significantly high coverage in selected SCLC (n=15) samples (Methods) compared to healthy baseline. For each gene we compare mean normalized coverage in the SCLC samples (y-axis) to a healthy cohort reference (x-axis). 3642 genes with significantly higher coverage (q<0.001) in at least 3 SCLC samples are marked. Genes of particular interest: CHCA, DLL3, EGFR, FOXA2, INSM1 and NFIB are marked. (1C) Median SCLC-score in different cohorts as calculated by linear regression (Methods). Each dot is a sample, and boxplots summarize distribution of SCLC-scores in each group. Pre/post refers to the time point along treatment. ****: P<0.0001. (1D) Principal components 1 and 2 of SCLC (orange) and healthy (green) samples. Transparency indicates SCLC-score as in C. Principal components analysis was done using all genes (˜40,000). (1E) Correlation of SCLC-score with other plasma and imaging-based measures of tumor burden. Abbreviations: RECIST: Response Evaluation Criteria in Solid Tumors. (1F) Changes of SCLC-score and radiological tumor burden in a patient with SCLC over the treatment time-course (CL0191). Abbreviations: Tr. 1—durvalumab & olaparib; Tr. 2—Topotecan & M6620; RT—radiation; Tr. 3—investigational therapy; CR—complete response; SD—stable disease; PD—progressive disease. (1G) Kaplan Meier curve of overall survival in patients with SCLC-score above (red; median survival: 3 month) or below (blue; median survival: 8 month) median. p calculated by log-rank test.

FIGS. 2A-2G: cfChIP-seq recovers SCLC tissue and cellular origins. (2A) Heatmap showing patterns of the relative cfChIP-seq coverage of SCLC signature genes. The normalized coverage on the gene promoter was log-transformed (log 2 (1+coverage)) and adjusted to zero mean for each gene across the samples. (2B) Median and distribution of SCLC and healthy sample coverage on genes shown in A. ****: P<0.0001 (2C) Genome browser view of cfChIP-seq signal in canonical SCLC genes (DLL3, INSM1, CHGA, SYP) and GAPDH as control. Orange and green tracks represent SCLC and healthy samples respectively. (2C) Genome browser view of cfChIP-seq signal in canonical SCLC genes (DLL3, INSM1, CHGA, SYP) and GAPDH as control. Orange and green tracks represent SCLC and healthy samples respectively. (2D) Median and distribution of the cumulative cfChIP-seq coverage over the SCLC-signature genes. (2E) Cell and tissue of origin signatures in healthy and SCLC samples. x-axis values indicate absolute contribution of signature (normalized reads/kb corrected by estimated cfDNA concentration; methods) Neutrophils, monocytes and megakaryocytes are observed in both groups, while lung, brain and B-cells are observed only in SCLC samples. (2F) Single cell RNA-seq-derived lung cell-type signatures in healthy and SCLC samples. Values (x-axis) indicate the sum of normalized reads in the marker genes of every tissue. An increased signal is observed in many of these cell-types in SCLC samples only. (2G) Ratio for signal in cell-types shown in F of high-score SCLC samples (n=31) compared to Roadmap lung ChIP-seq. A significant elevation is observed in neuroendocrine and ciliated cells.

FIGS. 3A-3D: Plasma cfChIP-seq informs tumor gene expression. (3A) Concept figure illustrating the various sources and proportions of cells sampled in plasma cfChIP-seq and tumor RNA-seq. The data obtained from these two assays is derived from a different unknown mixture of multiple types of cells. (3B) Comparison of gene expression in two high SCLC-score samples. Top: TMM-normalized FPKM of tumor RNA-seq. Bottom: normalized gene counts of cfChIP-seq. Blue and red points indicate genes that had high cfChIP-seq gene counts in one sample compared to the other and low gene counts in healthy reference cfChIP. (3C) Gene level analysis of the correlation between tumor gene expression and plasma cfChIP-seq coverage across individuals with matched tumor and plasma samples (left: all SCLC samples. right: high SCLC-score samples). For each gene we computed the Pearson correlation of its tumor expression and the normalized cfChIP-seq coverage across the samples. Shown is a histogram of the correlations on genes with high dynamic ranges (Methods). In gray is the histogram of a random permutation of the relation between tumor expression and plasma cfChIP. (3D) Examples of correlation for several known SCLC oncogenes.

FIGS. 4A-4D: cfChIP-seq displays differential expression of SCLC transcription drivers. (4A) Correlation of NE-score computed based on plasma cfChIP-seq and tumor RNA-seq. Only plasma samples with matching tumors and high SCLC-score are presented. (4B) Heatmap showing relative expression levels of 5 canonical SCLC transcription drivers across the tumor RNA-seq samples. Values are presented in log 2 (1+TMM normalized-FPKM) and adjusted to zero mean for each sample across the 5 genes. Transcription drivers' expression patterns are generally mutually exclusive. (4C) Median and distribution of SCLC and healthy cfChIP-seq plasma sample coverage on the gene shown in B. ****p<0.0001. (4D) Correlation of plasma cfChIP-seq coverage and tumor RNA-seq of the genes shown in C. Only plasma samples with matching tumors and high SCLC-score are presented.

FIGS. 5A-5E: SCLC subtyping using cfChIP derived signatures. (5A) Heatmap showing cfChIP-seq coverage patterns of subtype specific genomic regions (rows) across samples of the different subtypes (columns). Color scale represents the normalized coverage on the genomic region after log-transformation (log 2 (1+coverage)) and adjusting to zero mean across the samples. Rows are divided into three different signatures: Top-ASCL1 specific regions, Middle-NEUROD1 specific regions, Bottom-POU2F3 specific regions. Columns divide all samples into five subtype groups (ASCL1, ASCL1-NEUROD1, NEUROD1, POU2F3 and YAP1) based on RNA levels in the matching tumors. Ranking within every group is determined by the SCLC-score. Bottom bars display the log 2 (1+TMM normalized-FPKM) RNA-seq levels of the four major SCLC transcription regulators in the matching tumor. (5B) Subtype-specific signatures strength (y-axis) increases linearly with SCLC-score (x-axis) in samples of corresponding subtypes, but are constant in samples of other subtypes. x-axis indicates aggregated reads in the genomic regions shown in 5A. Points are colored by the sample subtype as determined by RNA-seq from matching tumors (gray points represent plasma samples from healthy individuals). Dotted vertical lines represent a 0.05 SCLC-score cutoff of samples that can be classified using subtype specific signatures. (5C) Median and distribution of the signatures' score across all samples. The signature score in the y-axis indicates the cumulative reads in every signature (as in 5B) normalized by the SCLC-score of every sample. Samples with very low ctDNA contribution (to the left of the dotted line in 5B) are not presented. Dotted line represents the classifier cutoff values. A=ASCL1, A+N=ASCL1+NEUROD1, N-NEUROD1, P=POU2F3 and Y=YAP1. (5D) Comparison of signature scores (as in C) across samples. Dotted lines represent the classifier cutoff values. (5E) Schematic workflow of subtype classifier. All samples with SCLC-score above 0.05 are evaluated for the POU2F3 signature score. Samples above the cutoff are classified as POU2F3, and the remaining samples are evaluated for both ASCL1 and NEUROD1 signature score.

FIGS. 6A-6G: SCLC-score correlates with tumor load and predicts response to treatment. (6A) Distribution of the number of genes with significantly higher coverage in SCLC samples compared to healthy baseline (Methods). Dashed line represents the 90th percentile. (6B) Scree plot of PCA (FIG. 1D). (6C) Correlation of PC1 and SCLC-score (presented in FIG. 1C-D). High correlation indicates that the main variation in the data is driven by the SCLC contribution to the cfDNA. (6D) Correlation of SCLC-score and other plasma-based methods for tumor load estimations (cfDNA concentration, circulating tumor cells). (6E) Correlation of cfDNA concentration and ctDNA fraction. (6F) Dynamics of SCLC-score during treatments of topotecan and berzosertib (an ATR inhibitor) (ClinicalTrial.gov identifier NCT02487095). Gray lines indicate multiple timepoints of the same individual. (6G) Median SCLC-score in patients that did or did not respond to various investigational treatments: combination of topotecan and berzosertib (NCT02487095, NCT03896503); olaparib and durvalumab (NCT02484404); nanoparticle camptothecin (CRLX101) and olaparib (NCT 02769962); M7824 (PD-1 inhibitor and TGF-B trapping) and topotecan or temozolomide (NCT03554473). Each dot is a sample, and boxplots summarize distribution of values in each group. Abbreviations: ATR: ataxia telangiectasia and Rad3-related.

FIGS. 7A-7D: SCLC tissue and cell of origin. (7A) Distribution of signal for cell-type signatures (same as FIG. 2E). (7B) Correlation of cell-type signature (shown in FIG. 2E) and SCLC-score. Cell-types observed only in SCLC samples are positively correlated with SCLC-score, while cell-types observed in healthy samples are negatively correlated to SCLC-score. (7C) Left: number of marker genes used for every cell-type (Methods). Right: Distribution of signal for lung cell-type marker genes (same as FIG. 2F). **** and ***: P<0.0001 and <0.001, respectively. (7D) Heatmap showing patterns of signal in lung cell-type markers in the SCLC and healthy samples. Color represents log 2 (1+cumulative coverage in promoters of marker genes).

FIGS. 8A-8F: cfChIP-seq RNA correlation confounders. (8A) Comparison of signal ratio of the two individuals shown in FIG. 3A in tumor RNA and plasma cfChIP. Blue and Red points correspond to genes with higher cfChIP-seq counts in every sample. Lighter points represent genes with low RNA counts (combined TMM-FPKM<3). (8B) Gene level analysis of the correlation in tumor expression level and plasma cfChIP-seq levels (same as in FIG. 3B for all Refseq genes). (8C) Comparison of the effect of cfChIP-seq and RNA dynamic range (x-axis) on cfChIP-RNA correlation (y-axis) shown in FIG. 3B. Left panel for the observed correlation and right panel for a random permutation (gray histogram of FIG. 3B). Results indicate that genes with low ChIP dynamic range tend to have lower correlation. (8D) Same as C for SCLC/healthy ratio (x-axis). The ratio was computed using the mean gene counts of high SCLC samples and healthy samples. (8E) log 2 (TMM-FPKM) of tumor samples in liver specific genes. High expression is observed in samples where biopsy was obtained from the liver. (8F) Same as C for gene expression in the liver (x-axis).

FIGS. 9A-9D: cfChIP signal in SCLC lineage-driving genes. (9A) Genome browser view of cfChIP-seq signal in SCLC key transcriptional regulators. (9B) Correlation of plasma cfChIP-seq coverage in promoter and gene body and tumor RNA-seq of the genes POU2F3 and ATOH1. Noticeably, in these genes, the correlation to expression is higher in the gene body. (9C) Genome browser view of cfChIP-seq signal in panel D. Orange and green tracks represent SCLC and healthy samples respectively. While a signal in the gene promoter is observed in many samples, only in specific samples there is a significant coverage also in the gene body. (9D) Relation of the ratio of signal in markers of ciliated and neuroendocrine plasma ChIP samples (y-axis) and relative RNA expression of POU2F3 in the tumor biopsy (x-axis). Relative RNA POU2F3 was calculated by log 2 (POU2F3)/sum (log 2 (ASCL1, NEUROD1, YAP1, POU2F3)) where all RNA values are TMM-FPKM normalized.

FIGS. 10A-10C: SCLC subtyping using cfChIP derived signatures. (10A) Heatmap showing subtype signature (rows) across samples of the different subtypes (columns). Color scale represents the aggregate signal after log-transformation (log 2 (1+coverage) and zero mean adjustment on genomic regions shown in 5A. Columns divide all samples into five subtype groups (ASCL1, ASCL1-NEROD1, NEUROD1, POU2F3 and YAP1) based on RNA levels in the matching tumors. Ranking within every group is determined by the SCLC-score. (10B) ROC curve and AUROC score for cfChIP-based classifier of the signatures shown in A. ASCL1-NEUROD1 were considered positive for both ASCL1 and NEUROD1 signatures. (10C) Comparison of ASCL1 and NEUROD1 signatures scores across samples. Dotted lines represent the ASCL1 and NEUROD1 classifier cutoff values. Points are colored by the sample subtype as determined by RNA-seq from matching tumors.

FIGS. 11A-11D: cfChIP-seq displays elevated liver-derived cfDNA in AIH plasma samples. (11A) Study outline. Plasma samples were collected from a cohort of healthy individuals, patients with AIH and patients with other liver diseases. cfChIP-seq was performed on 1 ml of plasma to recover AIH cfDNA tissue-of-origin, infer transcriptional programs in dying cells and classify AIH samples. (11B) Detection of genes with significantly elevated coverage in a representative AIH plasma sample. For each gene, the mean normalized promoter coverage in the sample (y-axis) was compared to a reference healthy cohort (x-axis). Significance indicates whether the observed number of reads in the sample is significantly higher than expected based on the mean and variance in healthy samples (Methods). (11C) Distribution of the number of genes with significantly elevated promoter coverage in sample compared to healthy baseline (Methods). Samples were binned to three groups: samples with 0, less than 100 and more than 100 genes with significant coverage above healthy. Biochemical measurements (ALT, AST) were used to group AIH samples to active (>40 U/L) and remission (<40 U/L) states. (11D) Bottom: Hierarchical clustering of genes with significant coverage in at least 3 AIH samples (764 genes, rows) across cfChIP-seq and reference tissue and cell types ChIP-seq samples (columns). Color bar represents tissue category (liver, solid tissue or immune cells) as shown in the boxplot above. Color scale represents the sample's normalized reads per promoter region of the gene. Rows were hierarchically clustered and split Top: Median and distribution of cumulative signal of the same genes across reference tissues and cell types (right side of heatmap). The genes elevated in the AIH samples are enriched for the liver.

FIGS. 12A-12F: cfChIP-seq identifies hepatocyte as a major source of cfDNA in AIH plasma. (12A) Tissue composition median and distribution of healthy and AIH samples. y-axis values indicate relative contribution of tissue to the circulation. Neutrophils, monocytes and megakaryocytes are observed in both groups, while liver has a pronounced contribution only in the AIH samples. */**/***: t-test P<0.01/0.001/0.0001. Only significant comparisons are shown in Figure. (12B) Genome browser view of cfChIP-seq signal in hepatocytes marker genes (APOB, HPX, F12, HSD11B1) and ACTB as control. Green and red tracks represent healthy and AIH cfChIP-seq samples respectively. (12C) Heatmap showing patterns of cfChIP-seq samples (columns) coverage across liver cell-type marker genes (rows). Color represents normalized coverage at the gene promoter. Of all liver cell type markers, a noticeable signal in the AIH samples is observed only in the hepatocyte marker genes. (12D) Pearson correlation of cfChIP-seq liver fraction (y-axis) and time-matched blood ALT levels (x-axis). (12E) Principal components 1 and 2 of AIH (red and orange) and healthy (green) samples. Transparency indicates liver fraction as in A. Principal components analysis was computed over all Refseq genes (˜25,000). (12F) Pearson correlation of PC1 and liver fraction displayed in D. Liver fraction differences across samples explains the largest variation observed in the data.

FIGS. 13A-13E: cfChIP-seq identifies hepatocyte immune response in AIII plasma. (13A) Concept of statistical model. AIH samples with genes significantly above healthy baseline (left) are deconvoluted to their composing cell types. The gene signals are then compared to the expected signal based on the sample's cell-types composition using reference data and tested whether they are significantly above expected given the mean and variance of the expected pattern. (13B) Clustering of ˜600 genes shown in FIG. 11D. Left, cfChIP-seq actual values relative to healthy mean. Middle, expected levels based on the samples compositions. Right, residual signal (actual-expected) of samples. genes (rows) and samples (columns) are clustered by residual heatmap and order is identical in the three plots. (13C) Zoom in on genes that are significantly above expected in at least 3 AIH samples. (13D) Median and distribution of normalized promoter coverage shown in 13C in cfChIP-seq of AIH and healthy samples and ChIP-seq of liver tissue from reference atlas. (13E) Genome browser of view of selected genes from 13C, with HPX as a control for a liver specific gene and ACTB as a control gene.

FIGS. 14A-14D: plasma based classifier for AIH diagnosis. (14A) Heatmap showing patterns of the cfChIP-seq coverage in genes that are significantly elevated in the AIH samples compared to the non-AIH samples. Color scale represents the normalized coverage of the genomic region after log-transformation (log 2 (1+coverage)) and adjustment to zero mean for each gene across the samples. The undiagnosed category consists of samples collected from patients in the AIH clinic that are currently under observation due to elevated liver enzymes, and have not finally been diagnosed to date. (14B) Median and distribution of the AIH-score across all samples. The score (y-axis) indicates the difference in the cumulative converge of the AIH and non-AIH specific regions shown in 14A. (14C) AIH score (y-axis) as a function of liver percent (x-axis). cfChIP-seq samples (dots) are colored by group as in 14B and the red and blue lines indicate the linear fit of the AIH and non-AIH samples respectively. The diverging slopes demonstrate the ability of the AIH score to distinguish between AIH and non-AIH samples in plasma samples with liver contribution greater than 5%. (14D) ROC curve of AIH vs. non-AIH classification.

FIGS. 15A-15D: cfChIP-seq yield and healthy samples correlations. (15A) cfChIP-seq yield of AIH and healthy plasma samples. Values (y-axis) indicate the number of unique reads mapped to the genome (after duplicates removal). (15B) Evaluation of similarity of plasma samples from healthy adults (y-axis) and healthy pediatrics (x-axis). (15C) Pearson correlation of healthy pediatric samples, healthy adult samples and healthy adults vs. pediatrics samples. (15D) Frequency of genes with significantly elevated promoter coverage in AIH samples compared to healthy baseline shown in FIG. 1B.

FIGS. 16A-16B: tissue composition of AIH and healthy plasma samples. (16A) Tissue composition median and distribution of healthy and AIH samples. Same as FIG. 12A for all tissues. (16B) Scree plot of PCA plot shown in FIG. 2D.

FIGS. 17A-17D: residual cf-ChIP-seq signal unexplained by tissue composition. (17A) Statistical significance of the observed signal given the composition informed expectation of Refseq genes (rows) across healthy and AIH cfChIP-seq samples (columns). Color scale represents FDR corrected q-value (Methods). (17B) Patterns of promoter coverage of reference tissues and cell types (rows) over genes with elevated signal unexplained by tissue composition (columns). Color represents normalized coverage over the promoter region of the genes. (17C) Correlation of promoter coverage and estimated tissue fraction in the AIH samples. Gray background represents the correlation of all Refseq genes and the red histogram represents correlation of the 11 unexplained genes shown in 17A. (17D) Cumulative distribution function of correlation shown in 17C. black and red lines represent all Refseq genes and unexplained genes respectively.

FIGS. 18A-18B: Flowcharts of methods of (18A) tumor load estimation and (18B) marker region determination.

FIG. 19: A block diagram, depicting a computing device which may be included in a system for performing a method of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

The present invention, in some embodiments, provides methods of determining disease load or type in a subject suffering from a disease associated with cell death of a specific tissue or cell type. Methods of determining a cell free DNA chromatin immunoprecipitation and sequencing (cfChIP-Seq) marker are also provided as are methods of classifying a subject suffering from a disease, methods of classifying a subject as suffering from a specific small cell lung cancer (SCLC) subtype and methods of detecting autoimmune hepatitis (AIH) in a subject.

The invention is based, at least in part, on the surprising finding that cfChIP-seq recovers the unique epigenetic states of tissue and cell of origin, and importantly tumor gene expression, particularly of SCLC lineage-defining transcription factors and AIH related hepatocyte gene activity, providing a systematic view of disease state, and opening the possibility of molecularly classifying diseases directly from as little as 1 ml of plasma.

SCLC has a distinct cell-free chromatin signature, which can be detected in plasma of patients using cfChIP-seq and used to differentiate SCLC from other cancers and healthy controls. The SCLC signature can be detected using this approach even when it has low representation in the plasma (e.g., after therapy), and is highly correlated with serologic and radiological estimations of tumor burden and prognosis. In matched plasma and tumor biopsy samples, we show for the first time, the concordance of gene expression inferred from plasma cell-free chromatin and tumor transcriptome at the level of the individual patient. Importantly, cfChIP-seq profiles identify activity of key SCLC transcriptional drivers, including ASCL1 and NEUROD1 that drive NE phenotypes and POU2F3 that drives a non-NE phenotype. Furthermore, we identify signatures encompassing multiple genomic regions, which are consistent with gene expression changes in the relevant SCLC subtypes, that allow us to classify samples with a wide range of tumor contributions. These results set the stage for non-invasive subtyping and molecular profile-based treatments for patients with SCLC, which should be more effective than the current one-size fits all approach.

This study also provides a broadly applicable framework for benchmarking features of cfDNA against the tumor molecular profile or indeed disease molecular profile. While plasma cfChIP-seq shows good agreement with tumor RNA-seq, the correspondence is imperfect due to multiple factors. First, there are inherent differences between chromatin state, which is to a large extent on or off, and gene expression, which has a large dynamic range. In addition, several factors confound our estimations of both the plasma and tissue compartments. Plasma cell-free chromatin reflects contributions from multiple sources, which also includes the tumor. Tumor RNA-seq contains contributions from multiple sources in addition to the tumor including tissue-infiltrating immune cells, stromal cells, endothelial cells, and more. Thus, to understand the plasma-tumor correspondence, we need to account for the different cell-types contributing to each compartment. By explicitly accounting for differences in SCLC-score we could extract tumor-specific and tumor-extrinsic features even when the tumor contribution is low. Thus, cfChIP-Seq markers are distinct from RNA-seq markers and are uniquely useful for this assay.

We also made use of plasma cfChIP-seq to identify the transcription patterns of dying cells in AIH. We find that cfChIP-seq identifies elevated death of hepatocyte cells in patients with active disease. Moreover, applying a statistical model to explain away expected liver epigenetic landscape highlights abnormal transcription patterns taking place in the liver. Our control group is not intended for evaluating differential diagnosis and includes a wide variety of liver-related conditions beyond those relevant for that task. Systematic identification of genes that are specific for AIH and devising a robust classifier based on them requires a larger and more diverse control group. These examples, however, suggest the widespread applicability of cfChIP-seq for research of liver diseases and disease in general, and as a potential method for liquid biopsy in clinical setup and precision medicine.

Samples from AIH patients in remission show milder levels of liver-derived cfDNA, which is attributed to successful treatment. Importantly, in one case where there was discrepancy between liver enzyme levels and cfChIP-seq results, while liver enzymes were normal-both liver histology and cfChIP-seq results showed the patient had active disease. These results suggest that cfChIP-seq can be a valuable tool in monitoring the progression of the disease and optimizing treatment.

Overall, our study opens a unique opportunity to bridge the gap between molecular studies and patient treatment in particular in SCLC, an exceptionally lethal malignancy, which to date is treated as a homogenous disease with identical treatments for all patients and AIH. Moreover, this work identifies the applicability of cfChIP-seq to a wider context to profile and subtype tumors, in a way that can be transformative for patient care.

By a first aspect, there is provided a method of determining disease load in a subject, the method comprising:
a. receiving chromatin immunoprecipitation-sequencing (ChIP-Seq) reads from cell free DNA (cfDNA) from samples from:
   i. a first population of control subjects;
   ii. a second population of subjects suffering from the disease; and
   iii. the subject; and
b. assigning a disease load score to the subject based on the similarity of the subject's reads to reads from the second population and dissimilarity to reads from the first population;
thereby determining disease load in a subject.

In some embodiments, the method is an in vitro method. In some embodiments, the method is an ex vivo method. In some embodiments, the method is a diagnostic method. In some embodiments, the method is a prognostic method. As used herein, the term “disease load” refers to the number of disease cells or amount of disease in the body. In some embodiments, disease load is a measure of the relative or absolute amount of cfDNA derived from disease cells. In some embodiments, disease load is proportional to the relative or absolute amount of cfDNA derived from disease cells. In some embodiments, disease load is cancer load. In some embodiments, cancer load is tumor load. In some embodiments, the method is a liquid biopsy. In some embodiments, the method is a computerized method.

In some embodiments, the subject is a mammal. In some embodiments, the subject is a human. In some embodiments, the subject suffers from the disease. In some embodiments, the subject is in need of a method of the invention. In some embodiments, the subject suffers from cancer. In some embodiments, the subject is at risk of cancer. In some embodiments, the subject suffers from a liver disease. In some embodiments, the type of liver disease is not known. In some embodiments, the disease is associated with cell death. In some embodiments, the disease causes cell death. In some embodiments, the cell death is death of diseased cells. In some embodiments, the cell death is of a specific tissue. In some embodiments, the cell death is of a specific cell type. In some embodiments, the tissue or cell type is the diseased tissue or cell type. A skilled artisan would be aware of the diseases in which disease cells die off as part of the disease progression or pathology. As cell death results in release of cfDNA from the dead cells, any disease in which diseased cells die can be evaluated as part of the method of the invention.

In some embodiments, the disease is cancer. In some embodiments, the cancer is a solid cancer. In some embodiments, the cancer is a hematopoietic cancer. In some embodiments, the cancer is not a hematopoietic cancer. In some embodiments, the cancer is a tumor. In some embodiments, the cancer is selected from hepato-biliary cancer, cervical cancer, urogenital cancer (e.g., urothelial cancer), testicular cancer, prostate cancer, thyroid cancer, ovarian cancer, nervous system cancer, ocular cancer, lung cancer, soft tissue cancer, bone cancer, pancreatic cancer, bladder cancer, skin cancer, intestinal cancer, hepatic cancer, rectal cancer, colorectal cancer, esophageal cancer, gastric cancer, gastroesophageal cancer, breast cancer (e.g., triple negative breast cancer), renal cancer (e.g., renal carcinoma), skin cancer, head and neck cancer, leukemia and lymphoma. In some embodiments, the cancer is selected from hepato-biliary cancer, cervical cancer, urogenital cancer (e.g., urothelial cancer), testicular cancer, prostate cancer, thyroid cancer, ovarian cancer, nervous system cancer, ocular cancer, lung cancer, soft tissue cancer, bone cancer, pancreatic cancer, bladder cancer, skin cancer, intestinal cancer, hepatic cancer, rectal cancer, colorectal cancer, esophageal cancer, gastric cancer, gastroesophageal cancer, breast cancer (e.g., triple negative breast cancer), renal cancer (e.g., renal carcinoma), skin cancer, head and neck cancer.

In some embodiments, the cancer is lung cancer. In some embodiments, the lung cancer is small cell lung cancer (SCLC). In some embodiments, the cancer is a specific type of cancer. In some embodiments, the disease load score is a cancer load score. In some embodiments, the score is specific to the type of cancer. In some embodiments, the score is an SCLC score. In some embodiments, the score is for the specific type of cancer and not for other cancers. In some embodiments, the score is specific to the type of cancer. In some the score is for SCLC and not for other cancers. In some embodiments, the score is for SCLC and not for other types of lung cancer.

In some embodiments, the cancer is breast cancer. In some embodiments, the breast cancer is selected from Ductal carcinoma, Lobular carcinoma, Inflammatory breast cancer, and triple negative breast cancer. In some embodiments, the subtype of breast cancer is selected from Ductal carcinoma, Lobular carcinoma, Inflammatory breast cancer, and triple negative breast cancer. In some embodiments, the lung cancer is non-small cell lung cancer. In some embodiments, the non-small cell lung cancer is selected from Squamous cell carcinoma, Adenocarcinoma, and Large cell carcinoma. In some embodiments, the subtype of non-small cell lung cancer is selected from Squamous cell carcinoma, Adenocarcinoma, and Large cell carcinoma. In some embodiments, the cancer is prostate cancer. In some embodiments, the prostate cancer is selected from Adenocarcinoma, Small cell carcinoma, Ductal adenocarcinoma, and Prostatic intraepithelial neoplasia. In some embodiments, the subtype of prostate cancer is selected from Adenocarcinoma, Small cell carcinoma, Ductal adenocarcinoma, and Prostatic intraepithelial neoplasia. In some embodiments, the cancer is leukemia. In some embodiments, the leukemia is selected from Acute lymphoblastic leukemia (ALL), Chronic lymphocytic leukemia (CLL), Acute myeloid leukemia (AML) and Chronic myeloid leukemia (CML). In some embodiments, the subtype of leukemia is selected from Acute lymphoblastic leukemia (ALL), Chronic lymphocytic leukemia (CLL), Acute myeloid leukemia (AML) and Chronic myeloid leukemia (CML). In some embodiments, the cancer is lymphoma. In some embodiments, the lymphoma is selected from Hodgkin lymphoma, Non-Hodgkin lymphoma (NHL), Diffuse Large B-cell Lymphoma, Follicular Lymphoma, Mantle Cell Lymphoma, Burkitt Lymphoma, and Marginal Zone Lymphoma. In some embodiments, the subtype of lymphoma is selected from Hodgkin lymphoma, Non-Hodgkin lymphoma (NHL), Diffuse Large B-cell Lymphoma, Follicular Lymphoma, Mantle Cell Lymphoma, Burkitt Lymphoma, and Marginal Zone Lymphoma.

In some embodiments, the disease is liver disease. In some embodiments, the liver disease is characterized by death of liver cells. In some embodiments, liver cells are hepatocytes. In some embodiments, the liver disease is selected from autoimmune hepatitis (AIH), nonalcoholic steatohepatitis (NASH), fatty liver disease, cirrhosis of the liver, hepatitis B, hepatitis C, drug induced liver injury, Cholestatic liver disease, primary biliary cholangitis and liver cancer. In some embodiments, the liver disease is selected from autoimmune hepatitis (AIH), nonalcoholic steatohepatitis (NASH), fatty liver disease, cirrhosis of the liver, hepatitis B, hepatitis C, drug induced liver injury, Cholestatic liver disease, and primary biliary cholangitis. In some embodiments, the liver disease is AIH.

In some embodiments, the disease is a specific liver disease. In some embodiments, the disease load score is a liver disease load score. In some embodiments, the liver disease load score is specific to that liver disease and not to other liver diseases. In some embodiments, the liver disease load score is an AIH load score. In some embodiments, the score is specific to the liver disease. In some embodiments, the score is specific to AIH. In some embodiments, the score is specific to AIH and not to other liver diseases.

In some embodiments, the disease is an autoimmune disease. In some embodiments, the autoimmune disease causes cell death of cellular targets of the immune response. In some embodiments, the autoimmune disease causes cell death of targets of autoantibodies. In some embodiments, the autoimmune disease is selected from AIH, rheumatoid arthritis, multiple sclerosis (MS), diabetes, systemic sclerosis, psoriasis, coeliac disease, Alzheimer's disease, Parkinson's disease, lupus, autoimmune thyroid disease (e.g., Hashimoto's disease, Idiopathic thrombocytopeniasthenia gravis, Graves' disease, membranous nephropathy, pernicious anemia, inflammatory bowel disease (IBD) and Sjögren syndrome. In some embodiments, the autoimmune disease is AIH. In some embodiments, the autoimmune disease is diabetes. In some embodiments, diabetes is selected from Type I diabetes, Type II diabetes and gestational diabetes. In some embodiments, diabetes subtype is selected from Type I diabetes, Type II diabetes and gestational diabetes. In some embodiments, the autoimmune disease is Alzheimer's disease. In some embodiments, Alzheimer's disease is selected from early-onset Alzheimer's, late-onset Alzheimer's and familial Alzheimer's. In some embodiments, Alzheimer's disease subtype is selected from early-onset Alzheimer's, late-onset Alzheimer's and familial Alzheimer's. In some embodiments, the autoimmune disease is Parkinson's disease. In some embodiments, Parkinson's disease is selected from Idiopathic Parkinson's disease, Parkinson-plus syndromes (e.g., multiple system atrophy, progressive supranuclear palsy) and drug-induced Parkinson's disease. In some embodiments, Parkinson's disease subtype is selected from Idiopathic Parkinson's disease, Parkinson-plus syndromes (e.g., multiple system atrophy, progressive supranuclear palsy) and drug-induced Parkinson's disease. In some embodiments, the autoimmune disease is MS. In some embodiments, MS is selected from Relapsing-remitting multiple sclerosis (RRMS), Primary progressive multiple sclerosis (PPMS), and Secondary progressive multiple sclerosis (SPMS). In some embodiments, MS subtype is selected from Relapsing-remitting multiple sclerosis (RRMS), Primary progressive multiple sclerosis (PPMS), and Secondary progressive multiple sclerosis (SPMS). In some embodiments, the autoimmune disease is IBD. In some embodiments, IBD is selected from Crohn's disease and ulcerative colitis. In some embodiments, IBD subtype is selected from Crohn's disease and ulcerative colitis.

In some embodiments, the disease is a specific autoimmune disease. In some embodiments, the disease load score is an autoimmune disease load score. In some embodiments, the autoimmune load score is specific to that autoimmune disease and not to other autoimmune diseases. In some embodiments, the autoimmune disease load score is an AIH load score. In some embodiments, the score is specific to the autoimmune disease. In some embodiments, the score is specific to AIH. In some embodiments, the score is specific to AIH and not to other autoimmune diseases.

In some embodiments, the disease is a neurological disease. In some embodiments, a neurological disease is a neurodegenerative disease. In some embodiments, the target tissue is the brain. In some embodiments, a target cell is a neuron. In some embodiments, the disease load score is a brain or neuron load score. In some embodiments, a neurological disease is selected from Alzheimer's disease, Parkinson's disease, schizophrenia, depression and Huntington's disease. In some embodiments, the neurological disease is Alzheimer's disease. In some embodiments, the neurological disease is Parkinson's disease. In some embodiments, the neurological disease is schizophrenia. In some embodiments, the schizophrenia is selected from paranoid schizophrenia, disorganized schizophrenia and catatonic schizophrenia. In some embodiments, the schizophrenia subtype is selected from paranoid schizophrenia, disorganized schizophrenia and catatonic schizophrenia. In some embodiments, a subtype of neurological disease is selected from Alzheimer's disease, Parkinson's disease, schizophrenia, depression and Huntington's disease.

In some embodiments, the target tissue is the heart. In some embodiments, the target cells are cardiomyocytes. In some embodiments, the disease is cardiovascular disease. In some embodiments, cardiovascular disease is selected from Coronary artery disease, Hypertensive heart disease and congenital heart disease. In some embodiments, a cardiovascular disease subtype is selected from Coronary artery disease, Hypertensive heart disease and congenital heart disease.

In some embodiments, the target tissue is blood. In some embodiments, the target cells are blood cells. In some embodiments, blood cells are hematopoietic cells. In some embodiments, the disease is a blood disease or disorder. In some embodiments, a blood disease or disorder is selected from anemia, Iron-deficiency anemia, Vitamin B12 deficiency anemia, Folate deficiency anemia, Anemia of chronic disease, Hemolytic anemia, Sickle cell anemia, Thalassemia and Aplastic anemia. In some embodiments, a blood disease or disorder subtype is selected from anemia, Iron-deficiency anemia, Vitamin B12 deficiency anemia, Folate deficiency anemia, Anemia of chronic disease, Hemolytic anemia, Sickle cell anemia, Thalassemia and Aplastic anemia.

In some embodiments, the disease is an infection. In some embodiments, the infection is selected from a bacterial infection and a viral infection. In some embodiments, the infection subtype is selected from a bacterial infection and a viral infection. In some embodiments, the virus is human immunodeficiency virus (HIV/AIDS). In some embodiments, HIV is selected from HIV-1 and HIV-2. In some embodiments, the HIV subtype is selected from HIV-1 and HIV-2. In some embodiments, the target cells are immune cells. It is well known that many diseases have an immune component and that often the immune response is part of the pathology. Further, immune cells will often die as a result of many diseases and information on the disease state can be gleaned from the immune cells. In particular, the immune cells can be subtyped. In some embodiments, the subtype is an immune cell subtype. In some embodiments, the disease load is an immune cell load. In some embodiments, the immune cell load is a T cell load. In some embodiments, the target cell is a T cell. In some embodiments, the immune cell is a T cell. In some embodiments, the T cell is selected from CD8, CD4, Naïve T-cells, Effector T-cells, Th1 cells, Th2 cells, Th17 cells, Treg cells (Regulatory T-cells), Memory T-cells, Central memory T-cells, Effector memory T-cells, exhausted T-cells, and anergic T-cells. In some embodiments, the T cell subtype is selected from CD8, CD4, Naïve T-cells, Effector T-cells, Th1 cells, Th2 cells, Th17 cells, Treg cells (Regulatory T-cells), Memory T-cells, Central memory T-cells, Effector memory T-cells, exhausted T-cells, and anergic T-cells.

The tissue of cell type associated with each disease will be well known to a skilled artisan. For example, in lung cancer the tissue will be lung, just as in liver cancer the tissue will be liver. Further, in diabetes the tissue will be the pancreas and in multiple sclerosis the tissue will be muscle or nerve cells. The particular cell type within a tissue is also often known to a skilled artisan. For example, in liver the target cells may be hepatocytes, and in pancreas the target cells may be beta cells. Similarly, in myasthenia gravis the muscle cells maybe skeletal muscles expressing the acetylcholine receptor and in Graves' disease the cell type may be fibroblasts. Finally, in cancer the cell type may not be tissue dependent but rather an epithelial cell or a connective cell (e.g., a sarcoma).

In some embodiments, sequencing data is received. In some embodiments, sequencing is deep sequencing. In some embodiments, sequencing is next generation sequencing. In some embodiments, sequencing is massively parallel sequencing. In some embodiments, sequencing is whole genome sequencing. In some embodiments, sequencing is whole cfDNA sequencing. In some embodiments, sequencing is full exome sequencing. In some embodiments, the sequencing is sequencing of a sample.

In some embodiments, a sample is received. In some embodiments, the sample is from the subjects. In some embodiments, each subject of a population provides a sample. In some embodiments, a sample is from each subject of the first population, each subject of the second population and the subject. In some embodiments, the method comprises extracting the sample. In some embodiments, the ChIP-Seq reads are from a sample. In some embodiments, the sample comprises cfDNA. In some embodiments, the sample is a bodily fluid sample. In some embodiments, the bodily fluid is selected from at least one of: blood, plasma, gastric fluid, intestinal fluid, saliva, bile, tumor fluid, breast milk, urine, vaginal fluid, interstitial fluid, cerebral spinal fluid and stool. In some embodiments, the bodily fluid is blood. In some embodiments, the sample is a blood sample. In some embodiments, blood is whole blood. In some embodiments, blood is plasma. In some embodiments, the receiving comprises receiving a sample and performing ChIP-Seq on cfDNA from the sample.

In some embodiments, the ChIP-Seq reads are from at least one location in the genome. In some embodiments, at least one is a plurality. In some embodiments, at least one location is in a first gene body or gene promoter and at least one location is in a second gene body or gene promoter wherein the first and second gene are not the same gene. In some embodiments, reads from a plurality of genes are provided. In some embodiments, reads from a plurality of locations are provided. In some embodiments, at least one is at least 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950 or 1000 locations. Each possibility represents a separate embodiment of the invention. In some embodiments, at least one is at least 10. In some embodiments, at least one is at least 50. In some embodiments, at least one is at least 100. In some embodiments, all reads within a gene body or gene promoter are provided. In some embodiments, all reads within a gene body or gene promoter are analyzed. In some embodiments, all reads from the cfDNA are provided. In some embodiments, all reads from the cfDNA are analyzed.

Methods of ChIP-Seq are well known in the art and any such method may be used. In particular, methods of ChIP-Seq are provided hereinbelow. In some embodiments, the ChIP-Seq is performed on cfDNA. In some embodiments, the method does not comprise lysing cells. In some embodiments, intact cells are removed before performing ChIP-Seq. In some embodiments, the ChIP is performed in the sample. In some embodiments, the ChIP is performed in the blood. In some embodiments, the method does not comprise isolating cfDNA. In some embodiments, the method comprises isolating cfDNA from the sample. Standard techniques for cell-free DNA extraction are known to a skilled artisan, a non-limiting example of which is the QIAamp Circulating Nucleic Acid kit (QIAGEN).

In some embodiments, the ChIP-Seq comprises chromatin immunoprecipitation followed by sequencing. In some embodiments, ChIP comprises contacting a sample with at least one reagent that binds to a DNA-associated protein. As used herein, “a reagent that binds” refers to any protein binding molecule or composition. Protein binding is well known in the art and may be assessed by any assay known in the art, including but not limited to yeast-2-hybrid, immunoprecipitation, competition assay, phage display, tandem affinity purification, and proximity ligation assay. In some embodiments, the reagent is a proteinaceous molecule. In some embodiments, the reagent is selected from an antibody or antigen binding fragment thereof, a protein and a small molecule. Small molecules that bind to specific proteins are well known in the art and may be used for pull-down experiments. Additionally, well characterized protein-protein interactions may be used for pull-downs. Indeed, any reagent that may be used for precipitation, immunoprecipitation (IP) or chromatin immunoprecipitation (ChIP), may be used as the reagent. In some embodiments, the reagent is an antibody or antigen binding fragment thereof.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgA1 and IgA2) or subclass.

In some embodiments, one reagent is contacted. In some embodiments, at least one reagent is contacted. In some embodiments, more than one reagent is contacted. In some embodiments, each reagent binds a different DNA-associated protein. In some embodiments, each reagent binds a different histone. In some embodiments, each reagent binds a different histone modification. In some embodiments, modification is modification of the histone tail.

In some embodiments, the reagent is conjugated to a physical support. As used herein, the term “physical support” refers to a solid and stable molecule that gives support to the reagent. In some embodiments, the support is a scaffold or scaffolding agent. In some embodiments, the support is a resin. In some embodiments, the support is a bead. In some embodiments, the support is a magnetic or paramagnetic bead. Magnetic beads may be purchased for examples from Dynabeads or Pierce. In some embodiments, the support is an agarose bead. In some embodiments, the support is a Sepharose bead. In some embodiments, the support is an artificial support. In some embodiments, the support is a protein A/G bead. In some embodiments, the reagent is conjugated to the physical support before the contacting. In some embodiments, the reagent is conjugated to the physical support before the ChIP. In some embodiments, the conjugating is a covalent linkage. In some embodiments, the conjugating is by epoxy chemistry. In some embodiments, the support aids in isolation of the reagent, wherein the isolating is isolating the physical support. In some embodiments, an adapter is added to the cfDNA while it is on the physical support. In some embodiments, adapter ligation is done on physical support. In some embodiments, on physical support is on bead. In some embodiments, the isolating is isolating adapter ligated cfDNA.

As used herein, the term “DNA-associated protein” refers to any protein that can be precipitated with DNA or when precipitated brings along DNA. In some embodiments, the DNA-associated protein directly binds DNA. In some embodiments, the DNA-associated protein is a component of chromatin. In some embodiments, the DNA-associated protein binds-indirectly to DNA. In some embodiments, the DNA-associated protein binds to genomic DNA. In some embodiments, the DNA-binding protein binds in the promoter. In some embodiments, the DNA-binding protein binds in a gene body. In some embodiments, the DNA-binding protein binds to a cis or trans regulatory element. In some embodiments, the DNA-associated protein is associated with transcription. In some embodiments, the DNA-associated protein marks transcription. In some embodiments, transcription is active transcription. In some embodiments, the DNA-associated protein marks repressed transcription.

In some embodiments, the DNA-associated protein binds DNA and is a non-sequence specific DNA binder. In some embodiments, the DNA-associated protein binds DNA is a sequence specific DNA binder or a non-sequence specific DNA binder. Examples of non-sequence specific DNA binders include histones, high-mobility group (HMG) proteins, members of the DNA damage repair machinery and members of the general transcriptional machinery. The general transcriptional machinery is well defined and includes, but is not limited to, RNA polymerases, DNA helicases, general cofactors, the splicing machinery and the polyA machinery. The DNA damage repair machinery is also well defined and includes, but is not limited to, members of the nucleotide excision repair pathway, base excision repair pathway and the mismatch repair system. In some embodiments, the DNA-associated protein is a modified protein. In some embodiments, the modification is a post-translational modification. In some embodiments, the reagent binds to the modified form of the protein. In some embodiments, the reagent binds only or predominantly to the modified form of the protein.

In some embodiments, the DNA-associated protein is a histone, modified histone or histone variant. Modifications to the histone tail are well known in the art, and include but are not limited to methylation, acetylation, sumoylation, ubiquitylation and phosphorylation. Modifications may be multiple such as tri-methylation or poly-ubiquitylation. In some embodiments, a tail may have multiple modifications such as methylation and phosphorylation. The histone may be one of the core histones, H1, H2A, H2B, H3 and H4, or it may be a histone variant such as, for non-limiting example, H2A.z, gammaH2AX, H1T, and H3.3. In some embodiments, the modified or variant histone has an activating function on transcription. In some embodiments, the modified or variant contributes to the formation of euchromatin. In some embodiments, the modified or variant contributes to the formation of heterochromatin. In some embodiments, the histone or variant histone is associated with transcription. In some embodiments, associated with transcription is marks transcription. In some embodiments, associated with transcription is marks repressed transcription. In some embodiments, transcription is active transcription.

In some embodiments, the modified histone is selected from.

In some embodiments, the modified histone is selected from histone H3 lysine 79 monomethylation (H3K79me), histone H3 lysine 79 dimethylation (H3K79me2), histone H3 lysine 79 trimethylation (H3K79me3), histone H3 serine 10 phosphorylation (H3S10ph), histone H3 arginine 17 monomethylation (H3R17me), histone H4 arginine 3 monomethylation (H4R3me), histone H2B ubiquitination (H2Bub), histone H3 lysine 4 trimethylation (H3K4me3), histone H3 lysine 27 acetylation (H3K27Ac), histone H3 lysine 36 monomethylation (H3K36me), histone H3 lysine 36 dimethylation (H3K36me2), histone H3 lysine 36 trimethylation (H3K36me3), histone H3 lysine 4 acetylation (H3K4ac), histone H3 lysine 9 acetylation (H3K9ac), histone H3 lysine 14 acetylation (H3K14ac), histone H3 lysine 18 acetylation (H3K18ac), histone H3 lysine 23 acetylation (H3K23ac), histone H3 lysine 36 acetylation (H3K36ac), histone 3 lysine 56 acetylation (H3K56ac), histone H4 lysine 8 acetylation (H4K8ac), histone H4 lysine 12 acetylation (H4K12ac), histone H4 lysine 16 acetylation (H4K16ac), histone H4 lysine 20 acetylation (H4K20ac), histone H4 lysine 59 acetylation (H4K59ac), histone 4 lysine 91 acetylation (H4K91ac), histone H2A lysine 5 acetylation (H2AK5ac), histone H2B lysine 5 acetylation (H2BK5ac), histone H2B lysine 12 acetylation (H2BK12ac), histone H2B lysine 15 acetylation (H2BK15ac), histone H2B lysine 20 acetylation (H2BK20ac), histone H3 lysine 4 monomethylation (H3K4me), and histone H3 lysine 4 dimethylation (H3K4me2). In some embodiments, acetylation refers to any modification with an acyl group. In some embodiments, acetylation refers to any modification with an acyl group that can be covalently attached to a lysine on a histone tail. In some embodiments, a modification with an acyl group is selected from an acetyl group, a crotonyl group, a propionyl group and a butyryl group. In some embodiments, a modification with an acyl group is selected from a crotonyl group, a propionyl group and a butyryl group. In some embodiments, a modified histone that is associated with transcription is selected from H3K4me3, H3K27Ac, H3K36me3, H3K4me, and H3K4me2. In some embodiments, a modified histone that is associated with transcription is selected from H3K4me, H3K4me2, H3K4me3, H3K36me, H3K36me2, H3K36me3, H3K79me, H3K79me2, H3K79me3, H3S10ph, H3R17me, H4R3me, H2Bub, H3K4ac, H3K9ac, H3K14ac, H3K18ac, H3K23ac, H3K27ac, H3K36ac, H3K56ac, H4K5ac, H4K8ac, H4K12ac, H4K16ac, H4K20ac, H4K59ac, H4K91ac, H2AK5ac, H2BK5ac, H2BK12ac, H2BK15ac, and H2BK20ac. In some embodiments, the DNA associate protein is H3K4me3. In some embodiments, the modified histone associated with transcription is H3K4me3.

In some embodiments, the modified histone is selected from histone 3 lysine 27 trimethylation (H3K27me3), histone 3 lysine 27 dimethylation (H3K27me2), histone 3 lysine 27 monomethylation (H3K27me), histone 3 lysine 9 monomethylation (H3K9me), histone H3 arginine 2 monomethylation (H3R2me), histone H4 lysine 20 trimethylation (H4K20me3), histone H2A ubiquitination (H2Aub), histone H2A serine 1 phosphorylation (H2AS1ph) and histone 3 lysine 9 trimethylation (H3K9me3). In some embodiments, the modified histone that is associated with repressed transcription is selected from H3K27me3, H3K27me2, H3K27me, H3K9me, H3R2me, H3K27me2, H3K27me3, H4K20me3, H2Aub, H2AS1ph, and H3K9me3. In some embodiments, the modified histone that is associated with repressed transcription is selected from H3R2me, H3K9me, H3K9me2, H3K9me3, H3K27me, H3K27me2, H3K27me3, H4K20me3, H2Aub, H2AS1ph.

In some embodiments, ChIP further comprises isolating the reagent. In some embodiments, isolating the reagent is isolating the support. In some embodiments, the isolating isolates any proteins bound to the reagent. In some embodiments, the isolating isolates any DNA bound to the protein. In some embodiments, the isolating isolates any DNA bound to the reagent. In some embodiments, the DNA is cfDNA.

In some embodiment, the ChIP-Seq further comprises sequencing the isolated cfDNA. In some embodiments, the ChIP-Seq further comprises isolating the cfDNA from the reagent. In some embodiments, the ChIP-seq further comprises isolating the cfDNA from the support. In some embodiments, the isolating is eluting. In some embodiments, the cfDNA is adapter ligated cfDNA. In some embodiments, the adapter is a sequencing adapter. In some embodiments, the adapter is added on bead. In some embodiments, the adapter is added in the bodily fluid. In some embodiments, the adapter is added after isolation of the reagent. In some embodiments, the adapter is added off bead.

In some embodiments, a disease load score is assigned to the subject. In some embodiments, the score is proportional to the disease load in the subject. In some embodiments, score is based on the similarity of the subject's reads to the reads from the second population. In some embodiments, the score is based on the similarity of the subject's reads to the reads from the first population. In some embodiments, similarity is dissimilarity. Methods of determining similarity are well known in the art and any such method may be used. In some embodiments, the similarity is determined by linear regression analysis. In some embodiments, the similarity is determined by a trained machine learning algorithm.

In some embodiments, the machine learning algorithm is trained on the ChIP-Seq reads from the first population and the second population. In some embodiments, the machine learning algorithm is trained on labels identifying the ChIP-Seq reads as being from a control subject or a disease subject. In some embodiments, the machine learning algorithm is trained on labels identifying the ChIP-Seq reads as being from a subject of the first population or a subject of the second population. In some embodiments, each set of ChIP-Seq reads from a subject has a corresponding label. In some embodiments, each subject has a corresponding label. In some embodiments, the machine learning algorithm is a classifier.

Machine learning models are well known in the art and any such model may be used. Models include, but are not limited to artificial neural networks, support vector machines (SVM) classifier and a k-nearest neighbor (k-NN) classifier. In some embodiments, the machine learning model is a classification model. In some embodiments, the machine learning model is a classifier. In some embodiments, the machine learning model is an SVM classifier. In some embodiments, the machine learning model is a k-NN classifier. In some embodiments, the machine learning model is selected from an SVM classifier and a k-NN classifier. In some embodiments, the algorithm is a boosting algorithm. In some embodiments, the ML model employs the algorithm. In some embodiments, the ML model is the algorithm. In some embodiments, the algorithm is a random forest algorithm. In some embodiments, a machine learning model implements a machine learning algorithm. In some embodiments, the machine learning model is a supervised model. In some embodiments, supervised is self-supervised. In some embodiments, the machine learning algorithm outputs the score.

In some embodiments, the control subjects are healthy subjects. In some embodiments, the controls are healthy controls. In some embodiments, the control subjects suffer from a different disease than the subject suffers from. In some embodiments, the control subjects suffer from a disease of the same tissue or cell type as the subject, but from a different disease. In some embodiments, the subject and the control subjects suffer from cancer but different types of cancer. In some embodiments, the subject and the control subjects suffer from a liver disease but different liver diseases. In some embodiments, the subject and the control subjects suffer from an autoimmune disease but different autoimmune diseases.

In some embodiments, the control is a composite of cell types. In some embodiments, the composite comprises the cell types found in a healthy sample. In some embodiments, the cell types are found in the relative abundance found in a healthy sample. In some embodiments, the composite comprises the cell types found in a subject suffering from a disease. In some embodiments, the composite comprises the cell types found in the subjects of the second population. In some embodiments, found in is found in a sample. In some embodiments, the cell types are found in the relative abundance found in a subject suffering from the disease. In some embodiments, the cell types are found in the relative abundance found in the subjects of the second population.

In some embodiments, the method further comprises determining the relative abundance of cell types in a subject suffering from the disease. In some embodiments, the method further comprises determining the relative abundance of cell types in a subject of the second population. In some embodiments, a subject is each subject. In some embodiments, a subject is all subjects. In some embodiments, the ChIP-Seq reads are deconvoluted to determine the relative abundance of cell types. In some embodiments, a control composite is generated. In some embodiments, a control composite comprises control ChIP-Seq reads from healthy cell types combined. In some embodiments, the ChIP-Seq reads are combined in a relative proportion that is the same as the relative proportion of those cell types in a ChIP-Seq reads from a subject suffering from the disease. In some embodiments, the ChIP-Seq reads are combined in a relative proportion that is the same as the relative proportion of those cell types in a ChIP-Seq reads from a subject of the second population. In some embodiments, a subject of the second population is the average of the subjects of the population.

In some embodiments, the subjects of the second population suffer from the same disease as the subject. In some embodiments, the same disease is the same type of disease. In some embodiments, the same disease is the same cancer. In some embodiments, the same disease is the same liver disease. In some embodiments, the same disease is the same autoimmune disease. In some embodiments, the diagnosis of the subjects of the second population is known. In some embodiments, the gene expression profile of the disease in the subjects of the second population is known. In some embodiments, RNA-Seq has been performed on disease cells from the subjects of the second population. In some embodiments, RNA-Seq is performed on a biopsy. In some embodiments, the biopsy is a tumor biopsy. In some embodiments, the biopsy is a liver biopsy.

In some embodiments, the control subjects are healthy subjects and the second population is a subset of subjects that suffer from the same disease as the subject. In some embodiments, the subset is a subset of the second population. In some embodiments, the subset comprises the subjects with the most differentially expressed genes as compared to the first population. In some embodiments, the subset comprises the subjects with the most differential signal compared to the first population. In some embodiments, the differential signal is from regions in gene promoters. In some embodiments, the differential signal is from regions in gene bodies. In some embodiments, the differential signal is from regions in intergenic regions. In some embodiments, the differential signal is based on RNA-Seq data. In some embodiments, the differential signal is based on ChIP-Seq reads. In some embodiments, the differential signal comprises differential reads in an informative genomic locus in a gene. In some embodiments, the most is the top 1, 2, 3, 4, 5, 6, 7, 8, 9, 10, 11, 12, 13, 14, 15, 16, 17, 18, 19, 20, 21, 22, 23, 24 or 25%. Each possibility represents a separate embodiment of the invention. In some embodiments, the most is the top 10%. In some embodiments, the most is the top 20%. In some embodiments, the most is those with a number of differentially expressed genes beyond a predetermined threshold. In some embodiments, the subset is the architype of the second population. In some embodiments, the subset is the architype of the disease. In some embodiments, the architype is made up of the disease subjects that are most dissimilar to the healthy controls.

In some embodiments, the method is a method of measuring disease load. In some embodiments, disease load is proportional to the amount of disease present. In some embodiments, a disease load above a predetermined threshold indicates the disease is present in the subject. In some embodiments, the predetermined threshold is zero. In some embodiments, a score above a predetermined threshold indicates the subject suffers from the disease. In some embodiments, a score above a predetermined threshold indicates the subject suffers from active disease. In some embodiments, the method is a method of diagnosing a disease. In some embodiments, the disease is the disease of the subjects of the second population. In some embodiments, a score above a predetermined threshold indicates the subject suffers from the specific disease of the subjects of the second population. In some embodiments, a score above a predetermined threshold indicates the subject suffers from SCLC. In some embodiments, a score above a predetermined threshold indicates the subject suffers from AIH.

By another aspect, there is provided a method of determining a ChIP-Seq marker that distinguishes cfDNA from a first disease from cfDNA from a second disease, the method comprising:
a. determining disease load in at least one subject suffering from the first disease; and
b. comparing ChIP-Seq reads from the at least one subject with ChIP-Seq reads from a third population of subjects suffering from the second disease and selecting at least one genomic region with a differential signal between the at least one subject and the third population;
thereby determining a ChIP-Seq marker.

In some embodiments, the method is a method of determining markers for a disease. In some embodiments, the method is a method of determining markers for a disease subtype. In some embodiments, the disease subtype is a cancer subtype. In some embodiments, the first disease and the second disease are the same type of disease. In some embodiments, the first disease and the second disease of diseases of the same tissue. In some embodiments, the first disease and the second disease are diseases of the same cell type. In some embodiments, the marker distinguishes between subtypes of a class of diseases to which the first disease and second disease both belong. In some embodiments, the first disease and second disease are both cancer. In some embodiments, the first disease and second disease are lung cancer. In some embodiments, the first disease and second disease are both SCLC. In some embodiments, the first disease and the second disease are both liver diseases. In some embodiments, the first disease and the second disease are both autoimmune diseases. In some embodiments, the method is a method of determining a marker for a SCLC subtype. In some embodiments, the method is a method of determining a marker for AIH.

In some embodiments, determining a marker is determining at least one marker. In some embodiments, at least marker is a plurality of markers. In some embodiments, a plurality of markers is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 markers. Each possibility represents a separate embodiment of the invention. In some embodiments, a marker is a genomic location. In some embodiments, a marker is a genomic locus. In some embodiments, the genomic locus is an informative genomic locus.

As used herein, the terms “informative genomic location” and “informative genetic locus” are used synonymously and refer to a unique DNA sequence in a particular location in the genome that when associated with a given DNA-associated protein is informative of the disease in which the association occurs. In some embodiments, active transcription at the genomic location is informative of the disease. In some embodiments, repressed transcription at the genomic location is informative of the disease. In some embodiments, it is informative of the disease that killed the cell in which the association occurs. In some embodiments, the location is a tissue or cell type specific binding/association site. In some embodiments, the binding/association is not specific/unique, but highly enriched in the tissue or cell type. In some embodiments, it is informative of the cellular state of the cell in which the association occurs. In some embodiments, it is informative of both the tissue of origin and/or cell type and the cellular state of the cell in which the association occurs. In some embodiments, it is informative of a disease in the cell. In some embodiments, it is informative of a transcriptional program in the cell. In some embodiments, it is informative of the subtype of the disease.

In some embodiments, disease load is determined with a first population that are healthy subjects. In some embodiments, disease load is determined with a first population that are subjects that suffer from a different disease than the first disease. In some embodiments, the different disease is the second disease. In some embodiments, the different disease is not the second disease. In some embodiments, the different disease is a third disease.

In some embodiments, disease load is determined in at least one subject suffering from the first disease. In some embodiments, at least one is a plurality. In some embodiments, at least one is at least 1, 2, 3, 4, 5, 6, 7, 8, 9, 10, 12, 15, 17, 20, 22, 25, 27, 30, 32, 35, 37, 40, 45, 50, 55, 60, 65, 70, 75, 80, 85, 90, 95 or 100 subjects. Each possibility represents a separate embodiment of the invention. In some embodiments, the method further comprises a step of selecting a subset of the at least one subjects. In some embodiments, the selecting is after the determining disease load. In some embodiments, the selecting is before the comparing. In some embodiments, a subset of the plurality is selected. In some embodiments, the subset is a subset with a disease load above a predetermined threshold. In some embodiments, the subset is the subjects with the highest disease load. In some embodiments, the highest disease load is the top, 1, 2, 3, 4, 5, 6, 7, 8, 9, 10, 11, 12, 13, 14, 15, 16, 17, 18, 19, 20 or 25% of subjects. Each possibility represents a separate embodiment of the invention. In some embodiments, the highest disease load is the top 10% of subjects.

In some embodiments, comparing ChIP-Seq reads comprises comparing ChIP-Seq reads from the subset with the third population. In some embodiments, the ChIP-Seq reads are from a sample. In some embodiments, the ChIP-Seq reads are from cfDNA. In some embodiments, the cfDNA is from the sample. In some embodiments, the method further comprises performing ChIP-Seq. In some embodiments, the ChIP-Seq reads are from genomic regions with a differential signal between the first population and the second population. It will be understood by a skilled artisan that the reads which are interrogated in order to find a marker can be a subset of all the reads from the cfDNA. This subset can be only the genomic regions which have a differential signal between the first population and the second population. This narrows the field to examiner and increases the likelihood of finding a useful marker.

In some embodiments, at least one genomic region is selected. In some embodiments, at least one is a plurality. In some embodiments, all genomic regions with differential signal are selected. In some embodiments, differential signal is a signal with a significant difference. In some embodiments, significant is statistically significant. In some embodiments, a signal is the number of reads in the region. In some embodiments, the number of reads is the cumulative number of reads.

In some embodiments, a region is a window. In some embodiments, a region comprises or consists of at least 40, 50, 75, 100, 150, 200, 250, 300, 350, 400, 450, 500, 600, 650, 700, 750, 800, 850, 900, 950, 1000, 1100, 1200, 1300, 1400, 1500, 1600, 1700, 1800, 1900 or 2000 bases. Each possibility represents a separate embodiment of the invention. In some embodiments, a region comprises or consists of at least 200 bases. In some embodiments, a region is at most 600, 650, 700, 750, 800, 850, 900, 950, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500 or 10000 bases. Each possibility represents a separate embodiment of the invention. In some embodiments, a region comprises or consists of at most 6000 bases. In some embodiments, a region comprises or consists of at most 5000 bases. In some embodiments, a region comprises or consists of at most 1000 bases. In some embodiments, a region comprises or consists of between 200 and 6000 bases. In some embodiments, a region comprises or consists of between 200 and 1000 bases. In some embodiments, a region is not the entire genome. In some embodiments, a region is not an entire chromosome. In some embodiments, region is within a gene body. In some embodiments, a region is outside of a gene body. In some embodiments, a region is in an intergenic area. In some embodiments, a region is within a regulatory element of a gene. In some embodiments, the regulatory element is a promoter. In some embodiments, a region is around a transcriptional start site of a gene. In some embodiments, a region is within a regulatory element of a gene and that gene's gene body. In some embodiments, around is within 2 kb. In some embodiments, a region is within a regulatory element of a gene and that gene's gene body. In some embodiments, around is within 3 kb. In some embodiments, around is upstream. In some embodiments, around is downstream. In some embodiments, around is both upstream and downstream.

In some embodiments, the gene is a transcription factor. In some embodiments, the gene is a transcriptional coregulator. In some embodiments, expression of the gene is indicative of the disease. In some embodiments, expression of the gene is indicative of the subtype. In some embodiments, expression of a plurality of the markers is indicative of the disease. In some embodiments, expression of a plurality of the markers is indicative of the subtype.

By another aspect, there is provide a method of classifying a subject as suffering from a first disease, the method comprising:
a. receiving ChIP-Seq reads from the subject; and
b. identifying reads from a ChIP-Seq marker that differentiates the first disease from a second disease, wherein ChIP-Seq reads above a predetermined threshold indicate the subject suffers from the first disease;
thereby classifying a subject as suffering from a first disease.

In some embodiments, the ChIP-Seq marker is determined by a method of the invention. In some embodiments, the method further comprises determining a ChIP-Seq marker for the first disease. In some embodiments, the determining is before the identifying. In some embodiments, the determining is before the receiving. In some embodiments, the marker for a first disease distinguishes the first disease from a second disease. In some embodiments, a second disease is all other diseases. In some embodiments, all other diseases are all other diseases of a known type. In some embodiments, all other diseases are all other diseases of a specific tissue or cell type. In some embodiments, suffering from the first disease is not suffering from the second disease.

In some embodiments, the method further comprises administering to the subject a therapeutic agent that treats the disease. In some embodiments, the method further comprises administering to the subject a therapeutic agent that treats the first disease. In some embodiments, the agent treats the first disease and not the second disease. In some embodiments, the therapeutic agent is the first line treatment for the disease. In some embodiments, the disease is the first disease. In some embodiments, agent is specific to the first disease. In some embodiments, the agent is approved for treating the first disease and not the second. In some embodiments, the method further comprises administering to the subject a therapeutic agent that treats the subtype of the disease. In some embodiments, the administering is to a subject determined to suffer from the disease. In some embodiments, the administering is to a subject determined to suffer from the subtype. In some embodiments, the agent treats SCLC. In some embodiments, the agent treats AIH.

By another aspect, there is provided a method of assigning a subject suffering from lung cancer to a SCLC subtype, the method comprising:
a. receiving ChIP-Seq reads from the subject; and
b. identifying reads from at least one informative genomic locus as being beyond a predetermined threshold;
thereby assigning a subject suffering from lung cancer to a SCLC subtype.

By another aspect, there is provided a method of detecting AIH in a subject, the method comprising:
a. receiving ChIP-Seq reads from the subject; and
b. identifying reads from at least one informative genomic locus as being beyond a predetermined threshold;
thereby detecting AIH in a subject.

In some embodiments, the subject suffers from SCLC. In some embodiments, the subtypes are selected from a neuroendocrine subtype and a non-neuroendocrine subtype. In some embodiments, the subtypes are selected from: achaete-scute homolog 1 (ASCL1) subtype, neurogenic differentiation 1 (NEUROD1) subtype, POU domain class 2 transcription factor 3 (POU2F3) subtype, yes-associated protein 1 (YAP1) subtype and protein atonal homolog 1 (ATOH1) subtype. SCLC subtypes are well known to be classified by the transcription factors active in that subtype. In some embodiments, SCLC is of a neuroendocrine subtype or a non-neuroendocrine subtype. In some embodiments, a neuroendocrine subtype is a neuroendocrine high subtype. In some embodiments, a non-neuroendocrine subtype is a non- or low-neuroendocrine subtype. In some embodiments, the ASCL1 subtype is a neuroendocrine subtype. In some embodiments, a NEUROD1 subtype is a neuroendocrine subtype. In some embodiments, a POU2F3 subtype is a non-neuroendocrine subtype. In some embodiments, a YAP1 subtype is a non-neuroendocrine subtype. In some embodiments, an ATOH1 subtype is a non-neuroendocrine subtype. In some embodiments, the subtype is selected from: ASCL1, NEUROD1, POU2F3 and ATOH1 subtypes. In some embodiments, the subtype is selected from: ASCL1, NEUROD1, POU2F3 and YAP1 subtypes. In some embodiments, the subtype is selected from: ASCL1, NEUROD1, and POU2F3 subtypes.

In some embodiments, the informative genomic locus is a marker. In some embodiments, the informative genomic locus is one determined by a method of the invention. In some embodiments, the informative genomic locus is selected from the locations provided in Tables 1-3. In some embodiments, the informative genomic locus is selected from the locations provided in Table 1. In some embodiments, the informative genomic locus is selected from the locations provided in Table 2. In some embodiments, the informative genomic locus is selected from the locations provided in Table 3.

In some embodiments, the informative genomic locus is selected from the locations provided in Table 1 and reads above a predetermined threshold indicates the subject suffers from ASCL1 subtype of SCLC. In some embodiments, the informative genomic locus is selected from the locations provided in Table 2 and reads above a predetermined threshold indicates the subject suffers from NEUROD1 subtype of SCLC. In some embodiments, the informative genomic locus is selected from the locations provided in Table 3 and reads above a predetermined threshold indicates the subject suffers from POU2F3 subtype of SCLC. In some embodiments, beyond is above. In some embodiments, the informative genomic locus is at least three genomic loci wherein at least one is selected from the locations provided in Table 1, at least one is provided in Table 2 and at least one is provided in Table 3 and reads below a predetermined threshold at each of the at least three genomic loci indicate the subject suffers from ATOH1 or YAP1 subtype of SCLC. In some embodiments, ATOH1 or YAP1 is ATOH1. In some embodiments, ATOH1 or YAP1 is YAP1. In some embodiments, beyond is below. In some embodiments, beyond is above or below.

In some embodiments, at least one informative genomic locus is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 loci. Each possibility represents a separate embodiment of the invention. In some embodiments, at least one is a plurality of genomic loci. In some embodiments, a plurality of genomic loci is examined and if reads above a predetermined threshold are identified at any of the loci the subtype is determined. In some embodiments, a plurality of genomic loci is examined and if reads above a predetermined threshold are identified at any of the loci AIH is detected. In some embodiments, at least one locus from a Table is all the loci in the Table. In some embodiments, all loci from a Table are all statistically significant loci from a Table. In some embodiments, at least one locus from a Table is at least one statistically significant loci from the Table. In some embodiments, statistically significant loci from Table 1 are loci numbers 1-38 from Table 1. In some embodiments, statistically significant loci from Table 2 are loci numbers 1-8 from Table 2. In some embodiments, statistically significant loci from Table 3 are loci numbers 1-30 from Table 3.

In some embodiments, an informative genomic locus for AIH is selected from a genomic locus within a gene selected from: BCL2L14, CXCL10, CXCL11, CXCL9, GBP1, GBP5, HAPLN3, HLA-DOB, IL32, KB-1615E4.2, MARVELD3, OAS2, TRIM31, UBD, UPP2. In some embodiments, an informative genomic locus for AIH is selected from a promoter region within a gene selected from: BCL2L14, CXCL10, CXCL11, CXCL9, GBP1, GBP5, HAPLN3, HLA-DOB, IL32, KB-1615E4.2, MARVELD3, OAS2, TRIM31, UBD, UPP2. In some embodiments, the promoter comprises or consists of a region 2 kb upstream of a transcriptional start site of the gene. In some embodiments, the promoter comprises or consists of a region 1 kb upstream of a transcriptional start stie of the gene. In some embodiments, the promoter comprises or consists of a region 1 kb downstream of a transcriptional start site of the gene. In some embodiments, the promoter comprises or consists of a region from 1 kb upstream to 1 kb downstream of the a transcriptional start stie of the gene.

In some embodiments, determining a cancer subtype correlates with predicted subject survival time. In some embodiments, determining a cancer subtype comprises determining subject survival time. In some embodiments, the method is a method of predicting subject survival time. In some embodiments, detecting AIH comprises diagnosing AIH. In some embodiments, detecting AIH comprises prognosing AIH. In some embodiments, diagnosing AIH comprises determining a subject heretofore not known to suffer from AIH does suffer from AIH. In some embodiments, detecting AIH is monitoring AIH after treatment. In some embodiments, detecting AIH is detecting residual disease. In some embodiments, the method is a method of monitoring AIH in a subject being administered a treatment for AIH. In some embodiments, monitoring comprises detecting a change in AIH load. In some embodiments, the change is a reduction.

In some embodiments, the method further comprises administering to the subject at least one therapeutic agent that treats the SCLC subtype. In some embodiments, the agent is specific to the subtype. In some embodiments, the agent is approved from the subject. In some embodiments, specific to the subtype is not indicated for a different subtype. In some embodiments, subtype is the neuroendocrine subtype and the therapeutic agent comprises chemotherapy. In some embodiments, the subtype is non-neuroendocrine subtype and the therapeutic agent comprises an immunotherapy. In some embodiments, the immunotherapy is immune checkpoint blockage. In some embodiments, the immune checkpoint is the PD-1/PD-L1 checkpoint. In some embodiments, the anti-PD-1/PD-L1 immunotherapy is selected from Pembrolizumab, Nivolumab, Durvalumab and Atezolizumab. Examples of therapeutic agents tailored to each subtype can be found in Rudin, et al., “Molecular subtypes of small cell lung cancer: a synthesis of human and mouse model data.”, Nature Reviews Cancer 19, 2019, 289-297; Roper, et al., “Notch signaling and efficacy of PD-1/PD-L1 blockade in relapsed small cell lung cancer.” Nature Communications 12 (2021), doi: 10.1038/s41467-021-24164-y; Gay, et al., “Patterns of transcription factor programs and immune pathway activation define four major subtypes of SCLC with distinct therapeutic vulnerabilities” Cancer Cell 39, 2021, 346-360.e7; Thomas, et al., “Therapeutic targeting of ATR yields durable regressions in small cell lung cancers with high replication stress.”, Cancer Cell 39, 2021, 566-579.e7; and Lissa, et al., “Heterogeneity of neuroendocrine transcriptional states in metastatic small cell lung cancers and patient-derived models.” Nat. Commun. 13, 2023 the contents of which are hereby incorporated herein by reference in their entirety. Any such therapy can be administered as the therapeutic agent.

In some embodiments, the method further comprises administering at least one anti-AIH therapeutic agent. In some embodiments, the administering is to a subject determined to suffer from AIH. In some embodiments, the method further comprises continuing to administer the anti-AIH therapeutic agent to a subject determined to have residual disease. In some embodiments, the method further comprises continuing to administer the anti-AIH therapeutic agent to a subject with reduced AIH load. In some embodiments, the method further comprises administering an alternative anti-AIH agent to a subject determined to have residual disease. n some embodiments, the method further comprises administering an alternative anti-AIH agent to a subject determined to not have a reduction in disease.

In some embodiments, the anti-AIH therapeutic agent is an immunosuppressant. In some embodiments, the immunosuppressant is a steroid. In some embodiments, the steroid is a corticosteroid. In some embodiments, the steroid is prednisone. In some embodiments, the immunosuppressant is azathioprine.

By another aspect, there is provided a computer program product comprising a non-transitory computer-readable storage medium having program code embodied thereon, the program code executable by at least one hardware processor to perform a method of the invention.

By another aspect, there is provided a system for performing a method of the invention.

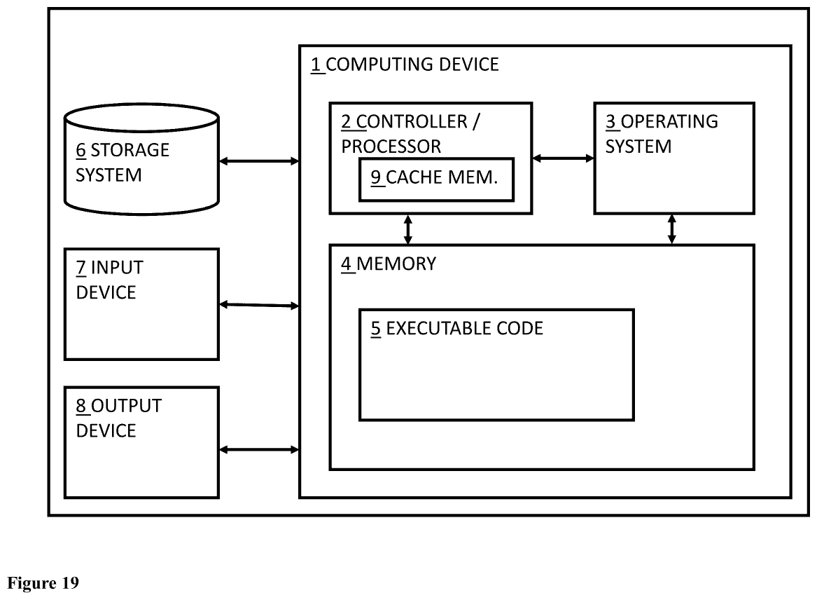

Reference is now made to FIG. 19, which is a block diagram depicting a computing device, which may be included within an embodiment of a system for performing a method of the invention, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task or script. Executable code 5 may be executed by processor or controller 2 possibly under control of operating system 3. For example, executable code 5 may be an application that may perform sequencing of the cfDNA, align sequencing reads to the human genome, calculate total reads, compare reads from a subject to those from a population or any or all of the above as further described herein. Although, for the sake of clarity, a single item of executable code 5 is shown in FIG. 19, a system according to some embodiments of the invention may include a plurality of executable code segments similar to executable code 5 that may be loaded into memory 4 and cause processor 2 to carry out methods described herein.

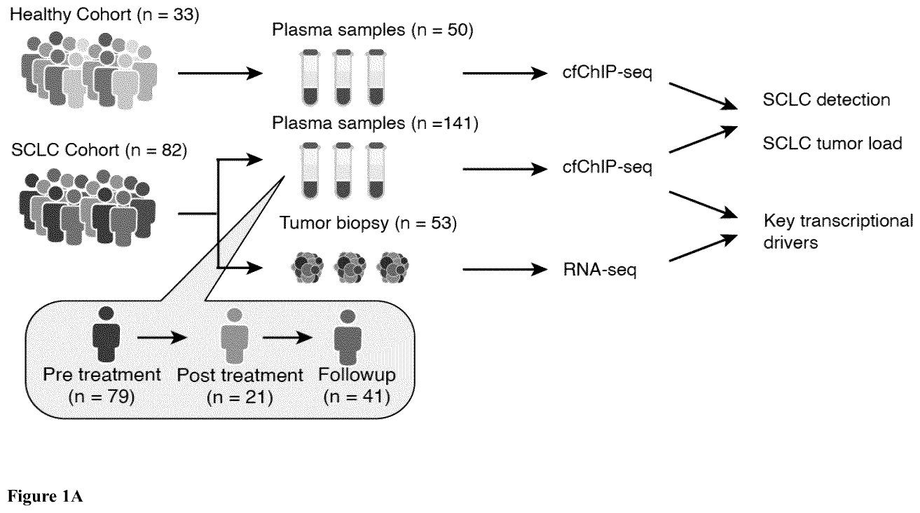

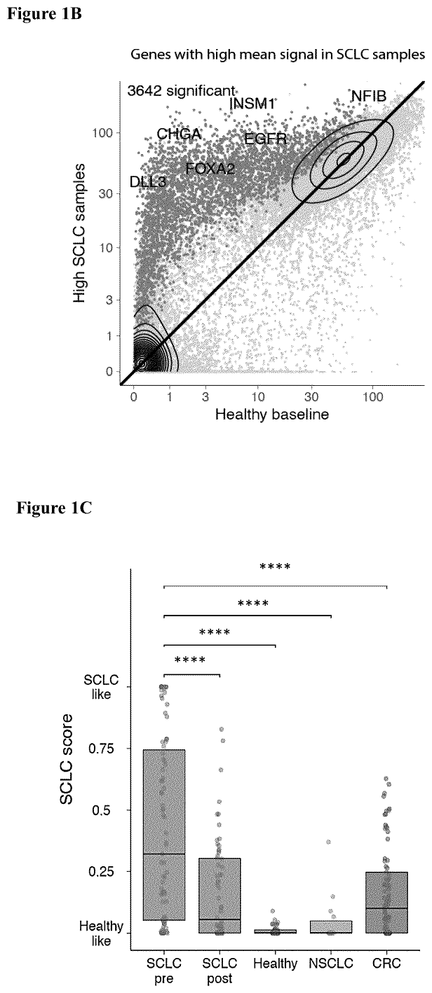

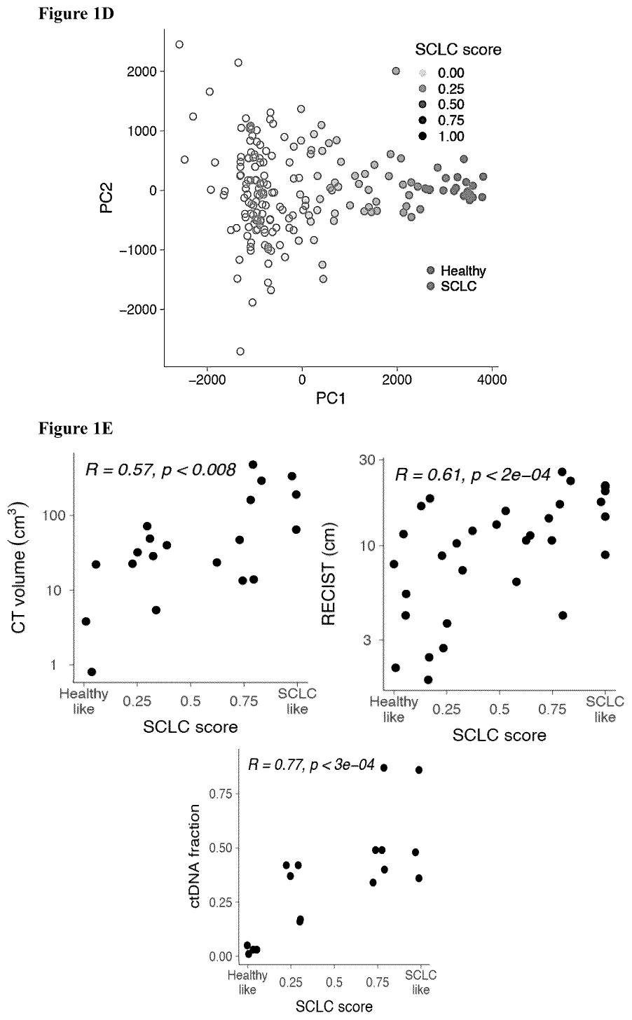

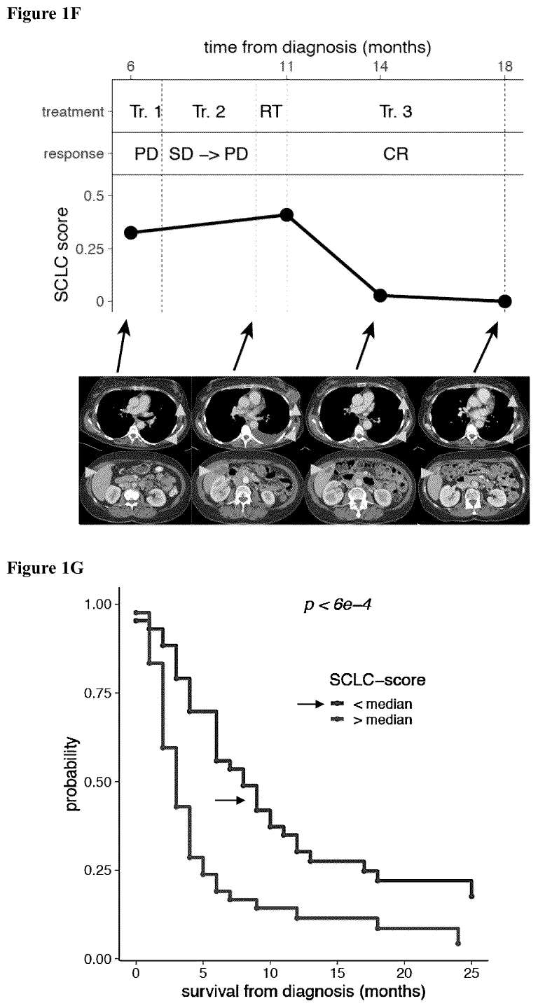

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Sequencing reads data may be stored in storage system 6 and may be loaded from storage system 6 into memory 4 where it may be processed by processor or controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units. In some embodiments, the system includes a device for sequencing.

By device for sequencing it is meant a combination of components that allows the sequence of a piece of DNA to be determined. In some embodiments, the device allows for the high-throughput sequencing of DNA. In some embodiments, the device allows for massively parallel sequencing of DNA. The components may include any of those described above with respect to the methods for sequencing. In certain embodiments the system further comprises a display for the output from the processor.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

Patients: We undertook an observational study using plasma collected from patients with small cell cancer who received care at the National Cancer Institute (NCI). Patients were enrolled in therapeutic clinical trials (ClinicalTrials.gov #NCT02484404; NCI protocol #15-C-0145; Clinical Trials.gov #NCT02487095; NCI protocol #15-C-0150; Clinical Trials.gov #NCT02769962; NCI protocol #16-C-0107; ClinicalTrials.gov #NCT03554473; NCI protocol #18-C-0110; and ClinicalTrials.gov #NCT03896503; NCI protocol #20-C-0009). We also collected samples from small cell cancer patients who were enrolled in the NCI thoracic malignancies natural history protocol (ClinicalTrials.gov #NCT02146170; NCI protocol #14-C-0105). If tumor samples were available, we also sequenced their RNA at matched or different time points of when the plasma was collected. Human subjects committee at NCI approved the studies; all patients provided written informed consent for plasma, tumor, and matched normal sample sequencing.

Tumor RNA sequencing: Formalin-Fixed, Paraffin-Embedded (FFPE) tumor tissue samples or frozen tumor samples in selected samples were prepared for RNA-seq. RNA enrichment was performed using TruSeq RNA Exome Library Prep according to manufacturer's instructions (Illumina, San Diego). Paired-end sequencing (2×75 bp) was performed on an Illumina NextSeq500 instrument. RNA was extracted from FFPE tumor cores using RNeasy FFPE kits according to the manufacturer's protocol (QIAGEN, Germantown, MD). RNA-seq libraries were generated using TruSeq RNA Access Library Prep Kits (TruSeq RNA Exome kits; Illumina) and sequenced on NextSeq500 sequencers using 75 bp paired-end sequencing method (Illumina, San Diego, CA). For transcriptomic analyses, raw RNA-Seq count data were normalized for inter-gene/sample comparison using TMM-FPKM, followed by log 2 (x+1) transformation, as implemented in the edgeR R/Bioconductor package.

Tumor volumetric estimation cfDNA Tfx: The DSP Circulating DNA kit from Qiagen was utilized to extract cell-free DNA from aliquots of plasma which were eluted into 40-80 uL of re-suspension buffer using the Qiagen Circulating DNA kit on the QIAsymphony liquid handling system. Library preparation utilized the Kapa Hyper Prep kit with custom adapters (IDT and Broad Institute). Samples were sequenced to meet a goal of 0.1× mean coverage utilizing the Laboratory Picard bioinformatics pipeline, and Illumina instruments were used for all of cfDNA sequencing with 150 bp and paired-end sequencing. Library construction was performed as known in the art. Kapa HyperPrep reagents in 96-reaction kit format were used for end repair/A-tailing, adapter ligation, and library enrichment polymerase chain reaction (PCR). After library construction, hybridization and capture were performed using the relevant components of Illumina's Nextera Exome Kit and following the manufacturer's suggested protocol. Cluster amplification of DNA libraries was performed according to the manufacturer's protocol (Illumina) using exclusion amplification chemistry and flowcells. Flowcells were sequenced utilizing Sequencing-by-Synthesis chemistry. Each pool of whole genome libraries was sequenced on paired 76 cycle runs with two 8 cycle index reads across the number of lanes needed to meet coverage for all libraries in the pool. Somatic copy number calls were identified using CNVkit (version 0.9.9) with default parameters. Tumor purity and ploidy were estimated by sclust and sequenza. cfDNA Tfx was estimated based on the somatic copy number alteration profiles using ichorCNA.

CTCs: CTCs were detected from 10 mL of peripheral blood drawn into EDTA tubes. Epithelial cell adhesion molecule (EpCAM)-positive CTCs were isolated using magnetic pre-enrichment and quantified using multiparameter flow cytometry. CTCs were identified as viable, nucleated, EpCAM+ cells that did not express the common leukocyte antigen CD45.

Radiological volumetric segmentation: We performed volumetric segmentation, a three-dimensional assessment of computed tomography as previously described (62). that may more accurately predict clinical outcomes than conventional evaluation by Response Evaluation Criteria in Solid Tumors (RECIST). Briefly, experienced radiologists reviewed the computed tomography sequences to determine the best ones to use for segmentation using the lesion management application within PACS (Vue PACS v 12.0, Carestream Health, Rochester, NY). We also assessed sizes of lesions by RECIST v.1.1.

Plasma cfChIP-seq

Sample collection: Plasma for cfDNA from EDTA. Sample should be processed within 2 hours. Centrifuge at 4 C at 1500×g for 10 minutes. Transfer plasma equally into Eppendorf tubes being careful not to disturb the leukocyte layer. Centrifuge a second time at 4 C at 10,000×g for 10 minutes. Without disturbing pellet, transfer plasma into standard cryovials. Barcode as plasma and enter sample note: ctDNA. Store at −80 C

Bead preparation: 50 μg of antibody was conjugated to 5 mg of epoxy M270 Dynabeads (Invitrogen) according to manufacturer instructions. The antibody-beads complexes were kept at 4° C. in PBS, 0.02% azide solution.

Immunoprecipitation, NGS library preparation, sequencing: Immunoprecipitation, library preparation and sequencing were performed by Senseera LTD., with certain modifications that increase capture and signal to background ratio. Briefly, ChIP antibodies were covalently immobilized to paramagnetic beads and incubated with plasma. Barcoded sequencing adaptors were ligated to chromatin fragments on the beads and DNA was isolated and next-generation sequenced.

Sequencing Analysis: Reads were aligned to the human genome (hg19) using bowtie2 (2.3.4.3) with ‘no-mixed’ and ‘no-discordant’ flags. We discarded fragments reads with low alignment scores (−q 2) and duplicate fragments.

Preprocessing of sequencing data was performed as known in the art. Briefly, the human genome was segmented into windows representing TSS, flanking TSS, and background (rest of the windows). The fragments covering each of these regions were quantified and used for further analysis. Non-specific fragments were estimated per sample and extracted resulting in the specific signal in every window. Counts were normalized and scaled to 1 million reads in healthy reference accounting for sequencing depth differences.

### Statistical Analysis

SCLC score: To estimate SCLC-score reflecting tumor-related fraction in samples, we performed a leave-one-out non-negative least square using the ‘nnls’ R package (1.4).

Denoting by G the number of gene promoter, and by S the number of samples in the reference cohort, a matrix X_{GXS+1} is composed containing the counts per promoters in the S healthy samples, with the addition of a ‘SCLC prototype’ composed on the mean promoter counts of the 10% SCLC samples with most genes significantly higher than healthy (n=15). For every vector Y_{1xG} of counts per promoter in the SCLC samples, we estimate the non-negative coefficient β by computing argmaxβ ∥Xβ−Y∥² subject to β≥0. A similar process is computed for healthy samples with the exception that for the iₜₕ sample we use the matrix X₋ᵢ where the column containing that sample is eliminated. The estimated β is normalized to 1 by computing

$$β = \frac{β}{{\sum }_{i} {β}_{i}} .$$

The tumor-related fraction is defined as the value of β_{SCLC}—the fraction assigned to the SCLC prototype. The healthy fraction of the iₜₕ sample is defined as Σ_{i≠SCLC} βᵢ, the sum of fractions assigned to all other healthy components.

An alternative approach was tested using a reference atlas of roadmap tissue data. In this approach the healthy fraction is set to be the sum of fractions assigned to common tissues observed typically in healthy samples (neutrophils, monocytes, megakaryocytes) and the tumor-related fraction is defined as 1−the tumor related fraction. The two approaches resulted in very similar estimations for the vast majority of samples (not shown).

We compute the principle component analysis (PCA) of all gene counts in all SCLC and healthy samples as implemented in the R ‘prcomp’ function. Scree plot was generated using the ‘fviz_eig’ function of the R ‘factoextra’ package (1.0.7).

Tissue signatures: Genomic regions selected for tissue specific markers are as known in the art. cfDNA of SCLC patients consists of tumor related cfDNA above the hematopoietic derived cfDNA observed in healthy individuals. Estimation of the absolute contribution of tissues to the cfDNA pool was calculated by multiplying the normalized signal per signature and the estimated number of reads in the sequencing library size which approximates the cfDNA concentration.

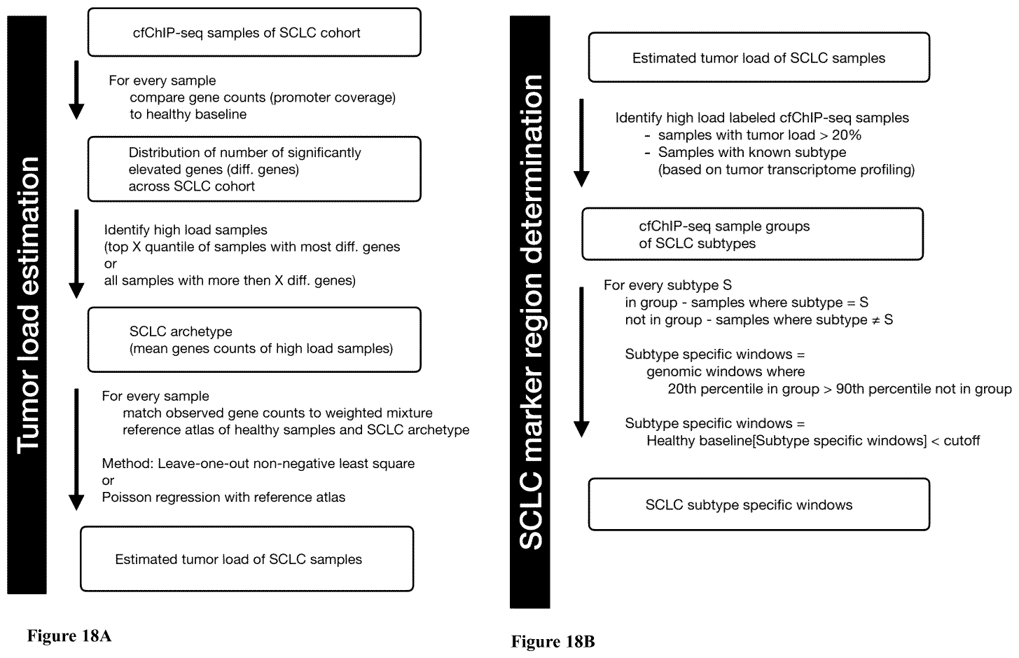

Lung single cell signatures: For the purpose of defining pulmonary cell-type specific signatures, we made use of published pulmonary scRNA-seq data (see, Travaglini, et al., “A molecular cell atlas of the human lung from single-cell RNA sequencing”, Nature, 587, 619-625, 2020, herein incorporated by reference in its entirety). In this publication, the authors defined cluster-specific marker gene sets, i.e., genes that are enriched for a specific cluster. The method of marker region identification is outlined in a flowchart in FIG. 18B. To increase the specificity of cell-type signature in the cfDNA context, we include only genes that meet the following criteria:
The percent of cells outside the cluster that express the genes is less than 0.1;
The average log fold-change expression in the cluster compared to other clusters is more then 2;
The adjusted p-value of the gene is less than 0.1;
The gene promoter read counts in cfDNA of healthy samples is less than 3.
   The last criteria is aimed to remove from the signature genes that are not lung-specific, rather, which appear in circulation as part of normal cell turnover. The genes included in every lung cell-type signature were selected from those known in the literature.

ChIP-RNA correlation: For examination of the tumor RNA-seq of tumor and plasma ChIP-seq relationship, we computed the Pearson correlation of all genes across the samples with matching tumor and plasma samples. Correlation was computed between 1+TSS counts of the ChIP-seq and log 2 (1+FPKM) and compared to the correlation achieved from a random permutation of the ChIP-seq samples. The correlation was computed for all samples with matching tumor-plasma (n=54) and separately for samples with SCLC-score >0.7 (n=19) and matching tumor.

Dynamic range of ChIP-seq gene g was calculated by the 95th percentile-5th percentile of the log 2 (1+normalized reads) across all plasma samples. High dynamic range genes were set to be genes with a dynamic range >2.

The dynamic range of the RNA-seq of gene g was calculated similarly for log 2 (1+FPKM). High dynamic range genes were defined as genes with a 3 fold-change between high and low samples.

Subtyping of plasma samples: Plasma samples with matching tumors were defined as TF-positive for the four TFs: ASCL1, NEUROD1, POU2F3 and YAP1 if the log 2 (1+FPKM) expression of the TF was above a threshold of 3 in the matching tumor. Samples defined as positive for more than one of the TF were not assigned a subtype, with the exception of samples positive for both ASCL1 and NEUROD1 (n=9) which were classified as ASCL1-NEUROD1. Samples that were not positive for any of the TF were not assigned a subtype.

Subtype specific genomic regions and subtype classifier: Tiling of the genome to genomic regions (windows) was performed as is known in the art (see Sadeh, et al., “ChIP-seq of plasma cell-free nucleosomes identifies gene expression programs of the cells of origin”, Nat. Biotech., 39, 586-598, 2021, herein incorporated by reference in its entirety). For every subtype, a signature of differential regions was defined as windows where the signal in the 20th percentile of samples within the group was greater than the signal in the 90th percentile of the samples outside the group. To increase specificity of the signatures, only samples with SCLC-score greater than 0.2 were used and genomics regions with mean expression greater than 0.3 among the healthy cohort were discarded. This process resulted in 45, 39 and 75 specific regions for the ASCL1, NEUROD1 and POU2F3 subtypes respectively. The full lists of genomic regions can be found in Tables 1-3.

For all plasma samples with SCLC-score greater than 0.05, the aggregated signal over the three signatures was computed and normalized by the SCLC-score to obtain the signature score. A manually curated threshold was set for the different signatures. The segregation achieved by the POU2F3 signature was the most pronounce and therefore a two-step classifier was designed whereupon in the first step samples are tested against that POU2F3 signature and in the second step, samples with a signature below the cutoff are tested against the other two signatures.

Patients (AIH cohort): Plasma samples of patients and healthy controls were collected in the pediatric Gastroenterology institute at Shaare Zedek Medical Center (SZMC). The samples were taken from patients under various clinical conditions: (1) patients undergoing liver biopsy for AIH diagnosis or other liver disease due to persistent elevation of liver enzymes or (2) to establish histological remission in patients with established AIH under treatment or (3) patients with established AIH under treatment with no adjacent liver biopsy. The control group comprises patients with normal liver biopsy or with no elevation of liver enzymes nor other liver disease. The study was approved by the Ethics Committees of the SZMC of Jerusalem (0269-19-SZMC). Informed consent was obtained from all individuals or their legal guardians before blood sampling.

Plasma cfChIP-seq (AIH)

Immunoprecipitation, NGS library preparation, and sequencing: Sample collection and handling, Immunoprecipitation, library preparation and sequencing were performed by Senseera LTD. as previously reported [ref], with certain modifications that increase capture and signal to background ratio. Briefly, ChIP antibodies were covalently immobilized to paramagnetic beads and incubated with plasma. Barcoded sequencing adaptors were ligated to chromatin fragments and DNA was isolated and next-generation sequenced.

Sequencing Analysis: Reads were aligned to the human genome (hg19) using bowtie2 with ‘no-mixed’ and ‘no-discordant’ flags. We discarded fragments reads with low alignment scores (−q 2) and duplicate fragments.

Preprocessing of sequencing data was performed as previously described. Briefly, the human genome was segmented into windows representing TSS, flanking to TSS, and background (rest of the windows). The fragments covering each of these regions were quantified and used for further analysis. Non-specific fragments were estimated per sample and extracted resulting in the specific signal in every window. Counts were normalized and scaled to 1 million reads in healthy reference accounting for sequencing depth differences.

### Statistical Analysis

Differential genes compared to healthy: Statistical analysis of differential genes was performed as previously reported. Briefly, for every gene in every sample we test whether the observed gene coverage is higher than expected according to the healthy mean/variance estimated from a control group of XX self-reported healthy donors. Using the background rate of every sample and the scaling factor accounting for the sequencing depth, we define an expected distribution and estimate the probability of the observed coverage under the null hypothesis that the sample came from the healthy population. Genes with a FDR corrected P-value below 0.001 are reported as significantly elevated in the sample. We are aware of the fact that the healthy cohort used to define the baseline consists of healthy adults. However, testing a separate cohort of samples from healthy children, we observe virtually no difference which justifies the use of this reference.

Cell type composition of samples (deconvolution); To estimate the tissue composition of every sample, we used a non-negative least square model as implemented in the ‘nnls’ R package (1.4). Given reference matrix X\hat{ }(εK×G) of the genes in K cell types and vector [symbol] Y\hat{ }(εG) [symbol] of observed gene counts in a sample, the objective is identifying non-negative coefficients β\hat{ }(cell-type proportion) by solving [symbol] argmin [symbol] _B∥ [symbol] X\hat{ }Tβ [symbol] _-Y∥_2\hat{ }2 subject to β_i>0 and Σ_i β_i=1. For reference tissue atlas we used 182 samples from the Roadmap and Blueprint H3K4me3 ChIP-seq data. Estimated coefficients of similar cell-types were summed and the final composition across 36 distinct cell-types is shown. These results were reproducible when using different features for the regression and with other regression models.

Liver single cell signatures: Identification of liver specific cell-types genes was done based on liver specific marker genes from published liver scRNA-seq data. To increase the specificity of cell-type signature in the cfDNA context, we exclude genes with mean counts above 1 in the healthy reference assuming their promoter is marked by H3K4me3 in non-liver cells contributing to the circulation. This filtering step resulted in a reduced number of marker genes particularly in the liver immune cells.

Expected, residual and unexplained gene counts: For every sample we define the expected gene counts to be the mean gene counts of the composing cell types weighted by the contribution fraction of the cell-type as described above (X·β\hat{ }). To overcome misleading results due to missing tissues in the reference atlas, we added to the atlas an additional healthy profile derived from a large cohort of healthy cfChIP-seq samples.

The residual is defined as [symbol] log [symbol] _2 (1+observed)− [symbol] log [symbol] _2 (1+expected). To further account for inter-tissue variability, we estimate the expected variance of every gene based on the weighted empirical variance observed in the replicates of the tissues composing the samples and test whether the null hypothesis that the observed counts are negative binomial distributed with that mean and variance can be rejected.

Formally, given a set of genes G, let:
S_1, S_2 . . . . S_n—set of cfChIP-seq samples
Y_(i,g)—coverage of gene g in samples i
B_(i,g)—background reads in gene g of sample i
Q_i—normalization factor of sample i (sequencing rate)
k=1, . . . . K—set of reference cell-types
X_(k,g)—coverage of gene g in cell-type k
β\hat{ }i∈R_(≥0)\hat{ }k—estimated fractions of cell-types composing sample i
m_(k,g),σ_(k,g)—mean and standard deviation of gene g in cell-type k, were
μ_(k,g),m_(k,g) are estimated as known in the art.
   For every sample i the objective is to estimate the distribution:

p(Y_(i,g)|B_(i,g),Q_i,β\hat{ }_i,u_(k,g),σ_(kg)).

Due to discrete sampling in library preparation and sequencing we assume that Y_(i,g) is Poisson distributed depending on the expected counts and sequencing depth.

Y_(i,g)˜Poisson(1/Q_iη_(i,g)+B_(i,g)).

where η=X·β\hat{ }.

We approximate the distribution of [symbol] Y_(i,g) [symbol] as negative binomial NB(μ_(i,g),σ_(i,g)\hat{ }2).
Using linearity of expectation and the law of total variation we can match the mean and variance of the negative binomial to that of the exact distribution:

$$μ = 1 / Q E [ η ] + B\_ = 1 / Q \sum \_k β\_k m\_k + B$$

$$σ \hat{} 2 = μ + 1 / Q \hat{} 2 Var [ η ] = μ + 1 / Q \hat{} 2 \sum \_k β\_k \hat{} 2 σ\_k \hat{} 2$$

For every gene in every sample we compute the probability of p_NB (x≥Y_(i,g)|μ,σ\hat{ }2). Unexplained genes were defined as genes where the FDR-corrected q-value was less than 0.001 in at least 3 AIH samples.

### Example 1: SCLCs have Distinct cfChIP-Seq Signals that Track Tumor Burden and Prognosis

Plasma samples were collected, processed, and H3K4me3 ChIP-seq performed directly on ˜1 ml of plasma with a median of 3.1 million unique reads sequenced per sample. For every gene, the number of normalized reads mapping to its respective transcriptional start site (TSS) regions was computed, resulting in gene counts resembling transcription counts in RNA-seq data. Comparing the gene counts in SCLC plasma samples to healthy reference samples, we found significantly elevated counts in hundreds to thousands of genes (FIG. 1B, 6A). 3642 genes had significantly higher coverage (q<0.001) in at least 3 SCLC samples.

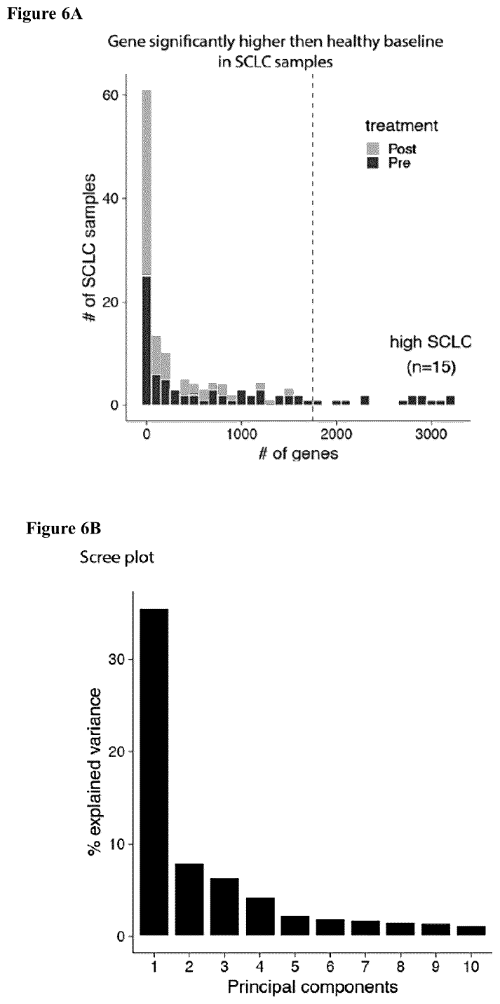

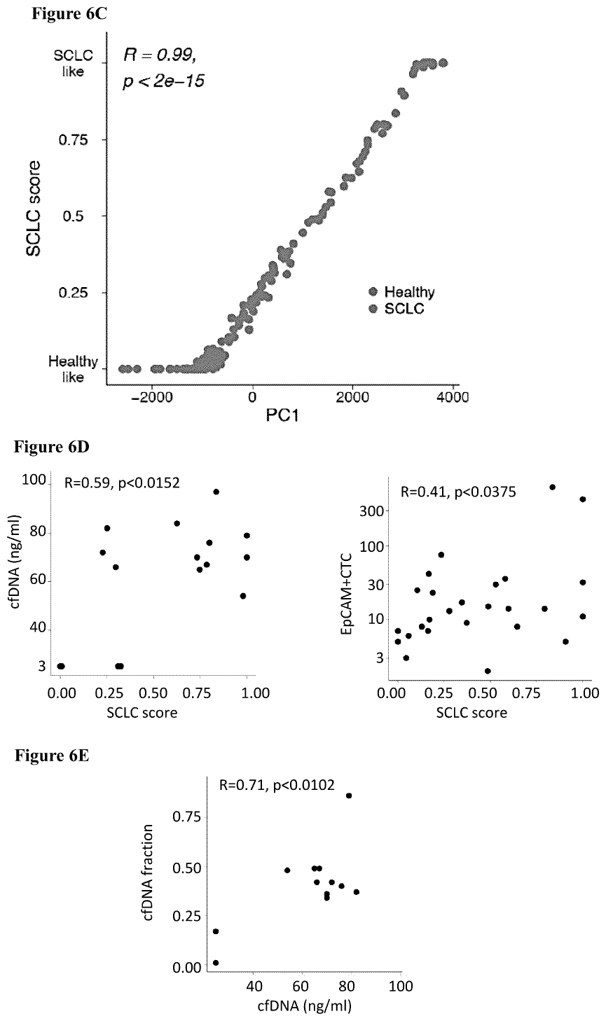

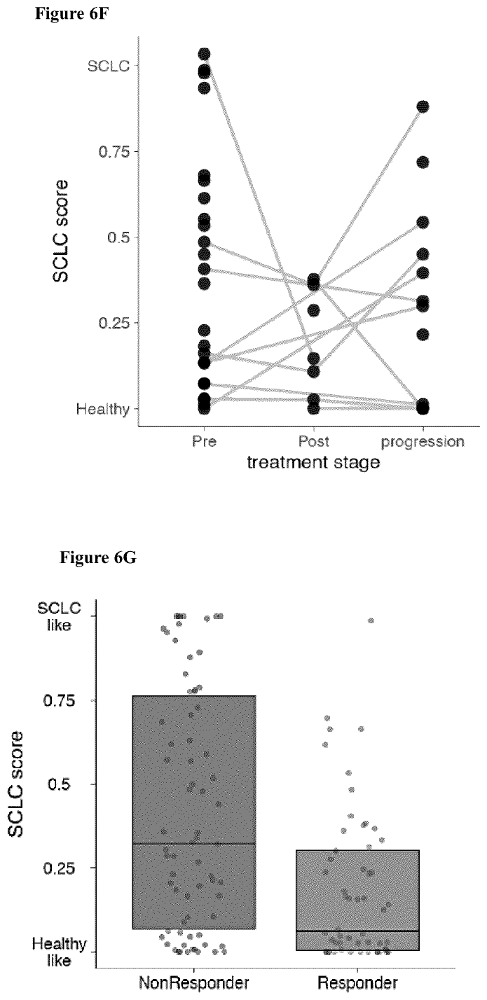

cfDNA of cancer patients consists of DNA fragments originating from tumor cells and DNA released by normal cells, commonly of the hematopoietic lineage. Tumor fractions can vary substantially, depending on a number of variables including tumor burden and growth activity. To account for this variability, we estimated an ‘SCLC-score’ reflecting the proportion of tumor-derived cfDNA. The score is based on a linear regression that matches the observed profile (gene counts) in a sample to a weighted mixture of reference cfChIP-seq profiles. Reference consisted of samples from healthy individuals and a SCLC archetype based on the 10% of SCLC samples (n=15) with the highest number of differential genes compared with healthy samples (FIG. 6A). The SCLC-score ranged from 0 (‘healthy like’) to 1 (‘SCLC like’), with a median of 0.32 and 0.05 in SCLC samples collected before and after treatment, respectively, indicating a decrease of SCLC tumor fraction by the therapeutic interventions. In contrast, plasma from healthy subjects and patients with non-SCLC cancers displayed absent or very low SCLC-scores (median of 0 in healthy and NSCLC, 0.1 in CRC; Anova p<10-15. FIG. 1C). To assess our estimation in an unsupervised manner, we performed principal component analysis (PCA) on the gene counts of healthy controls and SCLC samples (Methods). Superimposing the estimated SCLC-score over the 2-dimensional PCA plot, we found that PCA1—the axis contributing most to sample variability—correlated almost perfectly with estimated tumor load (r 0.99, p<10-15, FIG. 1D, 6B-6C). A flow chart of the method of determining tumor load is provided in FIG. 18A.

Importantly, cfChIP-seq SCLC-scores were significantly correlated with multiple other measures of tumor fraction including somatic copy number alteration-based estimates from ultra-low pass whole genome sequencing, circulating tumor cell (CTC) counts, total cfDNA concentrations (Pearson r 0.77, 0.43 and 0.62; p<3e-4, 0.01, and 0.03, respectively), computerized tomography scan-based volumetric tumor assessments, and standardized unidimensional tumor measurements (Pearson r and p: 0.57 and 0.61; <0.008 and <2e-4, respectively, FIG. 1E, 6D-6E). Furthermore, cfChIP-seq SCLC-scores tracked radiographic tumor burden through the treatment time course (FIG. 1F), and predicted treatment response and overall survival (FIG. 1G, 6F-6G).

Taken together, these results demonstrate the potential utility of cfChIP-seq to non-invasively assess cfDNA tumor fraction in patients with advanced SCLC.

### Example 2: CfChIP-Seq Recovers SCLC Tissue and Cellular Origins

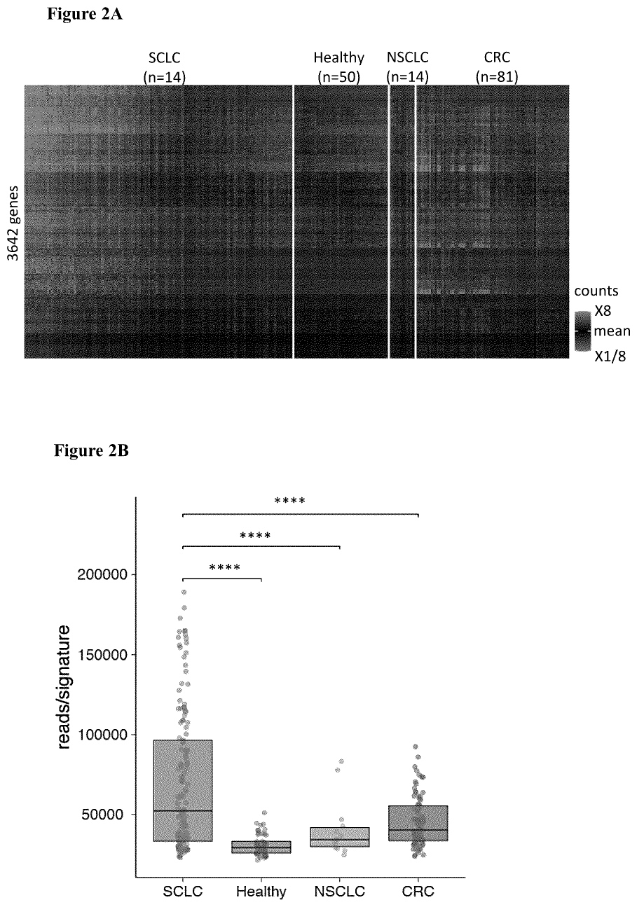

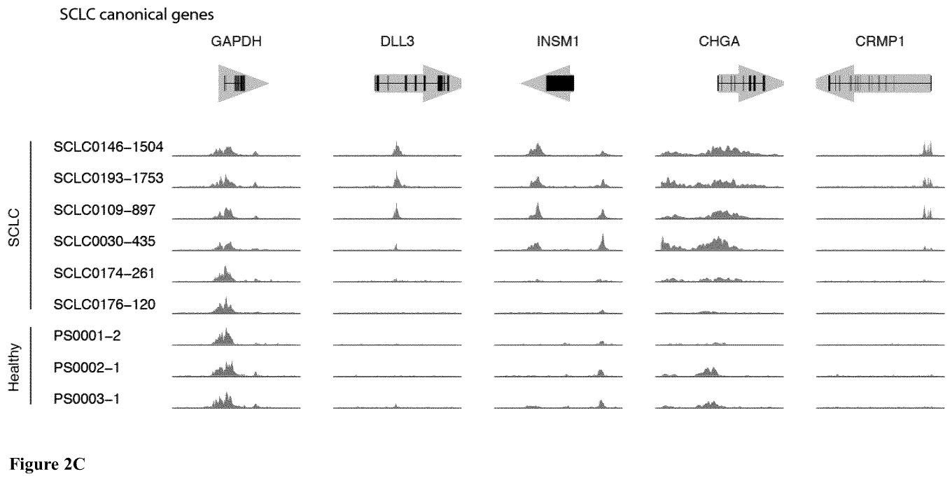

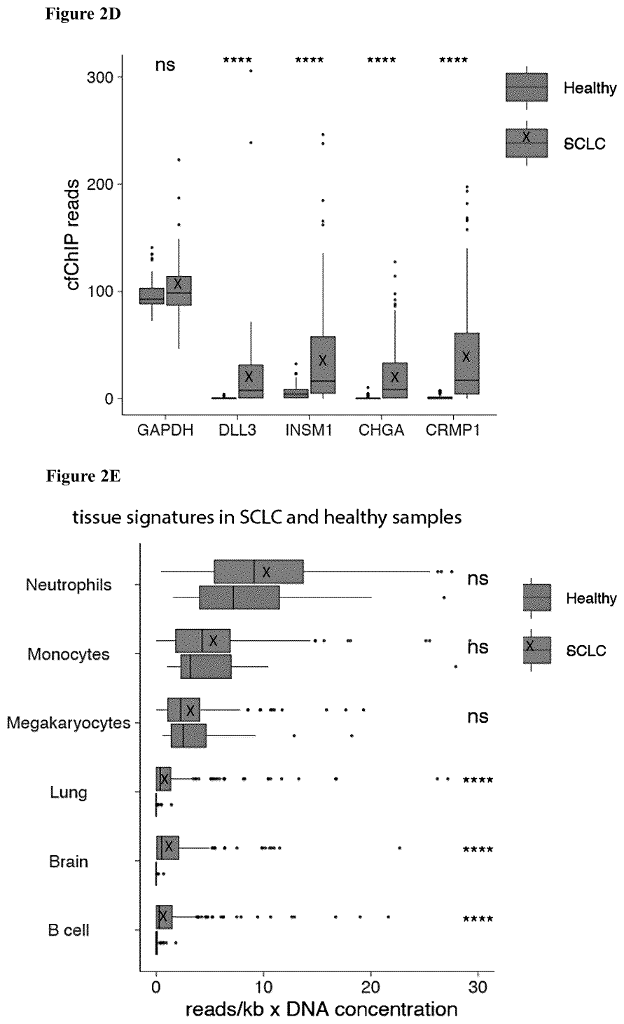

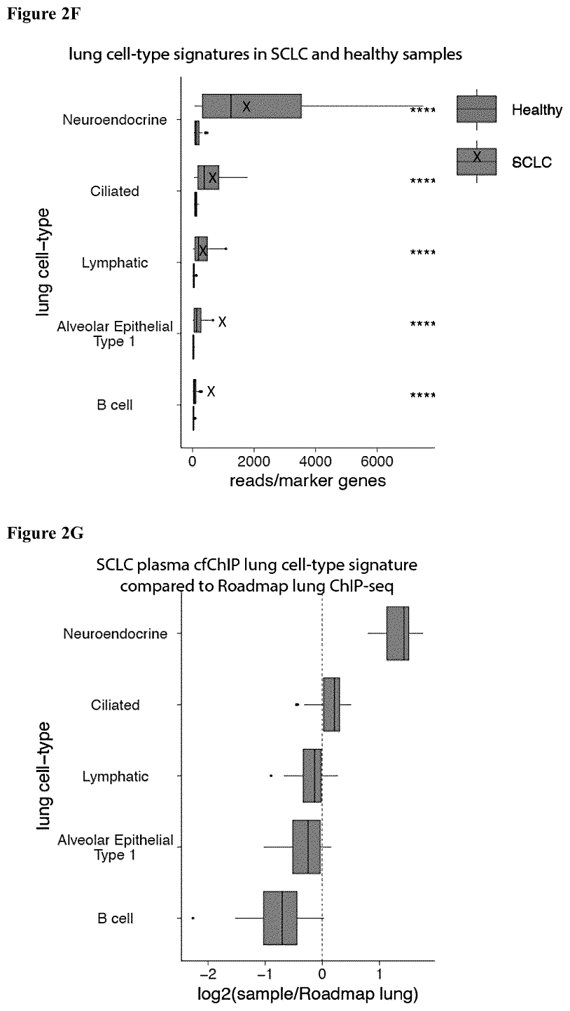

We next explored whether plasma cfChIP-seq signals might profile the epigenetic state of tissue and cell of origin of SCLC tumors. We found that many of the more than 3500 genes with significantly elevated coverage in SCLC plasma (FIG. 1B) were elevated specifically in the SCLC samples compared with healthy controls or other cancers (FIG. 2A-2B). SCLC-signature genes were highly enriched for genes expressed in SCLC cell lines and pulmonary neuroendocrine cells (gene overlap of 277/465 and 53/92; p<4.710-97 and <3.510-15 respectively). Specifically, SCLC plasma displayed high counts of canonical genes such as DLL3, INSM1, CHGA and CRMP1-significantly higher than in healthy samples (FIG. 2C-2D).

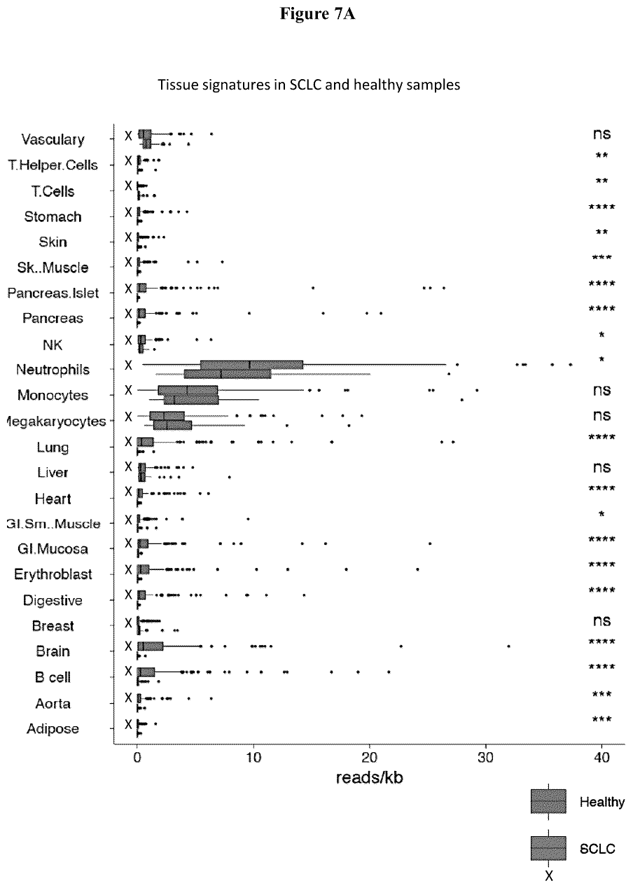

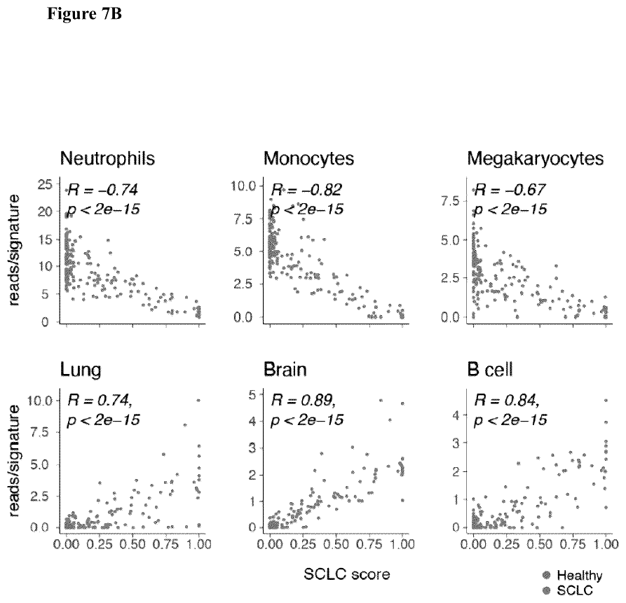

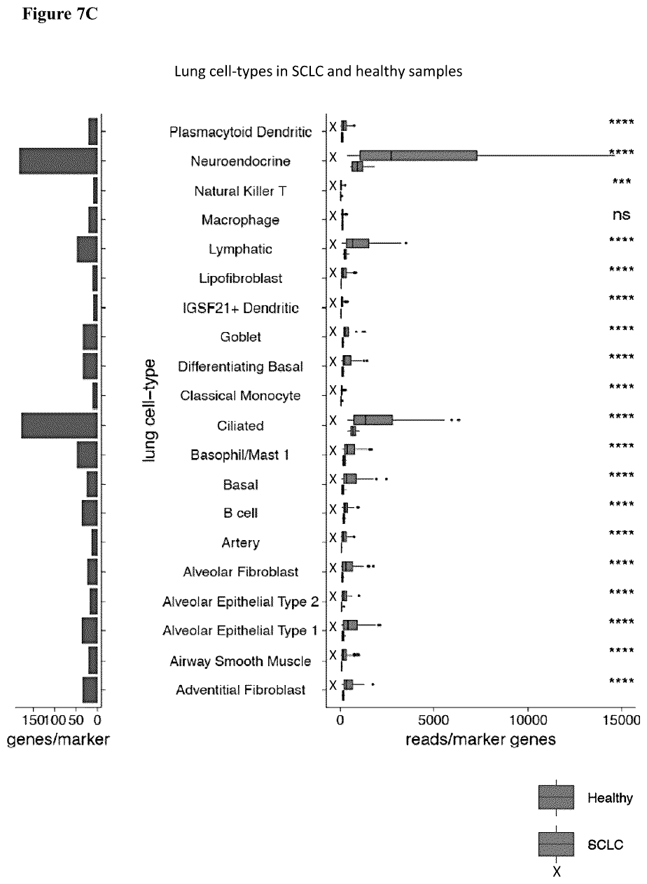

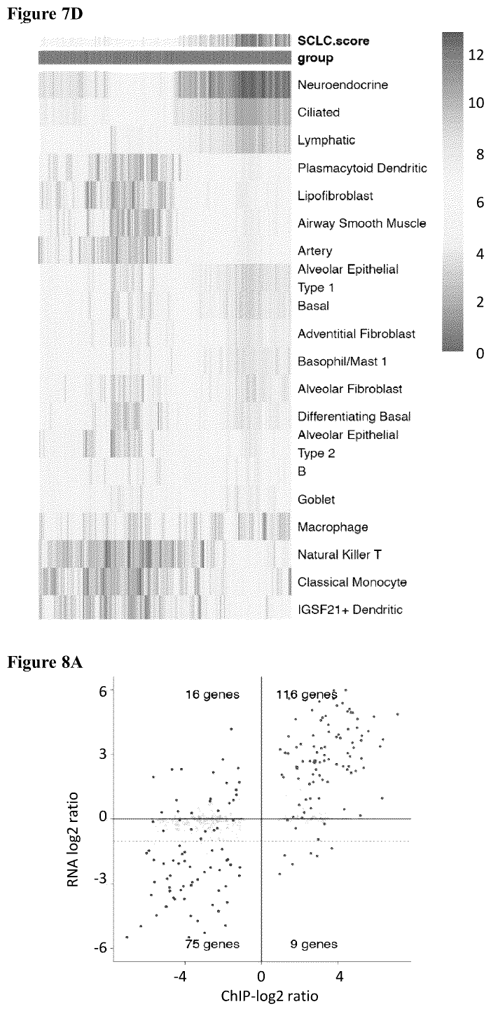

In order to characterize the tissue-specific contributions to cfDNA, we defined a set of tissue-specific genomic loci from ChIP-seq reference data (Methods). Using this approach, we verified that signals in healthy plasma are mainly derived from neutrophils, megakaryocytes, and monocytes. In contrast, signals in SCLC plasma were derived also from lung, brain, and B-cells, suggesting contributions from cells of pulmonary, neuronal, and lymphocyte lineages (FIG. 2E, 7A). As expected, ChIP-seq signatures for lung, brain, and B-cell were positively correlated with the SCLC-score (FIG. 7B).

To further test whether cfChIP-seq can provide clues to the cell-of-origin, we used pulmonary cell-type-specific signatures derived from lung single cell RNA-seq data. Across the ˜50 cell-types examined, spanning lung epithelial, endothelial, stromal, and immune cells, we found remarkable enrichment of the neuroendocrine cell type in SCLC plasma compared with healthy plasma (FIG. 2F). This result was especially striking since neuroendocrine cells constitute only 0.13% of healthy lung tissue. Neuroendocrine cell signature was also significantly elevated in SCLC plasma compared with normal lung tissue (FIG. 2G). Signals of other lung cells including ciliated cells and alveolar epithelial type 1 cells were also higher in SCLC plasma compared to healthy donor plasma (FIG. 2F, 7C-7D), hinting at the possibility of SCLCs arising additionally from non-neuroendocrine cells-of-origin as previously described in the literature or injury to the specific cells.

Together, these findings demonstrate that cfChIP-seq recovers the unique epigenetic states of tissue and cells-of-origin of SCLC tumors.

### Example 3: Plasma cfChIP-Seq Informs Tumor Gene Expression

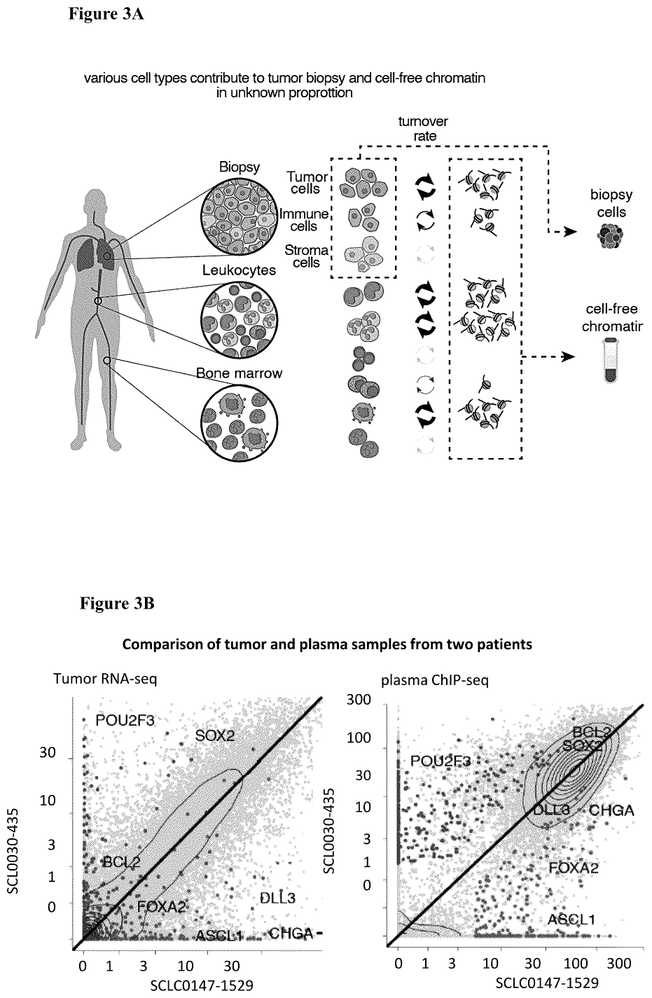

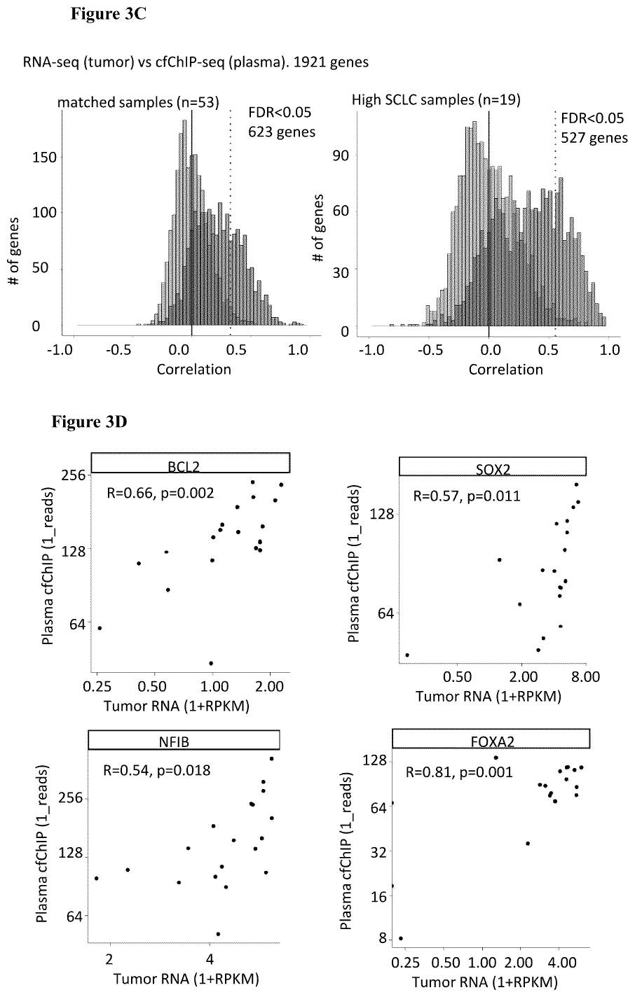

Recent studies reveal that SCLC tumors are transcriptionally heterogeneous (FIG. 3A). We sought to understand whether cfChIP-seq reflects gene expression patterns of SCLC tumors.

As an example, among the high SCLC-score plasma, one sample (SCLC030.435) in particular exhibited markedly higher signal at several genes (e.g., POU2F3 and BCL2) compared to the other high SCLC-score samples (FIG. 3B). Examining time-point matched tumor RNA-seq of these samples revealed differential expression of many of the same genes, indicating that differential cfChIP-seq signals might indeed reflect tumor gene expression (FIG. 3B, 8A).

To systematically examine the relationship between circulating chromatin state and tumor gene expression, we computed for each gene the correlation between plasma cfChIP-seq counts and tumor RNA-seq TMM-normalized FPKM values (n=53 samples with available paired plasma and tumor). Excluding genes with low dynamic range in cfChIP-seq or RNA-seq (Methods), a significant positive correlation was observed in more than 25% of the genes (623 genes with q<0.05; Pearson 0.33<r<0.93). cfDNA tumor fraction may confound this correlation, with high tumor fraction plasma samples expected to have a better correlation with tumor gene expression. Indeed, when comparing only the high SCLC-score samples with matched tumor (n=22), a similar number of significant genes was observed (527 genes with q<0.05, 416 of them overlap with the significant genes in the full dataset), but with a higher correlation (Pearson 0.55<r<0.98. FIG. 3C, 8B) despite the reduced statistical power. In particular, a high positive correlation was observed between cfChIP-seq and RNA-seq read counts for several important SCLC oncogenes such as BCL2, NFIB, SOX2 and FOXA2 (FIG. 3D).

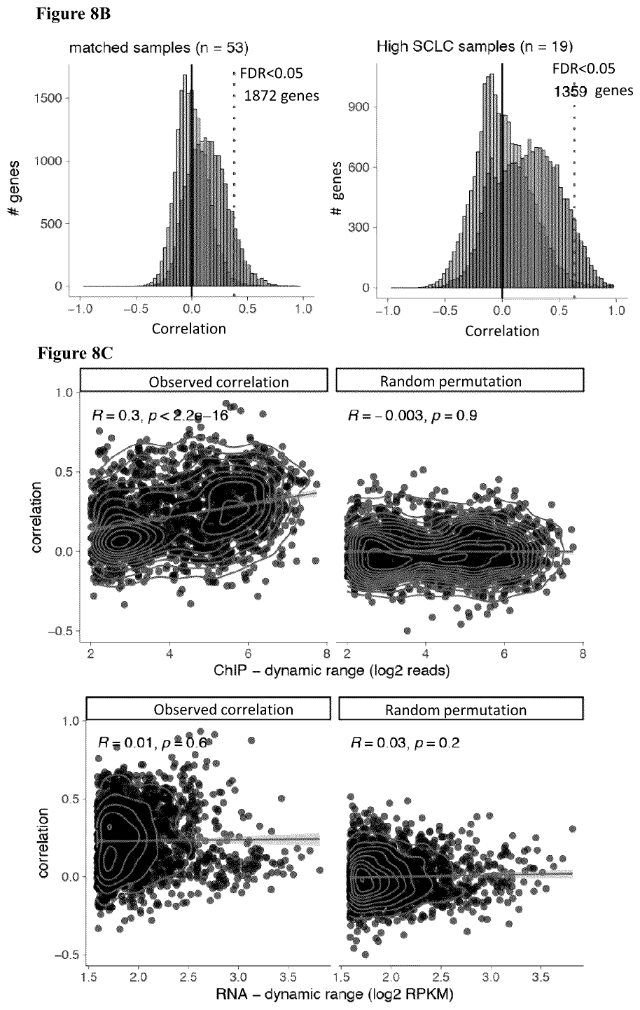

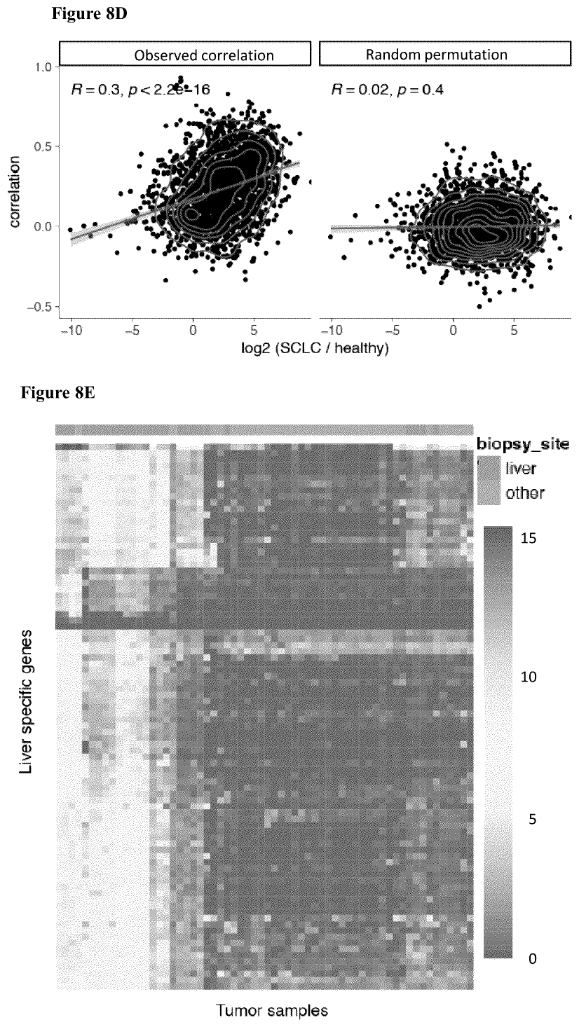

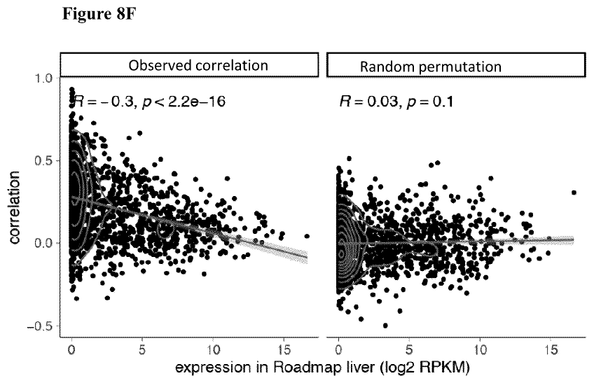

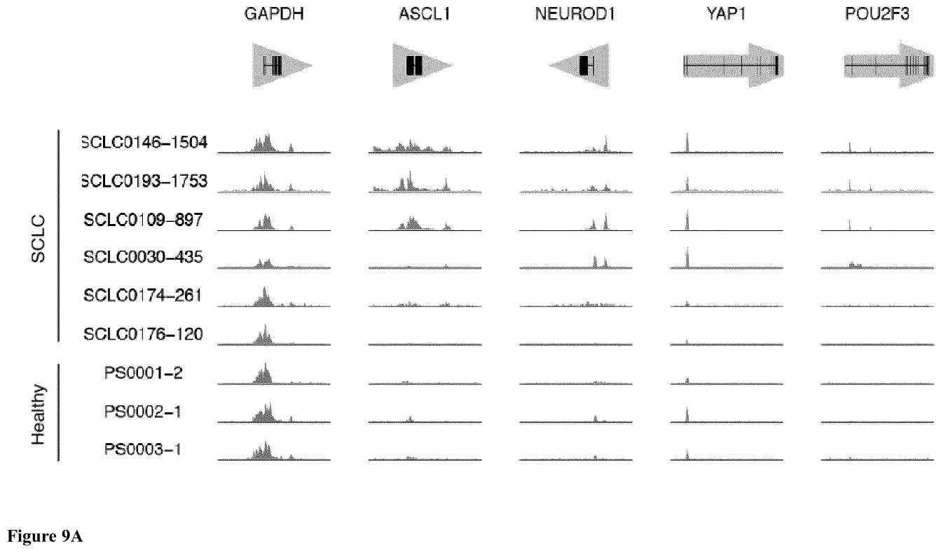

To better understand the concordance between tumor RNA-seq and cfChIP-seq, we examined reasons why some genes, and not others show a positive correlation. A basic difference between chromatin and transcription is that, at the single cell level, an active chromatin mark is essentially binary (promoter marked or not marked), while RNA levels have a large dynamic range. Indeed, genes with cfChIP-seq high dynamic range tend to have higher correlation (FIG. 8C). Another possible source of divergence between the cfChIP-seq and tumor RNA-seq is the interference of the non-tumorous tissues contributing to each measurement (FIG. 3A, 8D-8F).

Overall, these findings demonstrate that cfDNA chromatin state assessed by cfChIP-seq informs the tumor gene expression programs, especially in plasma samples with high tumor fraction, and reveal important variables that affect the concordance between cfDNA chromatin state and tumor gene expression.

### Example 4: Plasma cfChIP-Seq Predicts Tumor Gene Expression of SCLC Lineage-Defining Transcription Factors

Given the concordance between tumor gene expression and plasma chromatin state, we next focused on specific genes with known relevance to SCLC tumorigenesis.

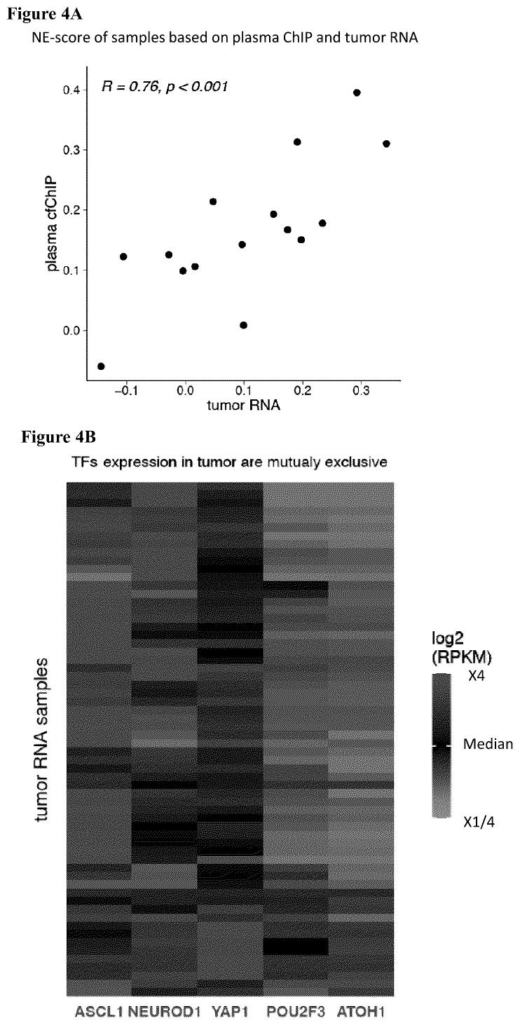

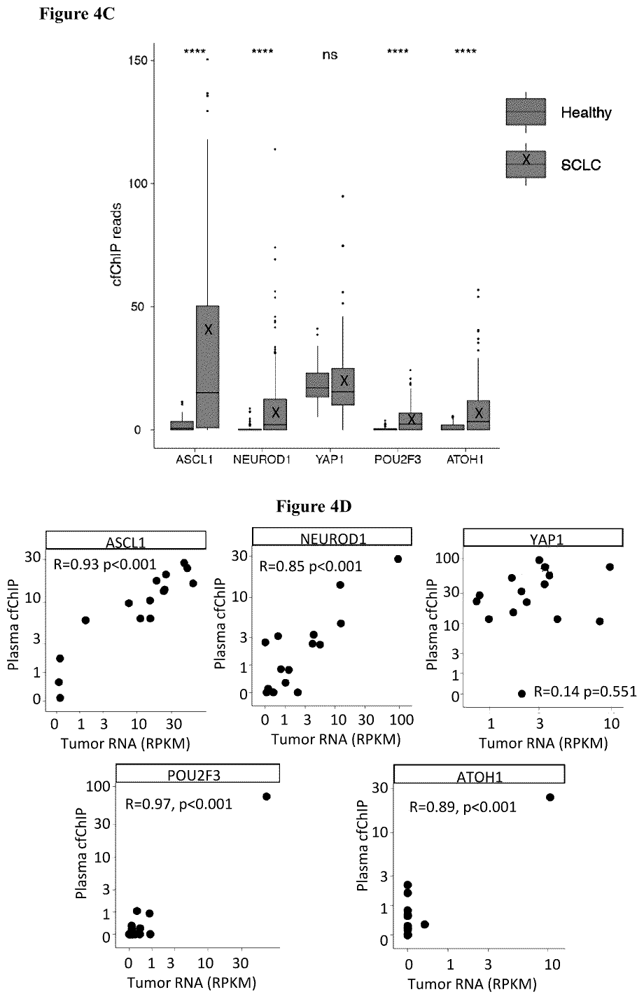

Transcriptional signatures of SCLC heterogeneity converge on two major cell states, namely neuroendocrine (NE) and non-neuroendocrine (non-NE). Using a previously validated signature (Zhang, et al., “Small cell lung cancer tumors and preclinical models display heterogeneity of neuroendocrine phenotypes”, Trans. Lung Cancer Res., 7, 32-49 2018, herein incorporated by reference), we estimated neuroendocrine gene expression scores in plasma and tumor, and found a significant positive correlation between scores derived from plasma cfChIP-seq and tumor RNA-seq (FIG. 4A; Pearson r 0.76 and p<0.001).

The SCLC neuroendocrine cell states are further characterized by expression of key lineage-defining transcription factors, ASCL1 and NEUROD1 defining NE cell states and POU2F3 defining non-NE cell states. A fourth subgroup has been characterized by expression of YAP1 or low expression of all three transcription factors accompanied by an inflamed gene signature. Most tumors in our cohort had high expression of NE-lineage defining genes ASCL1 and NEUROD1, with co-expression of both genes in some cases (FIG. 4B). Expression of POU2F3, YAP1, and a newly described subtype ATOH1 was seen in ˜7% of tumors and were largely mutually exclusive.

We sought to examine whether the expression of SCLC lineage-defining transcription factors in the tumor is reflected in the plasma cfChIP-seq. We found significantly elevated counts of ASCL1, NEUROD1, and POU2F3 in SCLC samples in contrast to barely detectable levels in healthy plasma. In contrast, YAP1 counts were similar among healthy and SCLC plasma samples (FIG. 3C, 9A), likely due to YAP1 activity in normal tissue contributing to cfDNA, as suggested by H3K4me3 of YAP1 promoter regions in normal tissue.

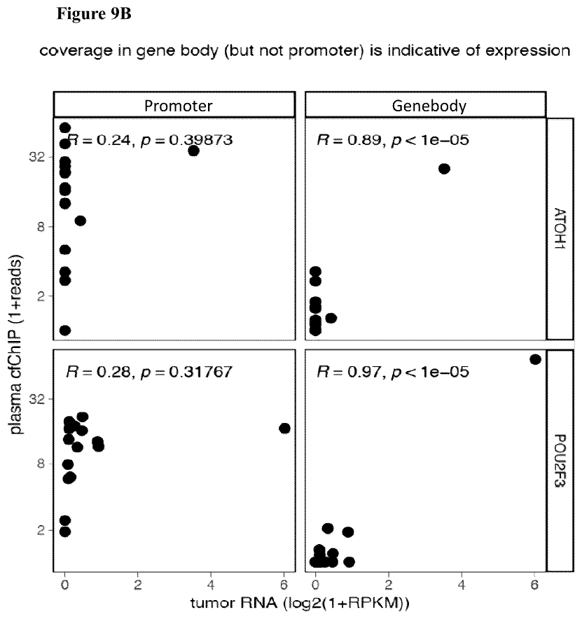

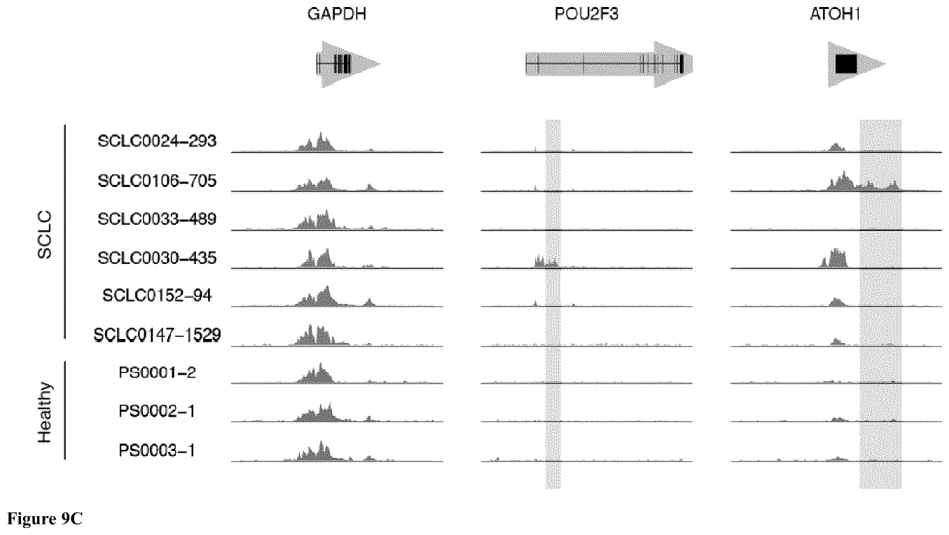

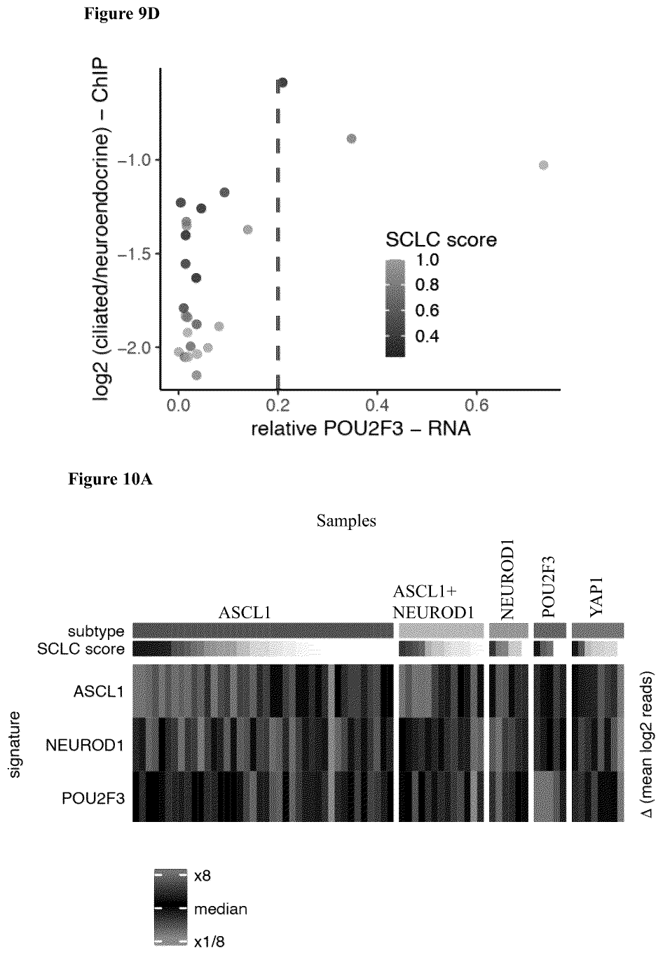

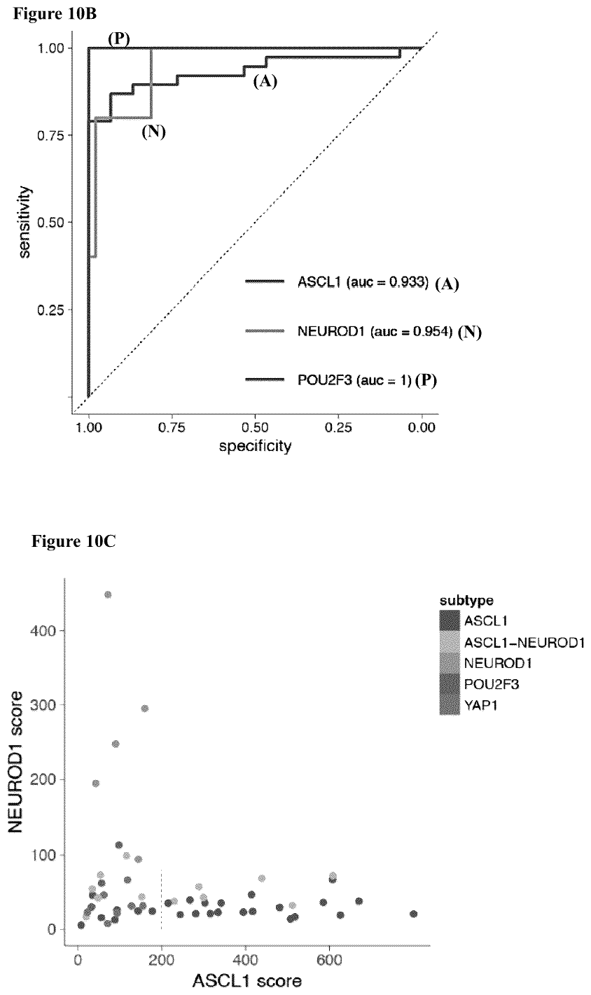

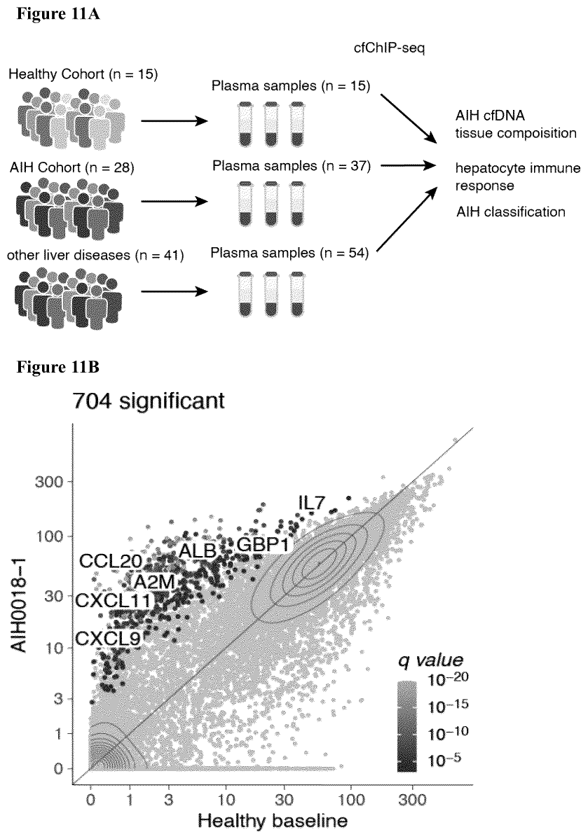

To evaluate whether these patterns are indicative of tumor gene expression in individual patients, we assessed the concordance between high SCLC-score plasma cfChIP-seq counts and gene expression levels derived from time-point matched tumor transcriptome (n=18). A strong correlation between cfChIP-seq and tumor RNA-seq was observed for three of the transcription factors (ASCL1, NEUROD1 and POU2F3) (Pearson r 0.93, 0.85, and 0.97; p<1*10-5, <7*10-5, and <1*10-5 respectively), which were also absent from healthy control cfDNA. A similarly high positive correlation was observed between plasma cfChIP-seq and tumor RNA-seq of ATOH1 (Pearson r 0.89; p<1*10-5; FIG. 4D). Notably, the TSS of POU2F3 and ATOH1 is marked by H3K4me3 in many of the SCLC samples, however, only in a small subset of them do the signals span beyond the TSS region to a wide region of approximately 10 KB suggesting that in these cases they are involved in cell-type specific functions. We find these additional regions correlate best with gene expression in the tumor (FIG. 9B-9C).

Revisiting the cell-type source of the plasma cfChIP-seq samples, we noticed that the three samples with high relative POU2F3 expression in the respective tumors, had high ciliated to neuroendocrine cell ratio (FIG. 9D). This is in accordance with their classification as non-NE tumors and the selective expression of POU2F3 in tuft cells, a rare chemosensory cell type in the pulmonary epithelium.

### Example 5: Multi-Gene Signatures Enable Subtyping of SCLC from Plasma cfChIP-Seq

Next, we sought to examine the possibility of subtyping SCLC tumors directly from plasma using cfChIP-seq. SCLC tumors are typically categorized by the expression of lineage-defining transcription factors, suggesting that the cfChIP counts of these genes would suffice for plasma-based categorization. However, despite the good agreement between the RNA-seq and cfChIP-seq, relying only on single genes for subtype discrimination is challenging, especially considering the low concentration of tumor-derived cfDNA in many samples. We hypothesized that a multigene signature for each subtype would be a more robust approach, increasing the predictive power and enabling subtyping of a wider range of plasma samples.

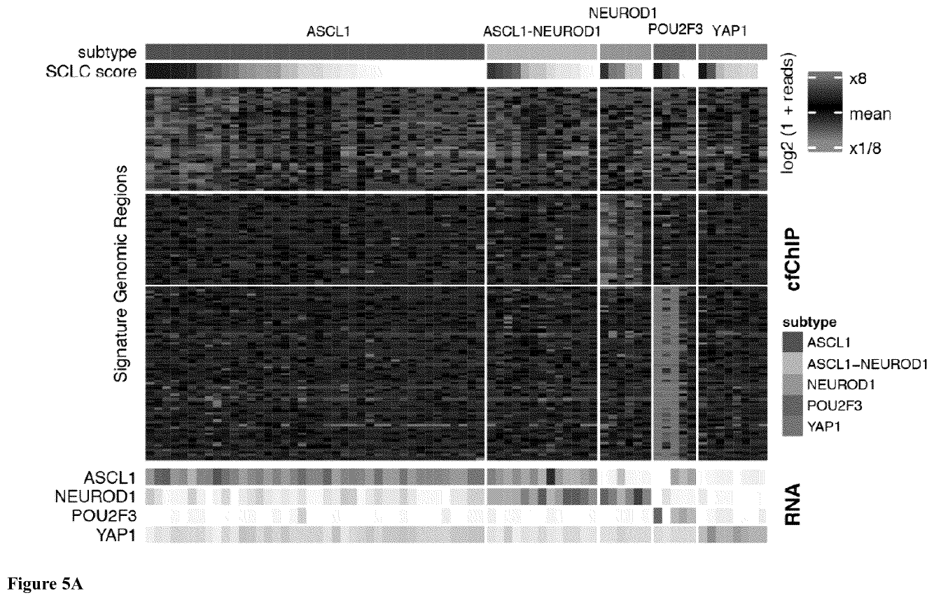

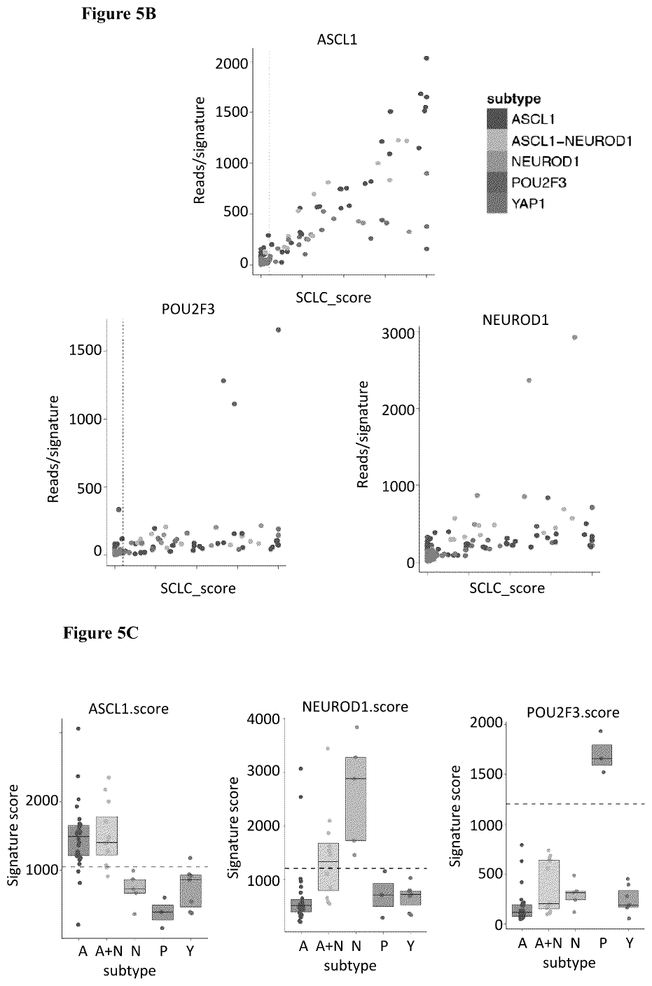

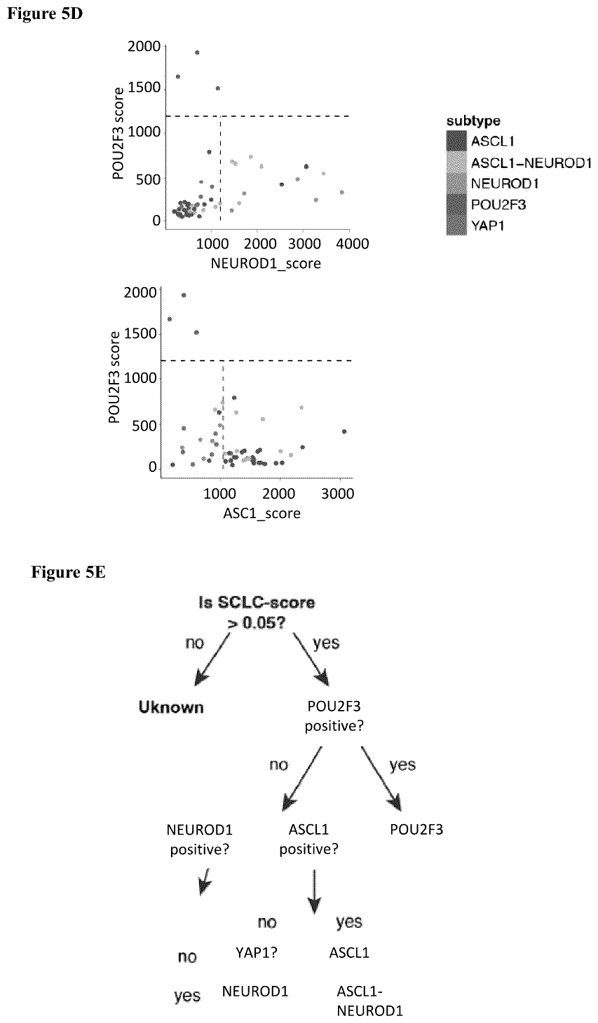

Since our cohort included only one sample with high expression of ATOH1, we focused on designing a classifier to distinguish between the other four subtypes (ACSL1, NEUROD1, POU2F3, and YAP1). Plasma samples with high SCLC-score were assigned a subtype by the matching tumor RNA-seq (Methods). We then searched for genomic regions with differential cfChIP-seq signals among the various groups (Methods). This led to the identification of several dozens of genomic regions that discriminate between the ASCL1, NEUROD1 and POU2F3 subtypes (FIG. 5A, 10A, Tables 1-3). We did not find regions that are uniquely high in YAP1 subtype. The aggregated read counts in these genomic regions displayed a linear relationship with the SCLC-score in corresponding samples but were constant and low in other samples, even those with high SCLC-scores (FIG. 5B). We normalized each sample by its SCLC-score and created a ‘signature score’ that discriminates between the three subtypes with high predictive performance. The POU2F3 signature in particular separates plasma samples of this uncommon subtype from the others even in cases with very low SCLC-score (FIG. 5C-5D, 10B-10C). We therefore defined a decision tree multi-step classifier to classify plasma samples to the three subtypes above where samples are initially evaluated against the POU2F3 signature and subsequently against the other two signatures (FIG. 5E). On our training data this tree correctly classifies 47 out of 53 samples, including 25 samples with low SCLC-score that were not used in defining the genomic signatures.

These findings suggest that cfChIP-seq can fill an unmet need of molecularly classifying SCLC into transcriptomic subsets of potential therapeutic relevance in a limited-invasive manner directly from plasma.

TABLE 1
ACSL1 informative genomic loci. Chromosomal locations are given with respect to
human genome build hg19. FDR-corrected q-values are provided for significance.

| | | | | Gene (if within a gene body | |
|---|---|---|---|---|---|
| # | Chromosome | start | end | or gene promoter) | q.value |
| 1 | chr19 | 13734801 | 13735000 | CACNA1A | 0.0000055 |
| 2 | chr12 | 103355001 | 103356200 | . | 0.0000177 |
| 3 | chr18 | 56815201 | 56816200 | NA | 0.0000177 |
| 4 | chr10 | 2813601 | 2818600 | NA | 0.0000237 |
| 5 | chr21 | 47167601 | 47168200 | PCBP3 | 0.0000889 |
| 6 | chr18 | 42477001 | 42477600 | . | 0.0000906 |
| 7 | chr7 | 155198201 | 155203200 | NA | 0.0000992 |
| 8 | chr14 | 36981201 | 36983200 | SFTA3 | 0.0000992 |
| 9 | chr18 | 40856001 | 40857800 | SYT4 | 0.0000992 |
| 10 | chr14 | 36978001 | 36980000 | SFTA3 | 0.0001187 |
| 11 | chr14 | 36985601 | 36986400 | BX161496 | 0.0002712 |
| 12 | chr9 | 116059601 | 116061000 | NA | 0.0002935 |
| 13 | chr12 | 53685801 | 53687200 | NA | 0.0002935 |
| 14 | chr18 | 76574201 | 76575200 | . | 0.0002935 |
| 15 | chr1 | 240286801 | 240287800 | . | 0.0003870 |
| 16 | chr3 | 178277001 | 178278800 | KCNMB2 | 0.0004098 |
| 17 | chr14 | 36983201 | 36985200 | SFTA3 | 0.0004993 |
| 18 | chr2 | 166095801 | 166097800 | SCN2A | 0.0004994 |
| 19 | chr14 | 36980001 | 36981200 | SFTA3 | 0.0006706 |
| 20 | chr13 | 108516001 | 108516800 | FAM155A | 0.0008458 |
| 21 | chr18 | 31323201 | 31324400 | . | 0.0011586 |
| 22 | chr18 | 70359601 | 70360200 | . | 0.0015124 |
| 23 | chr18 | 56813401 | 56815200 | NA | 0.0015989 |
| 24 | chr8 | 2150601 | 2151600 | AX747124 | 0.0017683 |
| 25 | chr2 | 166055201 | 166057000 | . | 0.0018535 |
| 26 | chr4 | 8011601 | 8012400 | ABLIM2 | 0.0018535 |
| 27 | chr1 | 213819801 | 213824800 | NA | 0.0021273 |
| 28 | chr18 | 31322201 | 31323200 | NA | 0.0021273 |
| 29 | chr2 | 166057001 | 166057800 | NA | 0.0022090 |
| 30 | chr1 | 241371601 | 241372600 | RGS7 | 0.0029670 |
| 31 | chr14 | 36964801 | 36969800 | NA | 0.0057565 |
| 32 | chr8 | 40754601 | 40755600 | ZMAT4 | 0.0077657 |
| 33 | chr22 | 49279201 | 49284200 | NA | 0.0077657 |
| 34 | chr16 | 73033801 | 73034800 | . | 0.0100156 |
| 35 | chr9 | 117191001 | 117192800 | NA | 0.0199150 |
| 36 | chr9 | 96455001 | 96460000 | NA | 0.0222658 |
| 37 | chr3 | 170184801 | 170187200 | BC039437 | 0.0329016 |
| 38 | chr18 | 31317801 | 31322200 | NA | 0.0334124 |
| 39 | chr12 | 114289001 | 114294400 | NA | 0.0644497 |
| 40 | chr4 | 113970801 | 113971400 | ANK2 | 0.0696419 |
| 41 | chr1 | 51439601 | 51440000 | . | 0.1721682 |
| 42 | chr10 | 16715001 | 16720000 | NA | 0.1721682 |
| 43 | chr6 | 169845801 | 169846400 | . | 0.1949554 |
| 44 | chr7 | 55506401 | 55507600 | . | 0.2637918 |
| 45 | chr14 | 37133001 | 37133200 | PAX9 | 0.6433609 |

TABLE 2
NEUROD1 informative genomic loci. Chromosomal locations are given with respect to
human genome build hg19. FDR-corrected q-values are provided for significance.

| | | | | Gene (if within a gene body | |
|---|---|---|---|---|---|
| # | Chromosome | start | end | or gene promoter) | q.value |
| 1 | chr7 | 4204401 | 4209400 | NA | 2.97E−09 |
| 2 | chr7 | 129417401 | 129417800 | NA | 0.00648778 |
| 3 | chr17 | 47547601 | 47548000 | . | 0.01343497 |
| 4 | chr2 | 23699601 | 23702600 | NA | 0.03931537 |
| 5 | chr14 | 66543601 | 66548600 | NA | 0.03931537 |
| 6 | chr15 | 79574601 | 79575000 | ANKRD34C | 0.03931537 |
| 7 | chr20 | 21378401 | 21380200 | NA | 0.03931537 |
| 8 | chr19 | 41039001 | 41044000 | NA | 0.0440929 |
| 9 | chr20 | 20232001 | 20233000 | NA | 0.06703899 |
| 10 | chr16 | 2511601 | 2513000 | C16orf59 | 0.06768626 |
| 11 | chr9 | 86426601 | 86430000 | NA | 0.15737288 |
| 12 | chr16 | 89238601 | 89241200 | CDH15 | 0.15737288 |
| 13 | chr19 | 42495401 | 42497000 | ATP1A3 | 0.16061865 |
| 14 | chr11 | 45529401 | 45533000 | NA | 0.20238844 |
| 15 | chr13 | 100505401 | 100506400 | . | 0.27623803 |
| 16 | chr22 | 30108401 | 30114000 | NA | 0.35978335 |
| 17 | chr2 | 121845001 | 121848000 | AC067960.1 | 0.55763485 |
| 18 | chr13 | 100504401 | 100505400 | NA | 0.63988008 |
| 19 | chr20 | 588601 | 589800 | TCF15 | 0.63988008 |
| 20 | chr19 | 19372801 | 19373000 | HAPLN4 | 0.70043245 |
| 21 | chr1 | 155356001 | 155361000 | NA | 0.8112726 |
| 22 | chr2 | 154183801 | 154186400 | NA | 0.8112726 |
| 23 | chr2 | 182539401 | 182542400 | NEUROD1 | 0.8112726 |
| 24 | chr5 | 67113401 | 67114000 | RP11-83M16.6 | 0.8112726 |
| 25 | chr6 | 41343401 | 41344400 | NA | 0.8112726 |
| 26 | chr11 | 64409601 | 64409800 | NRXN2 | 0.8112726 |
| 27 | chr14 | 103216801 | 103220200 | NA | 0.8112726 |
| 28 | chr17 | 43043801 | 43044400 | C1QL1 | 0.8112726 |
| 29 | chr7 | 27237601 | 27238200 | HOXA13 | 0.91115827 |
| 30 | chr7 | 156809801 | 156810800 | NA | 0.91115827 |
| 31 | chr11 | 31828201 | 31829800 | PAX6 | 0.91115827 |
| 32 | chr14 | 23361001 | 23365800 | NA | 0.91115827 |
| 33 | chr19 | 34769201 | 34774200 | NA | 0.91115827 |
| 34 | chr1 | 244360401 | 244365400 | NA | 0.92427242 |
| 35 | chr8 | 137012001 | 137017000 | NA | 0.92427242 |
| 36 | chr8 | 140254601 | 140259600 | NA | 0.92427242 |
| 37 | chr11 | 74726201 | 74730800 | NA | 0.92427242 |
| 38 | chr14 | 74725401 | 74725600 | . | 0.92427242 |
| 39 | chr1 | 181287801 | 181288200 | . | 0.99271104 |

TABLE 3
POU2F3 informative genomic loci. Chromosomal locations
are given with respect to human genome build hg19.
FDR-corrected q-values are provided for significance.

| | | | | Gene (if within a gene body | |
|---|---|---|---|---|---|
| # | Chromosome | start | end | or gene promoter) | q.value |
| 1 | chr21 | 38074001 | 38076000 | SIM2 | 6.15E−19 |
| 2 | chr6 | 33084601 | 33085200 | . | 2.57E−18 |
| 3 | chr11 | 67441001 | 67442200 | ALDH3B2 | 2.57E−18 |
| 4 | chr9 | 140960601 | 140965600 | NA | 3.17E−17 |
| 5 | chr1 | 245507401 | 245509200 | . | 1.81E−15 |
| 6 | chr10 | 86522001 | 86523000 | . | 7.74E−15 |
| 7 | chr7 | 36446401 | 36448600 | ANLN | 5.53E−14 |
| 8 | chr17 | 36810001 | 36811600 | . | 4.38E−13 |
| 9 | chr10 | 37232201 | 37232600 | . | 1.34E−10 |
| 10 | chr21 | 15538001 | 15543000 | NA | 1.76E−10 |
| 11 | chr4 | 154217001 | 154217800 | . | 7.27E−10 |
| 12 | chr18 | 66457601 | 66458800 | . | 0.0000078 |
| 13 | chr12 | 8286001 | 8288400 | . | 0.0006095 |
| 14 | chr16 | 2974601 | 2975800 | . | 0.0013805 |
| 15 | chr4 | 174529601 | 174530200 | . | 0.0027208 |
| 16 | chr17 | 44087201 | 44088400 | . | 0.002993 |
| 17 | chr8 | 128426201 | 128429200 | POU5F1B | 0.0051249 |
| 18 | chr18 | 74061601 | 74062600 | NA | 0.0082606 |
| 19 | chr8 | 48091001 | 48092000 | . | 0.010202 |
| 20 | chr4 | 71925001 | 71926200 | . | 0.0104593 |
| 21 | chr7 | 114670601 | 114671200 | . | 0.0108433 |
| 22 | chr12 | 16535801 | 16536200 | MGST1 | 0.0114698 |
| 23 | chr18 | 39211401 | 39211800 | KC6 | 0.0177769 |
| 24 | chr4 | 184667601 | 184667800 | . | 0.0206614 |
| 25 | chr1 | 2315601 | 2317600 | MORN1 | 0.0228051 |
| 26 | chr4 | 156128801 | 156129600 | NPY2R | 0.0267661 |
| 27 | chr5 | 38445401 | 38446800 | EGFLAM | 0.0274198 |
| 28 | chr12 | 54387201 | 54388000 | MIR196A2 | 0.0398986 |
| 29 | chr9 | 140918801 | 140919800 | CACNA1B | 0.04465 |
| 30 | chr10 | 133776401 | 133777800 | NA | 0.0465039 |
| 31 | chr12 | 54381001 | 54382800 | HOXC10 | 0.0541979 |
| 32 | chr3 | 170601801 | 170602800 | . | 0.0551732 |
| 33 | chr22 | 22901201 | 22901800 | PRAME; LOC648691 | 0.0551732 |
| 34 | chr1 | 178915001 | 178915400 | . | 0.0709365 |
| 35 | chr22 | 35939401 | 35939800 | RASD2 | 0.0709365 |
| 36 | chr11 | 6164801 | 6165400 | . | 0.0948594 |
| 37 | chr6 | 1595001 | 1596200 | NA | 0.0986849 |
| 38 | chr2 | 6911801 | 6913000 | LINC00487 | 0.1076044 |
| 39 | chr10 | 729201 | 730400 | . | 0.1076044 |
| 40 | chr20 | 61467801 | 61468200 | COL9A3 | 0.1093574 |
| 41 | chr4 | 155666201 | 155666800 | LRAT | 0.1171573 |
| 42 | chr8 | 48676601 | 48677800 | . | 0.1235607 |
| 43 | chr8 | 103549601 | 103550800 | . | 0.1235607 |
| 44 | chr10 | 45693001 | 45694000 | NA | 0.1235607 |
| 45 | chr10 | 65880001 | 65880600 | . | 0.1235607 |
| 46 | chr10 | 126295001 | 126296800 | LHPP | 0.1235607 |
| 47 | chr4 | 65138001 | 65138600 | . | 0.1446326 |
| 48 | chr18 | 40866001 | 40866400 | . | 0.1453835 |
| 49 | chr6 | 1594001 | 1595000 | . | 0.1712276 |
| 50 | chr17 | 25335801 | 25336800 | NA | 0.1875345 |
| 51 | chr18 | 1521801 | 1522200 | . | 0.1912004 |
| 52 | chr4 | 176592601 | 176593200 | . | 0.2010458 |
| 53 | chr10 | 97174201 | 97176000 | SORBS1 | 0.2010458 |
| 54 | chr12 | 38549201 | 38553000 | NA | 0.2010458 |
| 55 | chr17 | 60764601 | 60767600 | MRC2 | 0.2019779 |
| 56 | chr18 | 60803801 | 60805000 | . | 0.2023649 |
| 57 | chr5 | 28286801 | 28287600 | CTD-2061E9.1 | 0.2065424 |
| 58 | chr5 | 32500401 | 32501800 | . | 0.2240014 |
| 59 | chr17 | 10705801 | 10708000 | LINC00675 | 0.2320636 |
| 60 | chr17 | 38166601 | 38168800 | NA | 0.2320636 |
| 61 | chr18 | 48663201 | 48664400 | . | 0.2327199 |
| 62 | chr2 | 171838801 | 171840600 | NA | 0.2369754 |
| 63 | chr2 | 242127601 | 242128600 | ANO7 | 0.2369754 |
| 64 | chr3 | 122630601 | 122631600 | NA | 0.2394191 |
| 65 | chr17 | 70119201 | 70122200 | AL833139; SOX9 | 0.2470781 |
| 66 | chr11 | 120112801 | 120118000 | NA | 0.2478654 |
| 67 | chr1 | 14927401 | 14928200 | KAZN | 0.2658589 |
| 68 | chr18 | 34897401 | 34902400 | NA | 0.2658589 |
| 69 | chr11 | 56947801 | 56948400 | LRRC55 | 0.2668003 |
| 70 | chr11 | 120006401 | 120008800 | TRIM29 | 0.2668003 |
| 71 | chr8 | 53475401 | 53476800 | FAM150A | 0.301268 |
| 72 | chr12 | 38544201 | 38549200 | NA | 0.3331603 |
| 73 | chr22 | 38379201 | 38380200 | SOX10 | 0.3374236 |
| 74 | chr12 | 54349201 | 54350200 | NA | 0.3454906 |
| 75 | chr18 | 14747801 | 14748600 | ANKRD30B | 0.3454906 |

### Example 6: Elevated Liver-Derived cfDNA in AIH Plasma Samples

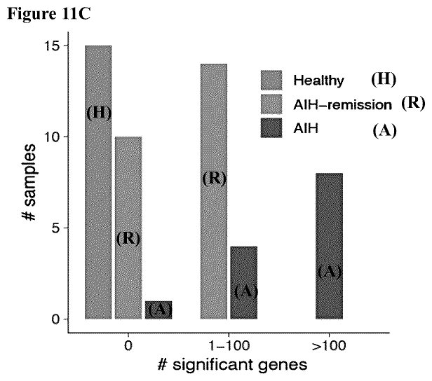

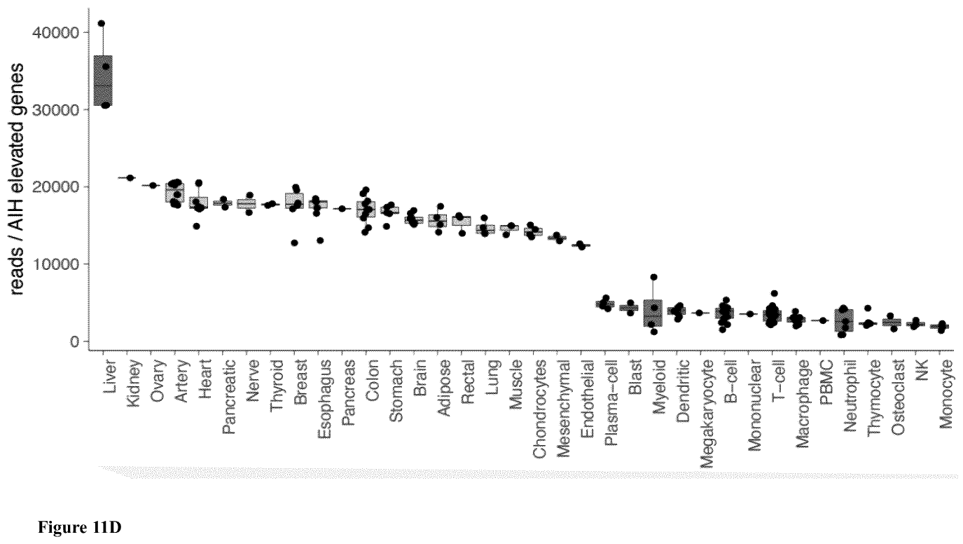

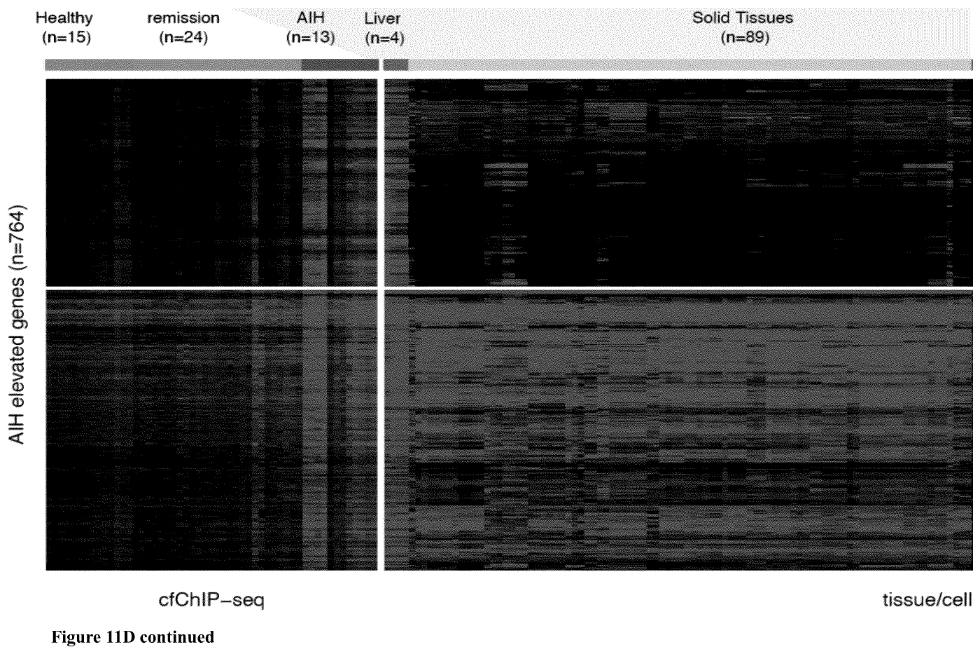

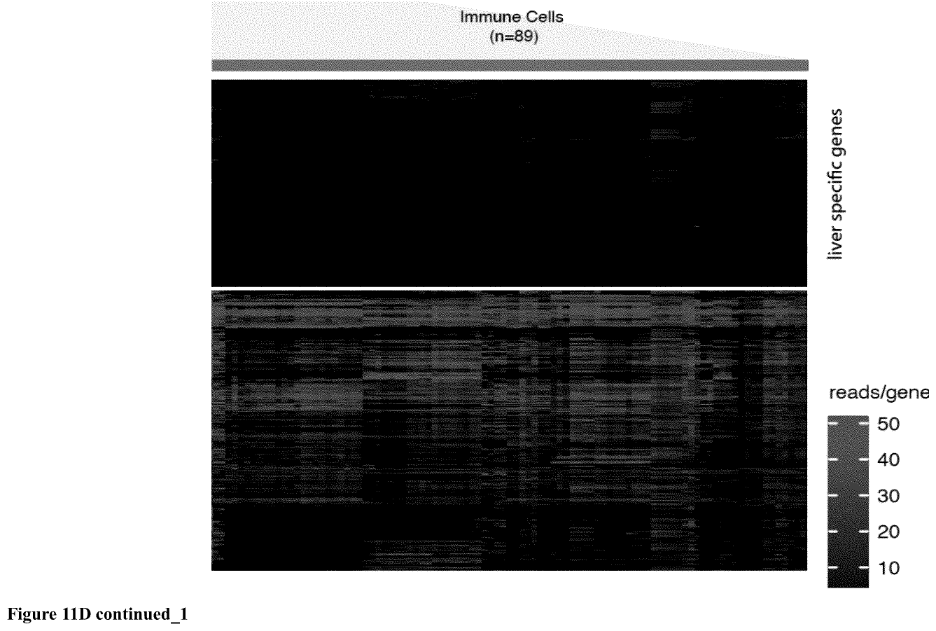

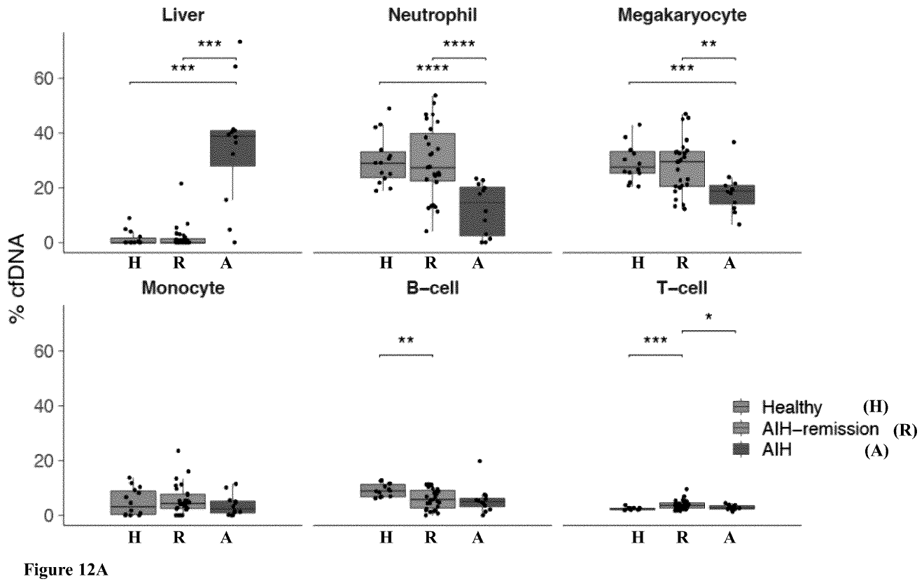

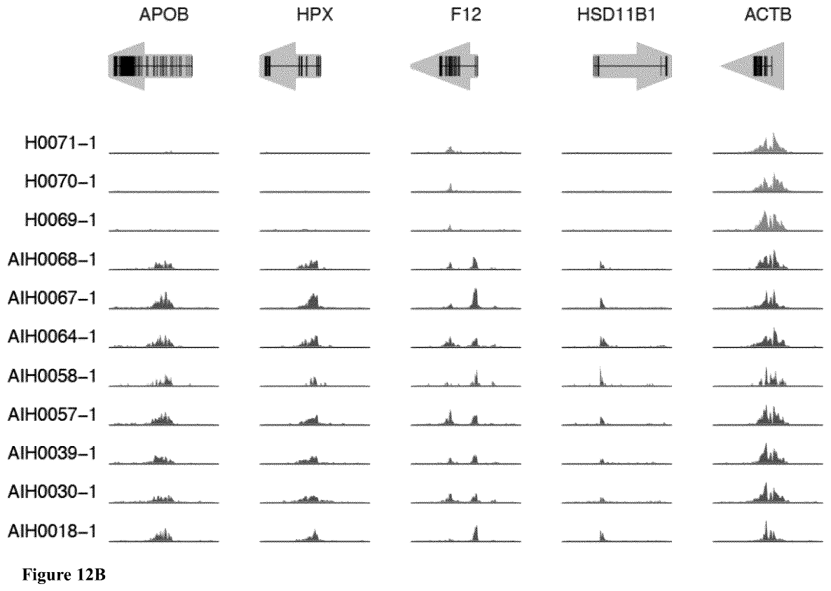

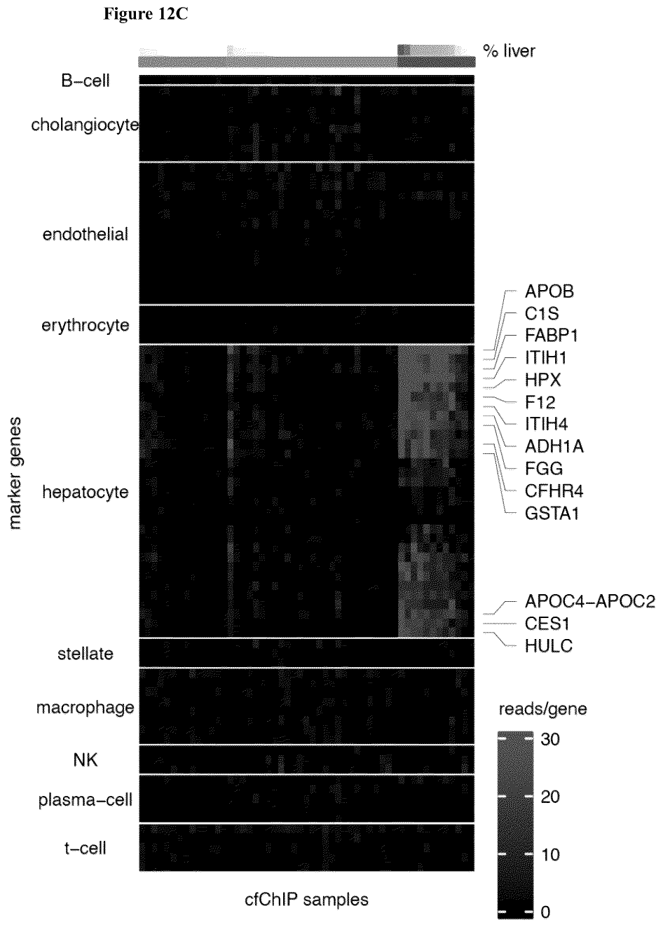

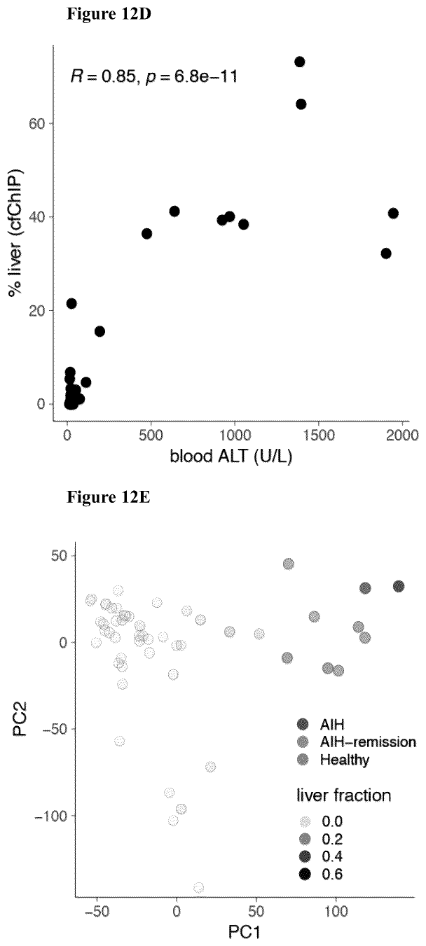

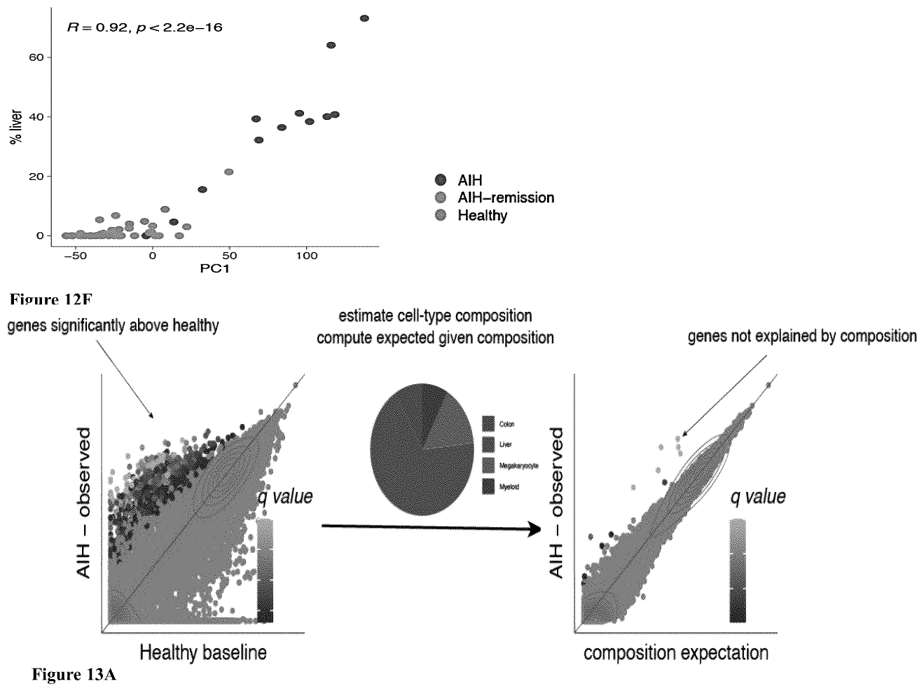

The method of identifying disease and disease subtype markers for cfChIP-seq was extended to a second disease-autoimmune hepatitis (AIH). In this disease, the objective is to assist diagnosis and monitoring response to treatment. To this end we used cfChIP-seq with H3K4me3-specific antibody on 37 plasma samples from pediatric patients with autoimmune hepatitis (n=27 patients)—either at diagnosis, with elevated liver transaminases or in biochemical remission (ALT and AST liver enzymes within the normal range) under immunosuppressive therapy. As control, we also included an additional cohort of 14 self-reported healthy donors (six children and eight adults) and a cohort of 58 samples from 56 patients with other liver diseases (FIG. 11A).

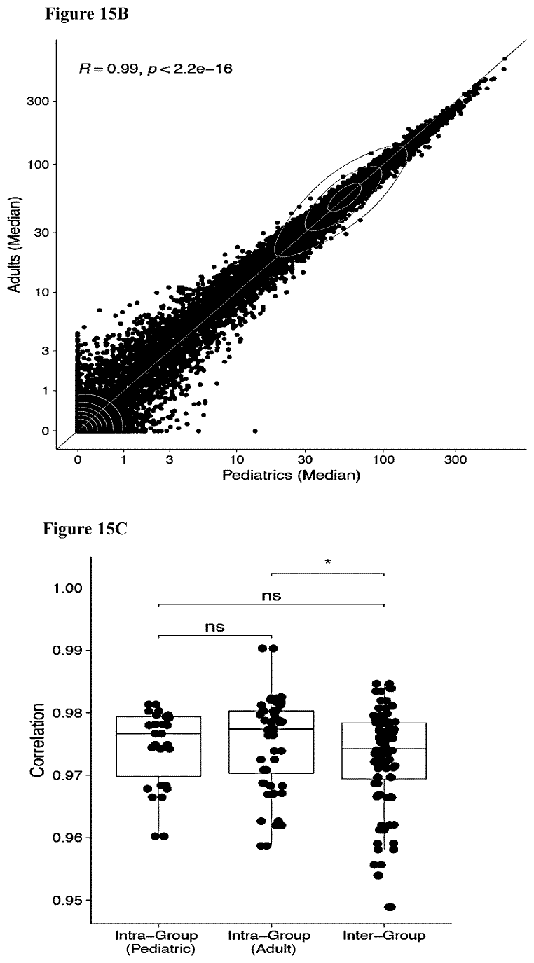

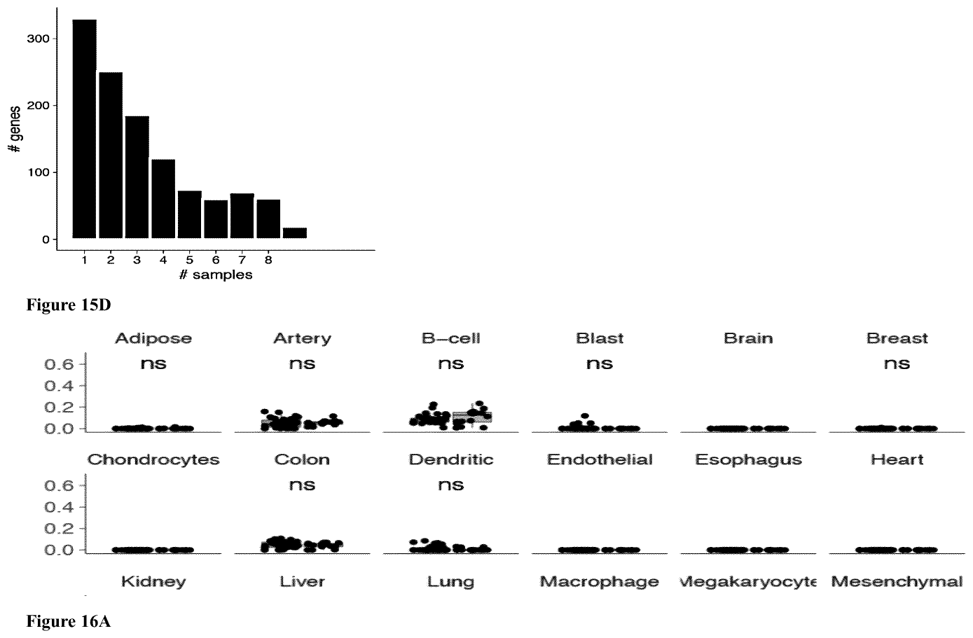

For quality control we examine the yield of the assay and its specificity. The average yield of the cfChIP-seq samples was 2.8 and 1.4 million unique reads for the AIH and healthy samples respectively (FIG. 15A; presumably reflecting elevated cfCDNA levels in the AIH samples. The specificity of cfChIP-seq is defined as the proportion of reads that map to gene promoters vs. reads that are non-specific background. The average specificity of the samples is 70% (Methods).

The self reported healthy control cohort consists of samples from children and adults. The means of the two groups were highly correlated (R=0.99), and individual samples were also highly correlated (R>0.95, median R=0.975;) and were therefore treated as one unified control group for downstream analysis.

The results of cfChIP-seq are analyzed at the level of genes. Briefly, reads are mapped to the genome and the number of normalized reads mapping to every gene's TSS regions was computed, resulting in gene counts resembling RNA-seq transcription counts (Methods). We then compared the gene counts of plasma samples from AIH patients to a healthy baseline reference (Methods) and found hundreds of genes that were significantly increased in AIH patients with active disease, many of which were shared among several samples. In plasma samples from patients in remission, we observed a smaller group of genes elevated compared to healthy, and some samples seemed identical to healthy plasma with no genes significantly elevated (FIG. 11B-11C, 15B).

To identify the tissues and cell types that contribute to the elevated gene signal in AIH patients, we compared the profile of these genes to a comprehensive reference atlas of 182 H3K4me3 ChIP-seq samples from 36 tissues and cell-types, including solid tissues and immune cells. Examining the set of genes which were significantly elevated in at least 3 AIH samples in the reference data, exhibits a low coverage in the immune cells which are the main source of cfDNA in healthy individuals. A subset of the genes had some coverage in all solid tissues, and the majority of genes are marked by H3K4me3 only in the liver samples (FIG. 1D). Enrichment tests of this gene set found a strong enrichment for the liver (EnrichR human gene atlas q<10-80; gene overlap 140/618), reestablishing the identification of the liver as a major source of cfDNA in AIH samples.

### Example 7: CfChIP-Seq Recovers AIH Cell-Free DNA Cell-of-Origin

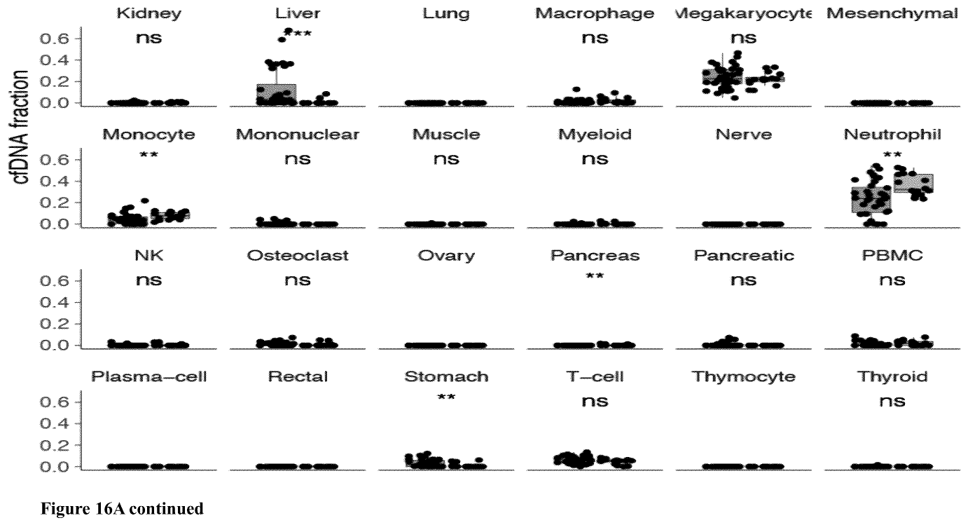

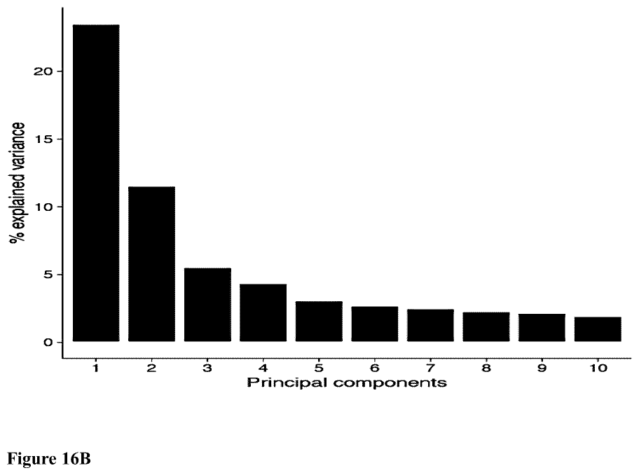

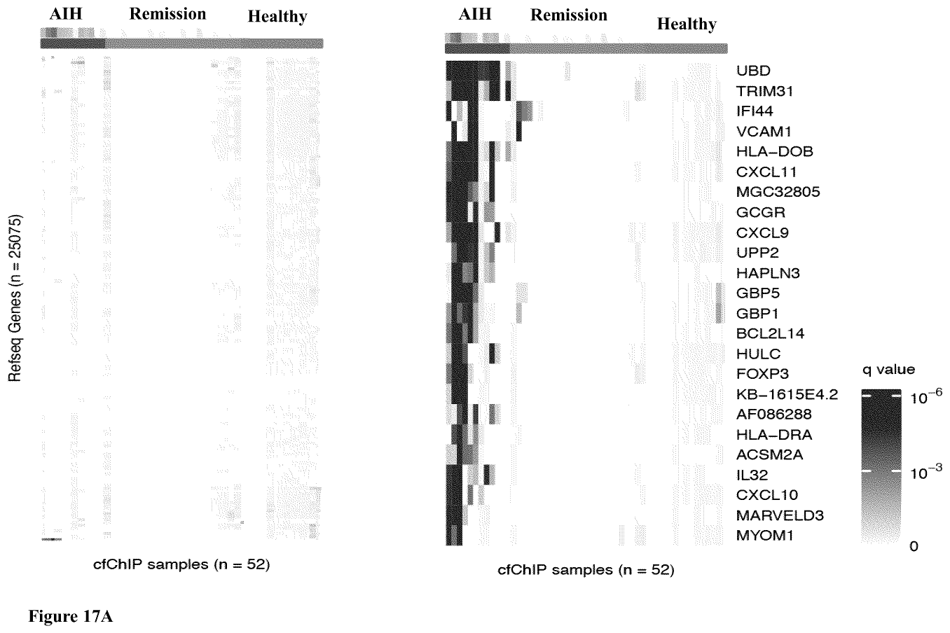

To quantify the relative contribution of liver-derived cfDNA in the circulation and achieve a systematic view of other tissues contributing to the circulation, we used a linear regression deconvolution of samples to their composing cell-types (Methods). Examining the results, we find that in healthy donors, the major components of the cfDNA are the peripheral blood mononuclear cell (neutrophils, megakaryocytes, B cells and monocytes) which is in agreement with previous studies, while the liver constitutes less than 1% of the cfDNA an average. In contrast, in samples of AIH patients with active disease, the liver accounts for 15%-65% of the cfDNA, and increased levels are observed also in some of the patients in remission (t-test P=0.0002 and 0.01 in the active and remission AIH samples respectively). An additional, more subtle elevation is observed also in the T cell fraction of some AIH samples (FIG. 2A, 16A). Note that the estimated fractions represent the relative contribution of the tissues to the circulation, and not the absolute cell death of these tissues. Thus, an increase in liver fraction must be compensated by reduction of fractions of other tissues even if their absolute levels remain the same (FIG. 16B).

To further test whether cfChIP-seq can provide clues as to the AIH specific cell-type of origin within the liver, we used gene signatures derived from a single cell RNA-seq liver atlas, since no such ChIP-seq data exist. Across the ˜10 liver cell-types examined, including hepatocyte, cholangiocyte, endothelial and immune cells, the AIH samples are enriched specifically for hepatocyte marker genes such as HPX (hemopexin), F12 (coagulation factor XII) and GPD1 (Glycerol-3-Phosphate Dehydrogenase 1) (FIG. 2B-2C). The remarkably positive correlation of the hepatocyte marker genes and the liver fraction (R=0.98; FIG. 16C) further corroborates the finding that the hepatocytes are indeed the major source of liver cfDNA in AIH plasma samples.

Comparison of the estimated liver fraction to the liver alanine transferase (ALT) levels measured in time-matched blood samples, displays a good agreement between the two modalities despite the differences in half-life of these analytes 15,16 (R=0.87; p=7.3-12; FIG. 2D). When performing principal component analysis (PCA) of the samples (Methods) the first principal component (which accounts for 25% of variability) is highly correlated with estimated liver fraction (R=0.93, p<1×1015. FIG. 2E-2F, 16D).

Overall, these findings show that the predominant abnormality in AIH circulating DNA is an increase in hepatocyte contribution. The auto-immune nature of the disease suggests that this increase is due to immune attack on hepatocytes.

### Example 8: CfChIP-Seq Identifies Hepatocyte Immune Response

Histone modifications are intimately related to the activity of RNA polymerase. H3K4me3 in particular, is a histone modification associated with transcription initiation and transcriptional pause-release. Thus levels of H3K4me3 are representative of the amount of such events in the cells that contribute to the circulating cfDNA pool. AIH is characterized by a complex process that involves activation of CD4+ effector and regulatory T-cells, cytokine and chemokine production and more. We thus seek to explore whether cfChIP-seq can detect such processes in AIH plasma samples.

To distinguish changes within specific cell-types on the background of changes in cell-type composition, we used the following strategy: First, we used deconvolution to estimate cell-type composition of a sample. We then construct composition-informed reference for the specific sample taking into account the relative composition and the estimate of mean and variance gene levels in each cell type. Comparing this reference to the observed values we can identify genes that are significantly above or below the revised reference (FIG. 3A; Methods).

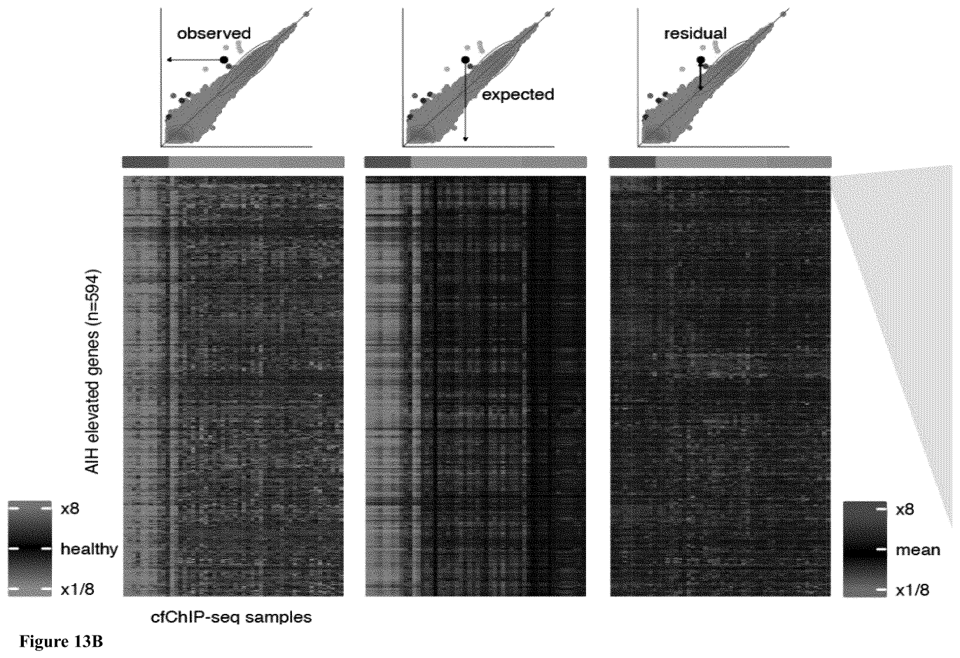

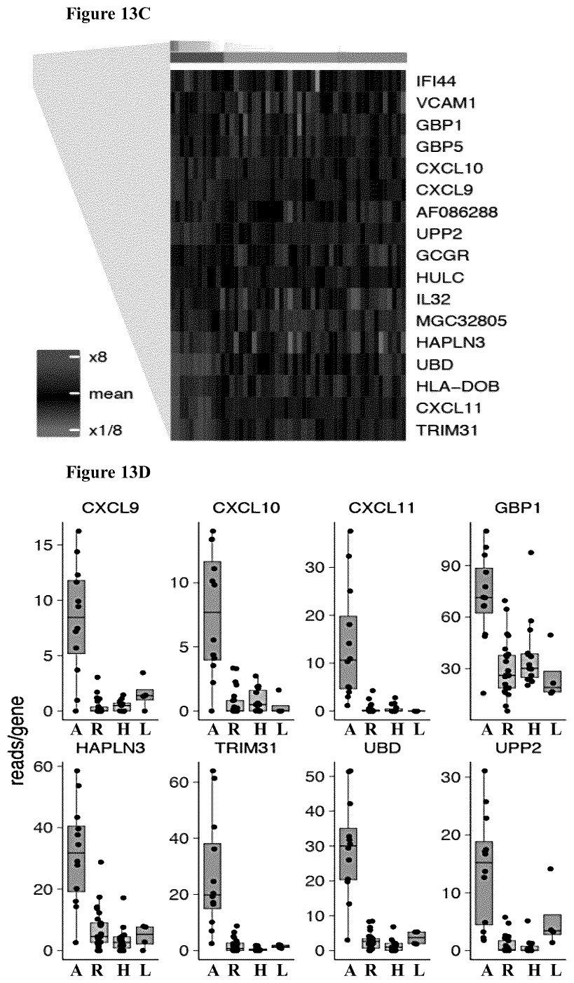

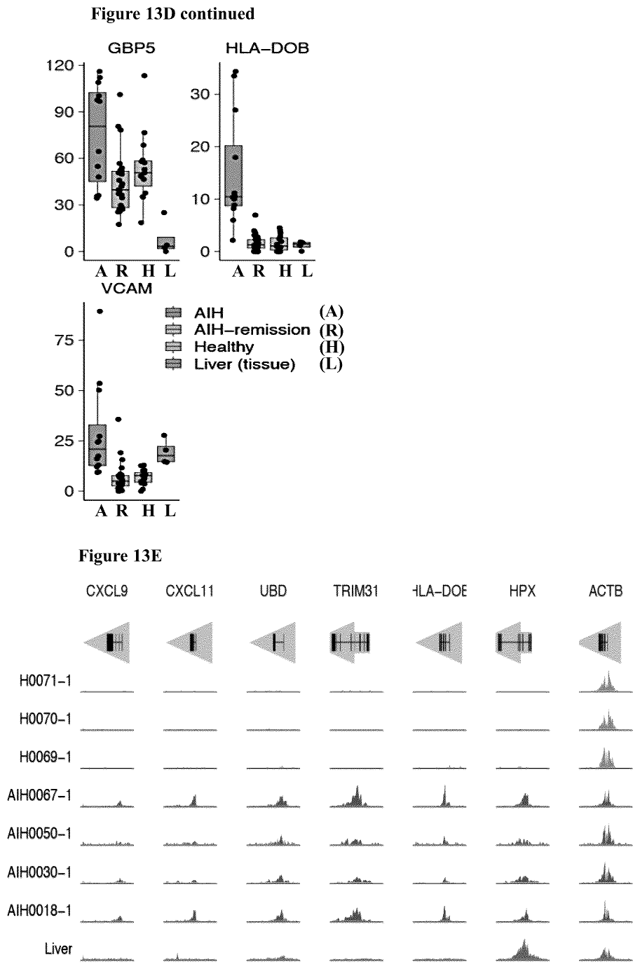

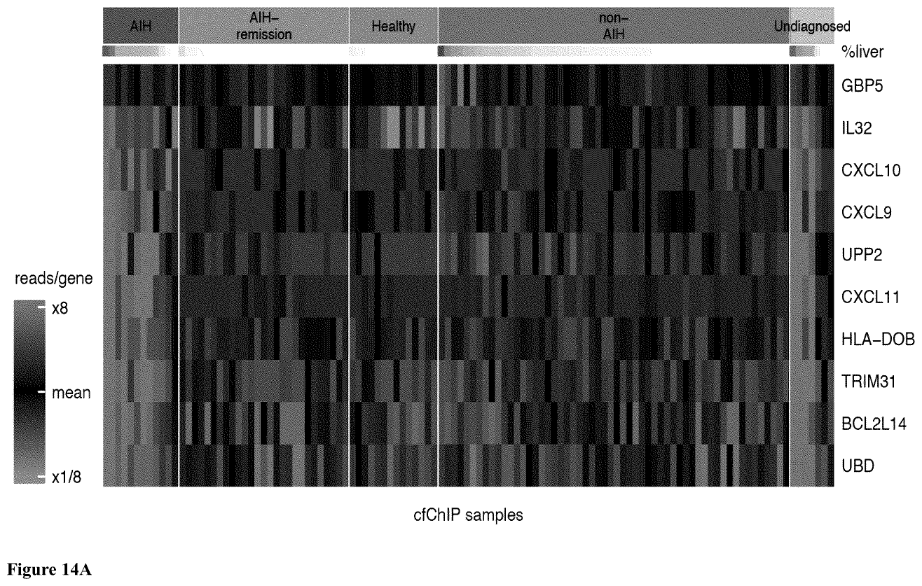

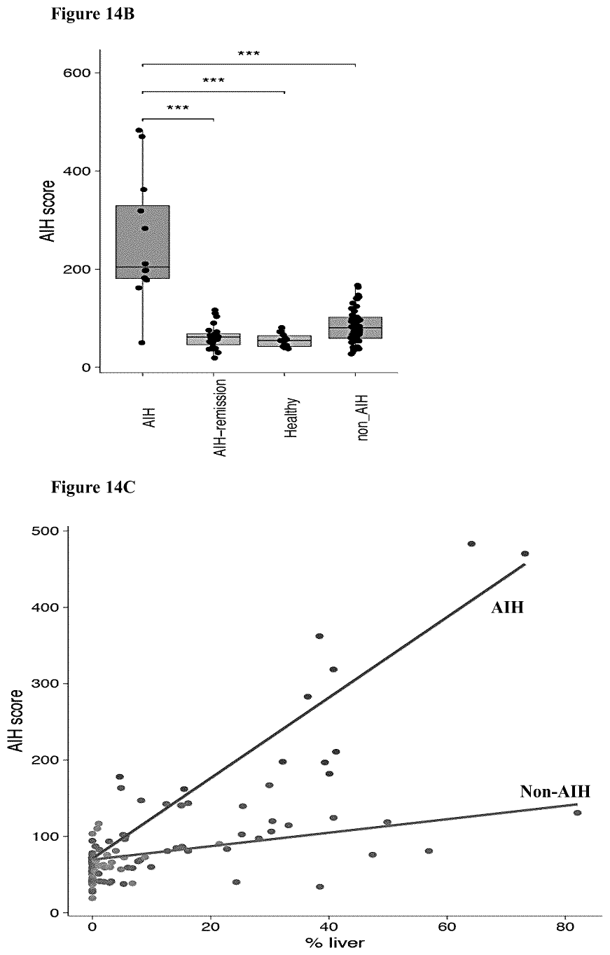

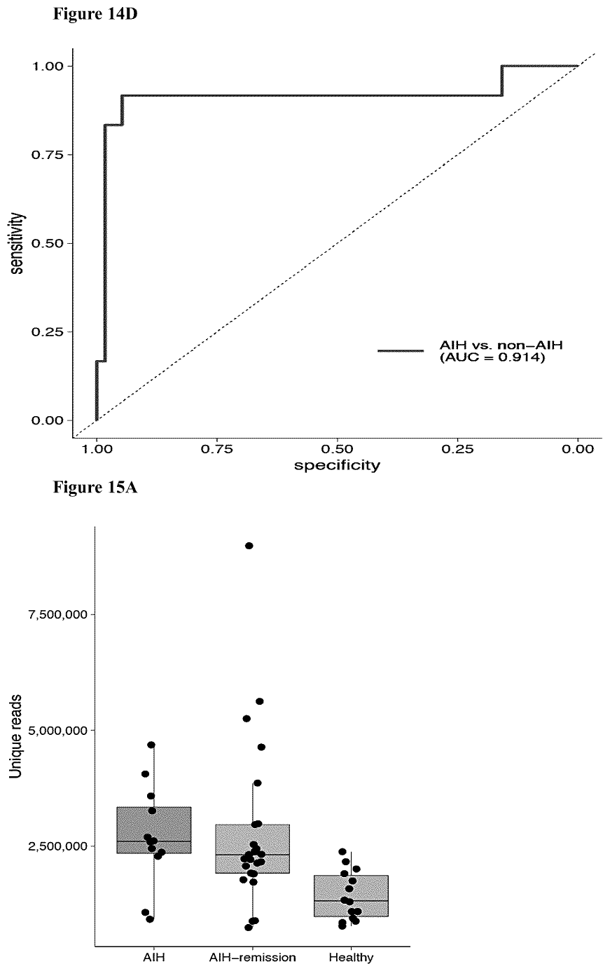

Applying this model to the AIH samples we find that the vast majority of observed gene counts (99.98%) do not significantly deviate from the composition-informed reference (FIG. 16A). Focusing on the 774 genes with significantly elevated signal in the AIH samples compared to healthy, we find that here too the majority of genes (97%) do not significantly deviate from composition-informed reference suggesting that they reflect the normal transcription patterns of the liver (FIG. 13B). However, a close inspection of this group reveals a set of genes with coverage significantly above expected in several samples (FIG. 3C). This group includes the CXCL9-11 (C-X-C motif chemokine ligand) genes, which are expressed in inflamed hepatocytes and play a role in the AIH immune response and liver fibrosis. Other genes that are significantly above expected are UBD (Ubiquitin D) and TRIM31 (Tripartite Motif Containing 31) which are induced by pro inflammatory cytokines and HLA-DOB (Major histocompatibility complex, class II, Do Beta) which was identified as affecting the occurrence and development of hepatitis B (HBV) but not reported in the context of AIH. The interferon induced genes GBP1 and GBP5 that were reported to induce liver injury and inflammation were also significantly above expected. Another gene identified is HULC (highly upregulated liver cancer) which is expressed in normal hepatocytes but strongly induced in hepatocellular carcinoma and HBV infection and involved in inflammatory injury in rats with cirrhosis. Importantly, many of these genes, which have high coverage in AIH samples, carry no H3K4me3 signal in normal liver nor in any of the other tissues represented in the reference atlas, indicating that they reflect activation of an abnormal transcription program occurring in the patients with AIH (FIG. 3D-E; 17B).

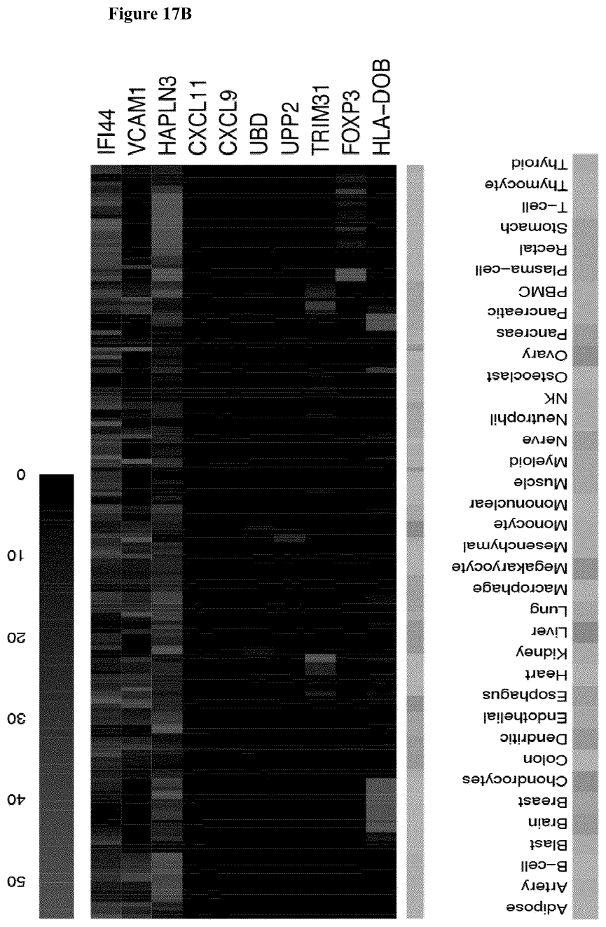

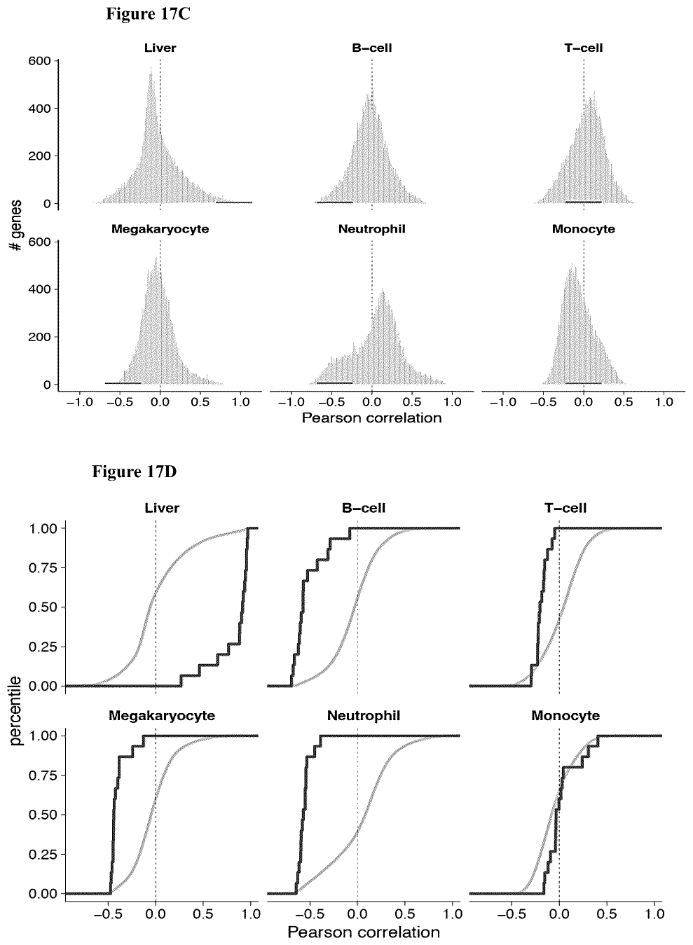

To rule out the possibility that these genes reflect a transcriptional program in other cell types other than the liver, we tested the correlation between the gene levels and the estimated fraction across all tissues composing the samples. This analysis revealed that these genes are positively correlated to the estimated liver fraction in the AIH samples and negatively correlated to all other tissues (FIG. 17B-17C). We conclude that the activity of these genes is strictly coupled to hepatocyte death, most likely reflecting transcription in the hepatocyte cells of patients with AIH.

Taken together, these results demonstrate that plasma H3K4me3 cfChIP-seq reliably identifies a hepatocyte immune process taking place in the AIH patients.

### Example 9: Plasma Based Classifier for AIH Diagnosis and Monitoring

We next sought to test whether this realization can be utilized in the clinical setting in assisting the diagnosis and treatment management of patients with AIH.

One of the most prominent findings described so far indicates an elevation of liver derived cfDNA in patients with AIH compared to healthy control. Indeed, the single attribute of liver fraction suffices for discriminating between these two groups. In the clinical setting, however, the challenge is often differentiating AIH from other liver diseases and conditions that involve liver damage, such as drug induced liver injury or infections. To identify signals specific to AIH and to design a classifier aimed at distinguishing AIH from other diseases, we made use of previously published cfChIP-seq samples 6 (n=18) with elevated liver derived cfDNA from patients with various diseases. We performed cfChIP-seq on two additional cohorts of adult (n=30) and pediatric (n=10) patients with various liver-related diseases. The adult cohort includes patients with nonalcoholic steatohepatitis (NASH), fatty liver, hepatitis B and C, drug induced liver injury, Cholestatic liver disease, primary biliary cholangitis and patients that underwent liver transplant. The pediatric cohort included patients that underwent liver biopsy due to elevated liver enzymes and were diagnosed with metabolic diseases, fatty liver, hypobetalipoproteinemia and with non-specific finding in the liver biopsies that were not compatible with AIH or any other disease.

As above, we neutralize the variable relative contribution of different tissues by computing residual signals—the differences between observed signal and the expected signal given the specific cell-type composition of the sample. Comparing the residual signal of the non-AIH and AIH samples over the group of genes that significantly deviate from the composition-informed reference described above, we find that 15 of the 29 genes are significantly elevated in the AIH group (t-test, q<0.1 after false discovery rate (FDR) correction). Many of these genes lack signal completely in almost all non-AIH samples, supporting the role of these genes in an AIH unique immune response, as described hereinabove. Based on the differential genes, we define an ‘AIH score’ as the cumulative signal of the genes elevated in the AIH groups (FIG. 4A). After computing this score of all samples, a clear distinction is apparent between the AIH and non-AIH samples (FIG. 4B). Testing the effect of the liver derived cfDNA fraction on the ‘AIH score’ exhibits a linear relationship between the two in the AIH group. In the non-AIH group in contrast, this phenomenon is much less pronounced, reflecting the fact that this signature captures a transcription program typically inactive in the liver (FIG. 4C). Finally, using a classifier based on the AIH-score demonstrates the capability to accurately discriminate between the AIH and non-AIH plasma samples (AUC=0.914; FIG. 4D).

These results suggest that cfChIP-seq can fill an unmet need in assisting AIH diagnosis in a non-invasive manner directly from plasma.

### Example 10

The methods of the invention are carried out on other types of cancer and markers for different subtypes of various cancers are produced. Healthy subjects and subjects that suffer from breast cancer provide ChIP-Seq reads from blood samples. Tumor load is calculated and markers from various subtypes, such as Ductal carcinoma, Lobular carcinoma, Inflammatory breast cancer, and triple negative breast cancer are generated. Multigene signatures are generated that allow for breast cancer subtyping.

Healthy subjects and subjects that suffer from non-small cell lung cancer provide ChIP-Seq reads from blood samples. Tumor load is calculated and markers from various subtypes, such as Squamous cell carcinoma, Adenocarcinoma, and Large cell carcinoma are generated. Multigene signatures are generated that allow for non-small cell lung cancer subtyping.

Healthy subjects and subjects that suffer from prostate cancer provide ChIP-Seq reads from blood samples. Tumor load is calculated and markers from various subtypes, such as Adenocarcinoma, Small cell carcinoma, Ductal adenocarcinoma, and Prostatic intraepithelial neoplasia are generated. Multigene signatures are generated that allow for prostate cancer subtyping.

Healthy subjects and subjects that suffer from leukemia provide ChIP-Seq reads from blood samples. Tumor load is calculated and markers from various subtypes, such as Acute lymphoblastic leukemia (ALL), Chronic lymphocytic leukemia (CLL), Acute myeloid leukemia (AML) and Chronic myeloid leukemia (CML) are generated. Multigene signatures are generated that allow for leukemia subtyping.

Healthy subjects and subjects that suffer from lymphoma provide ChIP-Seq reads from blood samples. Tumor load is calculated and markers from various subtypes, such as Hodgkin lymphoma, Non-Hodgkin lymphoma (NHL), Diffuse Large B-cell Lymphoma, Follicular Lymphoma, Mantle Cell Lymphoma, Burkitt Lymphoma, and Marginal Zone Lymphoma are generated. Multigene signatures are generated that allow for lymphoma subtyping.

Healthy subjects and subjects that suffer from other autoimmune diseases, liver diseases, neurological diseases, heart diseases, and infections also provide ChIP-Seq reads from blood samples. Disease load is calculated and death of specific cells related to the disease are calculated. Markers for various subtypes of the disease (such as are described hereinabove) are generated and multigene signatures are generated that allow for subtyping. Diseases characterized by immune cell death and in particular T cell death are examined as well. A T cell load score is calculated and markers for various T cell subtypes are generated along with a multigene signature for T cell subtyping.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of determining disease load in a subject suffering from a disease associated with cell death of a specific tissue or cell type, the method comprising:
a. receiving chromatin immunoprecipitation-sequencing (ChIP-Seq) reads from a plurality of genomic locations from cell free DNA (cfDNA) from blood samples from
i. a first population of control subjects;
ii. a second population of subjects suffering from said disease; and
iii. said subject; and
b. assigning a disease load score to said subject based on the similarity of said subject's reads to reads from said second population and dissimilarity to reads from said first population, wherein said score is proportional to said disease load in said subject;
thereby determining disease load in a subject.

2. (canceled)

3. (canceled)

4. (canceled)

5. The method of claim 1, wherein at least one of:
a. said similarity is determined by a linear regression analysis;
b. said ChIP-Seq was performed with an antibody to a DNA associated protein that marks active transcription selected from: histone H3 lysine 4 trimethylation (H3K4me3), histone H3 lysine 27 acetylation (H3K27Ac), histone H3 lysine 36 trimethylation (H3K36me3), histone H3 lysine 4 monomethylation (H3K4me) and histone H3 lysine 4 dimethylation (H3K4me2);
c. said similarity is determined by a trained machine learning algorithm, wherein said machine learning algorithm is trained on ChIP-Seq reads from cfDNA from blood samples from said first population and said second population and labels identifying said ChIP-Seq reads as being from a subject of the first population or a subject of the second population;
d. said control subjects are healthy subjects; and
e. said second population is a subset of said second population, wherein said subset comprises the top 10% of said second population with the most differentially expressed genes, based on ChIP-Seq reads, as compared to said first population.

6. (canceled)

7. (canceled)

8. (canceled)

9. The method of claim 1, wherein said disease is a specific type of cancer and said disease load score is a cancer load score or is a specific liver disease and said disease load score is a liver disease load score.

10. The method of claim 9, wherein at least one of:
a. said disease is a specific type and said control subjects are subjects that suffer from a cancer of a different type than said specific type of cancer;
b. said disease is lung cancer;
c. said disease is small cell lung cancer (SCLC) and wherein said score is specific to SCLC and not other cancers;
d. said disease is SCLC and a disease score beyond a predetermined threshold indicates said subject suffers from SCLC;
e. said disease is a specific liver disease and said control subject are subjects that suffer from a liver disease other than said specific liver disease;
f. said disease is autoimmune hepatitis (AIH), and wherein said disease load score is specific to AIH and not other liver diseases; and
g. said disease is autoimmune hepatitis (AIH) and a disease score beyond a predetermined threshold indicates said subject suffers from AIH.

11. (canceled)

12. (canceled)

13. (canceled)

14. (canceled)

15. (canceled)

16. (canceled)

17. (canceled)

18. The method of claim 1, wherein said receiving ChIP-Seq reads comprises:
a. receiving a blood sample from said subject, a subject of said first population, a subject from said second population or any combination thereof;
b. contacting said sample with at least one reagent that binds to a DNA-associated protein indicative of active transcription;
c. isolating said reagent and any thereto bound proteins and cfDNA; and
d. sequencing said cfDNA.

19. A method of determining a ChIP-Seq marker that distinguishes cfDNA from a first disease from cfDNA from a second disease, the method comprising:
a. determining disease load in a plurality of subjects suffering from said first disease by a method of claim 1 wherein said control subjects are healthy subjects or subjects suffering from a different disease;
b. selecting a subset of said plurality of subjects with a disease load above a predetermined threshold; and
c. comparing ChIP-Seq reads from cfDNA from blood from said subset with ChIP-Seq reads from cfDNA from blood of a third population of subjects suffering from said second disease and selecting at least one genomic region with a differential signal between said subset and said third population;
thereby determining a ChIP-Seq marker.

20. The method of claim 19, wherein at least one of:
a. said comparing is comparing ChIP-Seq reads from genomic regions with a differential signal between said first population and said second population;
b. said genomic regions are from within a gene body or regulatory element of a gene;
c. said genomic regions are from within a promoter; and
d. said genomic regions are from within a gene body and wherein said gene is a transcription factor or transcriptional coregulator.

21. The method of claim 19, wherein said method is a method of determining markers for a cancer subtype, wherein said first disease and second disease are cancer of the same type, the same tissue or cell type and said first disease is a first subtype of said cancer and said second disease is a second subtype of said cancer, optionally wherein said cancer is SCLC and said method is a method of determining a marker for a SCLC subtype.

22. (canceled)

23. (canceled)

24. (canceled)

25. (canceled)

26. A method of classifying a subject as suffering from a first disease, the method comprising:
a. determining a ChIP-Seq marker for said first disease by a method of claim 19;
b. receiving ChIP-Seq reads from cfDNA from a blood sample from said subject; and
c. identifying reads of said determined ChIP-Seq marker in said received ChIP-Seq reads, wherein reads above a predetermining threshold indicate said subject suffers from said first disease;
thereby classifying a subject as suffering from a first disease.

27. The method of claim 26, further comprising administering to said subject a therapeutic agent that treats said first disease.

28. A method of assigning a subject suffering from SCLC to a SCLC subtype, the method comprising:
a. receiving ChIP-Seq reads from cfDNA from a blood sample from said subject; and
b. identifying reads from at least one informative genomic locus as being above or below a predetermined threshold, wherein said reads are from a genomic locus provided in any one of Tables 1-3 wherein said SCLC subtype is selected from: achaete-scute homolog 1 (ASCL1) subtype, neurogenic differentiation 1 (NEUROD1) subtype, POU domain class 2 transcription factor 3 (POU2F3) subtype, yes-associated protein 1 (YAP1) subtype and protein atonal homolog 1 (ATOH1) subtype;
thereby assigning a subject suffering from SCLC to a SCLC subtype.

29. The method of claim 28, wherein at least one of:
a. said subtype is further selected from high neuroendocrine phenotype SCLC and non- or low-neuroendocrine phenotype SCLC;
b. said subtype is high neuroendocrine phenotype SCLC which is ASCL1 subtype, or a NEUROD1 subtype, or is non- or low-neuroendocrine phenotype SCLC which is a POU2F3 subtype, YAP1 subtype or an ATOH1 subject; and
c. said subtype is selected from: ASCL1, NEUROD1, POU2F3 and ATOH1 subtypes.

30. (canceled)

31. (canceled)

32. (canceled)

33. The method of claim 28, wherein
a. said reads are from a genomic locus provided in Table 1 and reads above a predetermined threshold indicate said SCLC is of the ASCL1 subtype;
b. said reads are from a genomic locus provided in Table 2 and reads above a predetermined threshold indicate said SCLC is of the NEUROD1 subtype; or
c. said reads are from a genomic locus provided in Table 3 and reads above a predetermined threshold indicate said SCLC is of the POU2F3 subtype.

34. (canceled)

35. (canceled)

36. The method of claim 28, wherein reads are from at least one genomic locus provided in each of Tables 1-3 and wherein reads from all genomic loci are below a predetermined threshold said SCLC is of the ATOH1 or YAP1 subtype.

37. (canceled)

38. (canceled)

39. The method of claim 28, further comprising administering to said subject a therapeutic agent specific to said SCLC subtype.

40. The method of claim 28, wherein said determined cancer subtype is high-neuroendocrine subtype and said therapeutic agent comprises chemotherapy or said determined cancer subtype is non- or low-neuroendocrine subtype and said therapeutic treatment comprises immunotherapy.

41. (canceled)

42. The method of claim 40, wherein said immunotherapy is immune checkpoint blockade, optionally wherein said immune checkpoint is PD-1/PD-L1.

43. A method of diagnosing or prognosing AIH in a subject, the method comprising:
a. receiving ChIP-Seq reads from cfDNA from a blood sample from said subject; and
b. identifying reads from an informative genomic locus as being above a predetermined threshold, wherein said reads are from a genomic locus within a gene body or promoter of a gene selected from: BCL2L14, CXCL10, CXCL11, CXCL9, GBP1, GBP5, HAPLN3, HLA-DOB, IL32, KB-1615E4.2, MARVELD3, OAS2, TRIM31, UBD, UPP2;
thereby diagnosing or prognosing AIH in a subject.

44. (canceled)

45. The method of claim 43, further comprising administering an anti-AIH therapeutic agent to a subject diagnosed with AIH or wherein said method is a method of monitoring AIH in a subject being administered an anti-AIH therapeutic agent and further comprises continuing to administer said anti-AIH therapeutic agent to a subject determined to have residual AIH.

46. (canceled)

47. (canceled)

48. The method of claim 45, wherein said anti-AIH therapeutic agent is an immunosuppressant, optionally wherein said immunosuppressant is a steroid.
